(19) United States
(12) Patent Application Publication
(10) Pub. No.: US 2013/0014293 A1
(43) Pub. Date: Jan. 10, 2013

(54) Xylanase Variants and Polynucleotides Encoding Same

(75) Inventors: Janine Lin, Davis, CA (US); Jung Ye, Sacramento, CA (US); Aubrey Jones, Davis, CA (US); Suzanne Otani, Elk Grove, CA (US); Tia Heu, Elk Grove, CA (US); Pierre Cassland, Vellinge (SE); Esben Friis, Herlev (DK)

(73) Assignees: NOVOZYMES A/S, Bagsvaerd (DK); NOVOZYMES, INC., Davis, CA (US)

(21) Appl. No.: 13/519,881

(22) PCT Filed: Mar. 2, 2011
(86) PCT No.: PCT/US11/26876

§ 371 Date: Sep. 24, 2012

Related U.S. Application Data
(60) Provisional application No. 61/310,136, filed on Mar. 3, 2010.

(51) Int. Cl. C12N 9/24; C12N 5/10; C12N 1/21; D21C 9/00; C12N 1/19; C12P 19/14; A01H 5/00; C12N 15/56; C12N 1/15

(52) U.S. Cl. 800/298; 435/200; 536/23.2; 435/419; 435/252.3; 435/252.31; 435/252.35; 435/252.33; 435/252.34; 435/325; 435/348; 435/254.11; 435/254.2; 435/254.22; 435/254.21; 435/254.23; 435/254.3; 435/254.7; 435/254.8; 435/254.4; 435/254.5; 435/254.6; 435/412; 435/414; 435/417; 435/415; 435/99; 162/72

(57) ABSTRACT

The present invention relates to variants of a parent xylanase. The present invention also relates to polynucleotides encoding the variants; nucleic acid constructs, vectors, and host cells comprising the polynucleotides; and methods of using the variants.

### REFERENCE TO A SEQUENCE LISTING

This application contains a Sequence Listing in computer readable form, which is incorporated herein by reference.

### BACKGROUND OF THE INVENTION

1. Field of the Invention

The present invention relates to variants of a xylanase, polynucleotides encoding the variants, methods of producing the variants, and methods of using the variants.

2. Description of the Related Art

Xylan, a major component of plant hemicellulose, is a polymer of D-xylose linked by beta-1,4-xylosidic bonds. Xylan can be degraded to xylose and xylo-oligomers by acid or enzymatic hydrolysis. Enzymatic hydrolysis of xylan produces free sugars without the by-products formed with acid (e.g., furans).

Xylanases can be used in various applications such as enzymatic breakdown of agricultural wastes for production of alcoholic fuels, enzymatic treatment of animal feeds to release free sugars, enzymatic treatment for dissolving pulp in the preparation of cellulose, and enzymatic treatment in biobleaching of pulp. In particular, xylanase is useful in the paper and pulp industry to enhance the brightness of bleached pulp, improve the quality of pulp, decrease the amount of chlorine used in the chemical pulp bleaching steps, and to increase the freeness of pulp in recycled paper processes.

Dumon et al., 2008, Journal of Biological Chemistry 283: 22557-22564, describe the engineering of hyperthermostability into a GH11 xylanase. Wang and Tao, 2008, Biotechnology Letters 30: 937-944, disclose the enhancement of the activity and alkaline pH stability of Thermobifida fusca xylanase A by directed evolution.

U.S. Pat. No. 5,759,840 discloses modification of Family 11 xylanases to improve thermophilicity, alkalophilicity and thermostability. U.S. Pat. No. 7,060,482 discloses modified xylanases comprising either a basic amino acid at position 162 corresponding to the Trichoderma reesei xylanase (TrX) amino acid sequence, or its equivalent position in other xylanase molecules, at least one disulfide bridge, or a combination thereof. U.S. Pat. No. 7,314,743 discloses a modified xylanase having at least one substituted amino acid residue at a position corresponding to the Trichoderma reesei xylanase II amino acid sequence. WO 2007/115391 discloses a modified Family 11 xylanase enzyme comprising cysteine residues at positions 99 and 118 corresponding to the Trichoderma reesei xylanase II amino acid sequence to form an intramolecular disulfide bond.

The present invention provides variants of a xylanase with improved properties compared to its parent enzyme.

### SUMMARY OF THE INVENTION

The present invention relates to isolated variants of a parent xylanase, comprising a substitution at one or more (several) positions corresponding to positions 2, 17, 21, 28, 38, 41, 55, 56, 57, 60, 62, 74, 81, 104, 111, 121, 151, 159, 161, 183, 186, 188, and 192 of the mature polypeptide of SEQ ID NO: 2 or SEQ ID NO: 4, wherein the variants have xylanase activity.

The present invention also relates to isolated polynucleotides encoding the variants; nucleic acid constructs, vectors, and host cells comprising the polynucleotides; and methods of producing the variants.

The present invention further relates to methods of degrading a xylan-containing material comprising treating the material with such a variant.

The present invention also relates to methods for treating a pulp, comprising contacting the pulp with such a variant.

The present invention further relates to methods of degrading a xylan-containing material comprising treating the material with such a variant.

The present invention further relates to methods or producing xylose, comprising contacting a xylan-containing material with such a variant.

### BRIEF DESCRIPTION OF THE FIGURES

FIG. 1 shows a restriction map of plasmid pTH025.

FIG. 2 shows a restriction map of plasmid pTH153.

FIG. 3 shows the DNA sequence and deduced amino acid sequence of a synthetic polynucleotide fragment comprising a Bacillus clausii serine protease ribosome binding site (RBS) and B. clausii serine protease signal sequence (underlined) fused to a 582 bp codon-optimized gene encoding T. fusca GH11 xylanase minus the cellulose binding domain.

FIGS. 4A and 4B show spectrophotometric and kappa number measurements of T. fusca xylanase variant 136 compared to wild-type T. fusca GH11 xylanase at 70° C. and pH 9.5.

FIGS. 5A and 5B show spectrophotometric and kappa number measurements of T. fusca xylanase variant 370 compared to wild-type T. fusca GH11 xylanase at 70° C. and pH 9.5

FIGS. 6A and 6B show spectrophotometric and kappa number measurements of T. fusca xylanase variant 566 compared to wild-type T. fusca GH11 xylanase at 80° C. and pH 9.5.

FIGS. 7A and 7B show spectrophotometric and kappa number measurements of T. fusca xylanase variant 564 compared to wild-type T. fusca GH11 xylanase at 80° C. and pH 9.5.

### DEFINITIONS

Xylanase activity: The term “xylanase” is defined herein as a 1,4-beta-D-xylan-xylanohydrolase (E.C. 3.2.1.8), which catalyzes the endohydrolysis of 1,4-beta-D-xylosidic linkages in xylans. For purposes of the present invention, xylanase activity is determined with 0.1% AZCL-xylan oat (Megazyme Wicklow, Ireland) as substrate in 0.01% TWEEN® 20-125 mM sodium borate pH 8.8 at 50° C. at 595 nm.

Variant: The term “variant” means a polypeptide having xylanase activity comprising an alteration, i.e., a substitution, insertion, and/or deletion of one or more (e.g., several) amino acid residues at one or more positions. A substitution means a replacement of the amino acid occupying a position with a different amino acid; a deletion means removal of the amino acid occupying a position; and an insertion means adding 1-3 amino acids adjacent to the amino acid occupying a position.

Mutant: The term “mutant” means a polynucleotide encoding a variant.

Wild-Type Enzyme: The term “wild-type” xylanase means a xylanase expressed by a naturally occurring microorganism, such as a bacterium, yeast, or filamentous fungus found in nature.

Parent or parent xylanase: The term “parent” or “parent xylanase” means a xylanase to which an alteration is made to produce the enzyme variants of the present invention. The parent may be a naturally occurring (wild-type) polypeptide or a variant thereof.

Isolated or purified: The terms “isolated” and “purified” mean a polypeptide or polynucleotide that is removed from at least one component with which it is naturally associated. For example, a variant may be at least 1% pure, e.g., at least 5% pure, at least 10% pure, at least 20% pure, at least 40% pure, at least 60% pure, at least 80% pure, at least 90% pure, and at least 95% pure, as determined by SDS-PAGE and a polynucleotide may be at least 1% pure, e.g., at least 5% pure, at least 10% pure, at least 20% pure, at least 40% pure, at least 60% pure, at least 80% pure, at least 90% pure, and at least 95% pure, as determined by agarose electrophoresis.

Mature polypeptide: The term “mature polypeptide” means a polypeptide in its final form following translation and any post-translational modifications, such as N-terminal processing, C-terminal truncation, glycosylation, phosphorylation, etc. In one aspect, the mature polypeptide is amino acids 1 to 194 of SEQ ID NO: 2 based on the SignalP program (Nielsen et al., 1997, Protein Engineering 10:1-6) that predicts amino acids −1 to −27 of SEQ ID NO: 2 are a signal peptide. In another aspect, the mature polypeptide is amino acids 1 to 296 of SEQ ID NO: 4 based on the SignalP program (Nielsen et al., 1997, supra) that predicts amino acids −1 to −42 of SEQ ID NO: 4 are a signal peptide.

Mature polypeptide coding sequence: The term “mature polypeptide coding sequence” means a polynucleotide that encodes a mature polypeptide having xylanase activity. In one aspect, the mature polypeptide coding sequence is nucleotides 82 to 663 of SEQ ID NO: 1 based on the SignalP program (Nielsen et al., 1997, supra) that predicts nucleotides 1 to 81 of SEQ ID NO: 1 encode a signal peptide. In another aspect, the mature polypeptide coding sequence is nucleotides 127 to 1014 of SEQ ID NO: 3 based on the SignalP program (Nielsen et al., 1997, supra) that predicts nucleotides 1 to 126 of SEQ ID NO: 3 encode a signal peptide

Sequence Identity: The relatedness between two amino acid sequences or between two deoxyribonucleotide sequences is described by the parameter “sequence identity”.

For purposes of the present invention, the degree of sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 3.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical deoxyribonucleotides×100)/(Length of Alignment−Total Number of Gaps in Alignment)

Polypeptide fragment: The term “polypeptide fragment” means a polypeptide having one or more (several) amino acids deleted from the amino and/or carboxyl terminus of a mature polypeptide; wherein the fragment has xylanase activity. In one aspect, a fragment contains at least 160 amino acid residues, e.g., at least 170 amino acid residues or at least 180 amino acid residues. In another aspect, a fragment contains at least 255 amino acid residues, e.g., at least 270 amino acid residues or at least 285 amino acid residues.

Subsequence: The term “subsequence” means a polynucleotide sequence having one or more (several) nucleotides deleted from the 5′ and/or 3′ end of a mature polypeptide coding sequence; wherein the subsequence encodes a polypeptide fragment having xylanase activity. In one aspect, a subsequence contains at least 480 nucleotides, e.g., at least 510 nucleotides or at least 540 nucleotides. In another aspect, a subsequence contains at least 765 nucleotides, e.g., at least 810 nucleotides or at least 855 nucleotides.

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Coding sequence: The term “coding sequence” means a polynucleotide, which directly specifies the amino acid sequence of its polypeptide product. The boundaries of the coding sequence are generally determined by an open reading frame, which usually begins with the ATG start codon or alternative start codons such as GTG and TTG and ends with a stop codon such as TAA, TAG, and TGA. The coding sequence may be a DNA, cDNA, synthetic, or recombinant polynucleotide.

cDNA: The term “cDNA” is defined herein as a DNA molecule that can be prepared by reverse transcription from a mature, spliced, mRNA molecule obtained from a eukaryotic cell. cDNA lacks intron sequences that may be present in the corresponding genomic DNA. The initial, primary RNA transcript is a precursor to mRNA that is processed through a series of steps including splicing before appearing as mature spliced mRNA.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present invention.

Control sequences: The term “control sequences” means nucleic acid sequences necessary for the expression of a polynucleotide encoding a variant of the present invention. Each control sequence may be native (i.e., from the same gene) or foreign (i.e., from a different gene) to the polynucleotide encoding the variant or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a variant.

Operably linked: The term “operably linked” means a configuration in which a control sequence is placed at an appropriate position relative to the coding sequence of a polynucleotide sequence such that the control sequence directs the expression of the coding sequence of a polypeptide.

Expression: The term “expression” includes any step involved in the production of the variant including, but not limited to, transcription, post-transcriptional modification, translation, post-translational modification, and secretion.

Expression vector: The term “expression vector” means a linear or circular DNA molecule that comprises a polynucleotide encoding a variant and is operably linked to additional nucleotides that provide for its expression.

Host cell: The term “host cell” means any cell type that is susceptible to transformation, transfection, transduction, and the like with a nucleic acid construct or expression vector comprising a polynucleotide of the present invention. The term “host cell” encompasses any progeny of a parent cell that is not identical to the parent cell due to mutations that occur during replication.

Improved property: The term “improved property” means a characteristic associated with a variant that is improved compared to the parent. Such improved properties include, but are not limited to, thermal activity, thermostability, pH activity, pH stability, substrate/cofactor specificity, improved surface properties, product specificity, increased stability or solubility in the presence of pretreated biomass, improved stability under storage conditions, and chemical stability.

Improved thermostability: The term “improved thermostability” means a variant displaying retention of xylanase activity after a period of incubation at a temperature relative to the parent, either in a buffer or under conditions such as those which exist during product storage/transport or conditions similar to those that exist during industrial use of the variant. The temperature can be any suitable temperature where a difference in thermostability between the variant and parent can be observed, e.g., 40° C., 45° C., 50° C., 55° C., 60° C., 65° C., 70° C., 75° C., 80° C., 85° C., 90° C., 95° C., or any other suitable temperature. The pH for determining improved thermostability can be any suitable pH, e.g., 3, 3.5, 4, 4.5 5, 5.5, 6, 6.5, 7, 7.5, 8, 8.5, 9, 9.5, 10, 10.5, 11, or any other suitable pH. A variant having improved thermostability may or may not display an altered thermal activity profile relative to the parent. For example, a variant may have an improved ability to refold following incubation at an elevated temperature relative to the parent.

In an aspect, the thermostability of the variant having xylanase activity is at least 1.05-fold, e.g., at least 1.1-fold, at least 1.2-fold, at least 1.3-fold, at least 1.4-fold, at least 1.5-fold, at least 2-fold, at least 2.5-fold, at least 3-fold, at least 3.5-fold, at least 4-fold, at least 4.5-fold, and at least 5-fold more thermostable than the parent when residual activity is compared using an appropriate assay such as the assay described in Example 7.

Improved thermal activity: The term “improved thermal activity” means a variant displaying an altered temperature-dependent activity profile in a specific temperature range relative to the temperature-dependent activity profile of the parent. The temperature range can be any suitable temperature range where a difference in thermal activity between the variant and parent can be observed. The pH for determining improved thermal activity can be any suitable pH, e.g., 3, 3.5, 4, 4.5 5, 5.5, 6, 6.5, 7, 7.5, 8, 8.5, 9, 9.5, 10, 10.5, 11, or any other suitable pH. The thermal activity value provides a measure of the variant's efficiency in enhancing catalysis of a hydrolysis reaction over a range of temperatures. A variant is stable and retains its activity in a specific temperature range, but becomes less stable and thus less active with increasing temperature. Furthermore, the initial rate of a reaction catalyzed by a variant can be accelerated by an increase in temperature that is measured by determining thermal activity of the variant. A more thermoactive variant will lead to an increase in enhancing the rate of hydrolysis of a substrate by an enzyme composition thereby decreasing the time required and/or decreasing the enzyme concentration required for activity. Alternatively, a variant with reduced thermal activity will enhance an enzymatic reaction at a temperature lower than the temperature optimum of the parent defined by the temperature-dependent activity profile of the parent.

In an aspect, the thermal activity of the variant is at least 1.05-fold, e.g., at least 1.1-fold, at least 1.2-fold, at least 1.3-fold, at least 1.4-fold, at least 1.5-fold, at least 2-fold, at least 3-fold, at least 4-fold, at least 5-fold, at least 10-fold, at least 20-fold, at least 30-fold, at least 40-fold, at least 50-fold, at least 60-fold, at least 70-fold, at least 80-fold, at least 90-fold, at least 100-fold, at least 125-fold, at least 150-fold, at least 175-fold, and at least 200-fold more thermally active than the parent when residual activity is compared using an appropriate assay such as the assay described in Example 8.

Improved bleach boosting performance: The term “improved bleach boosting performance” means a variant yielding higher Kappa number reduction and release of 280 nm absorbing material from a pulp than the parent. The temperature can be any suitable temperature where a difference in bleach boosting performance between the variant and parent can be observed, e.g., 40° C., 45° C., 50° C., 55° C., 60° C., 65° C., 70° C., 75° C., 80° C., 85° C., 90° C., 95° C., or any other suitable temperature. The pH for determining improved bleach boosting performance can be any suitable pH, e.g., 3, 3.5, 4, 4.5 5, 5.5, 6, 6.5, 7, 7.5, 8, 8.5, 9, 9.5, 10, 10.5, 11, or any other suitable pH.

In one aspect, treatment of a pulp with a variant of the present invention increases the bleach boosting performance at least 0.5%, e.g., at least 1%, at least 1.5%, at least 2%, at least 2.5%, at least 3%, at least 3.5%, at least 4%, at least 4.5%, at least 5%, at least 7.5%, and at least 10% compared to treatment with the parent based on the Kappa number reduction of the pulp, using an appropriate assay such as the assay described in Example 13.

In another aspect, treatment of a pulp with a variant of the present invention increases the bleach boosting performance 1.05-fold, e.g., at least 1.1-fold, at least 1.2-fold, at least 1.3-fold, at least 1.4-fold, at least 1.5-fold, at least 2-fold, at least 3-fold, at least 4-fold, at least 5-fold, and at least 10-fold compared to treatment with the parent based on the release of 280 nm absorbing material from the pulp (see Example 13).

Xylan-containing material: The term “xylan-containing material” is defined herein as any material comprising a plant cell wall polysaccharide containing a backbone of beta-(1-4)-linked xylose residues. Xylans of terrestrial plants are heteropolymers possessing a beta-(1-4)-D-xylopyranose backbone, which is branched by short carbohydrate chains. They comprise D-glucuronic acid or its 4-O-methyl ether, L-arabinose, and/or various oligosaccharides, composed of D-xylose, L-arabinose, D- or L-galactose, and D-glucose. Xylan-type polysaccharides can be divided into homoxylans and heteroxylans, which include glucuronoxylans, (arabino)glucuronoxylans, (glucurono)arabinoxylans, arabinoxylans, and complex heteroxylans. See, for example, Ebringerova et al., 2005, Adv. Polym. Sci. 186: 1-67.

In the methods of the present invention, any material containing xylan may be used. In a preferred aspect, the xylan-containing material is 8myl8chyma8ydes.

### DETAILED DESCRIPTION OF THE INVENTION

The present invention relates to isolated variants of a parent xylanase, comprising a substitution at one or more (several) positions corresponding to positions 2, 17, 21, 28, 38, 41, 55, 56, 57, 60, 62, 74, 81, 104, 111, 121, 151, 159, 161, 183, 186, 188, and 192 of the mature polypeptide of SEQ ID NO: 2 or SEQ ID NO: 4, wherein the variants have xylanase activity.

### Conventions for Designation of Variants

For purposes of the present invention, the mature polypeptide disclosed in SEQ ID NO: 2 or SEQ ID NO: 4 is used to determine the corresponding amino acid residue in another xylanase. The amino acid sequence of another xylanase is aligned with the mature polypeptide disclosed in SEQ ID NO: 2 or SEQ ID NO: 4, and based on the alignment, the amino acid position number corresponding to any amino acid residue in the mature polypeptide disclosed in SEQ ID NO: 2 or SEQ ID NO: 4 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 3.0.0 or later.

Identification of the corresponding amino acid residue in another xylanase can be confirmed by an alignment of multiple polypeptide sequences using “ClustalW” (Larkin et al., 2007, Bioinformatics 23: 2947-2948).

When the other enzyme has diverged from the mature polypeptide of SEQ ID NO: 2 or SEQ ID NO: 4 such that traditional sequence-based comparison fails to detect their relationship (Lindahl and Elofsson, 2000, J. Mol. Biol. 295: 613-615), other pairwise sequence comparison algorithms can be used. Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Atschul et al., 1997, Nucleic Acids Res. 25: 3389-3402). Even greater sensitivity can be achieved if the family or superfamily for the polypeptide has one or more representatives in the protein structure databases. Programs such as GenTHREADER (Jones, 1999, J. Mol. Biol. 287: 797-815; McGuffin and Jones, 2003, Bioinformatics 19: 874-881) utilize information from a variety of sources (PSI-BLAST, secondary structure prediction, structural alignment profiles, and 9myl9chym potentials) as input to a neural network that predicts the structural fold for a query sequence. Similarly, the method of Gough et al., 2000, J. Mol. Biol. 313: 903-919, can be used to align a sequence of unknown structure with the superfamily models present in the SCOP database. These alignments can in turn be used to generate homology models for the polypeptide, and such models can be assessed for accuracy using a variety of tools developed for that purpose.

For proteins of known structure, several tools and resources are available for retrieving and generating structural alignments. For example the SCOP superfamilies of proteins have been structurally aligned, and those alignments are accessible and downloadable. Two or more protein structures can be aligned using a variety of algorithms such as the distance alignment matrix (Holm and Sander, 1998, Proteins 33: 88-96) or combinatorial extension (Shindyalov and Bourne, 1998, Protein Engineering 11: 739-747), and implementations of these algorithms can additionally be utilized to query structure databases with a structure of interest in order to discover possible structural homologs (e.g., Holm and Park, 2000, Bioinformatics 16: 566-567).

In describing the xylanase variants of the present invention, the nomenclature described below is adapted for ease of reference. The accepted IUPAC single letter or three letter amino acid abbreviation is employed.

Substitutions.

For an amino acid substitution, the following nomenclature is used: Original amino acid, position, substituted amino acid. Accordingly, the substitution of threonine with alanine at position 226 is designated as “Thr226Ala” or “T226A”. Multiple mutations are separated by addition marks (“+”), e.g., “Gly205Arg+Ser411Phe” or “G205R+S411F”, representing substitutions at positions 205 and 411 of glycine (G) with arginine I, and serine (S) with phenylalanine (F), respectively.

Deletions.

For an amino acid deletion, the following nomenclature is used: Original amino acid, position*. Accordingly, the deletion of glycine at position 195 is designated as “Gly195*” or “G195*”. Multiple deletions are separated by addition marks (“+”), e.g., “Gly195*+Ser411*” or “G195*+S411*”.

Insertions.

For an amino acid insertion, the following nomenclature is used: Original amino acid, position, original amino acid, new inserted amino acid. Accordingly the insertion of lysine after glycine at position 195 is designated “Gly195GlyLys” or “G195GK”. An insertion of multiple amino acids is designated [Original amino acid, position, original amino acid, inserted amino acid #1, new inserted amino acid #2; etc.]. For example, the insertion of lysine and alanine after glycine at position 195 is indicated as “Gly195GlyLysAla” or “G195GKA”.

In such cases the inserted amino acid residue(s) are numbered by the addition of lower case letters to the position number of the amino acid residue preceding the inserted amino acid residue(s). In the above example, the sequence would thus be:

| Parent: | Variant: |
|---|---|
| 195 | 195 195a 195b |
| G | G - K - A |

Multiple Alterations.

Variants comprising multiple alterations are separated by addition marks (“+”), e.g., “Arg170Tyr+Gly195Glu” or “R170Y+G195E” representing a substitution of tyrosine and glutamic acid for arginine and glycine at positions 170 and 195, respectively.

Different Alterations.

Where different alterations can be introduced at a position, the different alterations are separated by a comma, e.g., “Arg170Tyr,Glu” represents a substitution of arginine with tyrosine or glutamic acid at position 170. Thus, “Tyr167Gly,Ala+Arg170Gly,Ala” designates the following variants: “Tyr167Gly+Arg170Gly”, “Tyr167Gly+Arg170Ala”, “Tyr167Ala+Arg170Gly”, and “Tyr167Ala+Arg170Ala”.

### Parent Xylanases

The parent xylanase may be (a) a polypeptide having at least 60% sequence identity to the mature polypeptide of SEQ ID NO: 2 or SEQ ID NO: 4; (b) a polypeptide encoded by a polynucleotide that hybridizes under at least low stringency conditions with the mature polypeptide coding sequence of SEQ ID NO: 1 or SEQ ID NO: 3, or their full-length complementary strands; or (c) a polypeptide encoded by a polynucleotide having at least 60% sequence identity to the mature polypeptide coding sequence of SEQ ID NO: 1 or SEQ ID NO: 3.

In a first aspect, the parent has a sequence identity to the mature polypeptide of SEQ ID NO: 2 or SEQ ID NO: 4 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have xylanase activity. In one aspect, the amino acid sequence of the parent differs by no more than ten amino acids, e.g., by nine amino acids, by eight amino acids, by seven amino acids, by six amino acids, by five amino acids, by four amino acids, by three amino acids, by two amino acids, and by one amino acid from the mature polypeptide of SEQ ID NO: 2 or SEQ ID NO: 4.

The parent preferably comprises or consists of the amino acid sequence of SEQ ID NO: 2 or SEQ ID NO: 4. In another aspect, the parent comprises or consists of the mature polypeptide of SEQ ID NO: 2 or SEQ ID NO: 4. In another aspect, the parent comprises or consists of amino acids 1 to 194 of SEQ ID NO: 2 or amino acids 1 to 296 of SEQ ID NO: 4.

In an embodiment, the parent is a fragment of the mature polypeptide of SEQ ID NO: 2 containing at least 160 amino acid residues, e.g., at least 165, at least 170, at least 175, at least 180, at least 185, or at least 190 amino acids.

In another embodiment, the parent is a fragment of the mature polypeptide of SEQ ID NO: 4 containing at least 250 amino acid residues, e.g., at least 255, at least 260, at least 265, at least 270, at least 275, at least 280, at least 285, at least 290, or at least 295 amino acids.

In another embodiment, the parent is an allelic variant of the mature polypeptide of SEQ ID NO: 2 or SEQ ID NO: 4.

In a second aspect, the parent is encoded by a polynucleotide that hybridizes under very low stringency conditions, low stringency conditions, medium stringency conditions, medium-high stringency conditions, high stringency conditions, or very high stringency conditions with the mature polypeptide coding sequence of SEQ ID NO: 1 or SEQ ID NO: 3, or their full-length complementary strands (full-length complement) (J. Sambrook, E.F. Fritsch, and T. Maniatis, 1989, Molecular Cloning, A Laboratory Manual, 2d edition, Cold Spring Harbor, N.Y.).

The polynucleotide of SEQ ID NO: 1 or SEQ ID NO: 3, or a subsequence thereof, as well as the polypeptide of SEQ ID NO: 2 or SEQ ID NO: 4, or a fragment thereof, may be used to design nucleic acid probes to identify and clone DNA encoding a parent from strains of different genera or species according to methods well known in the art. In particular, such probes can be used for hybridization with the genomic or cDNA of the genus or species of interest, following standard Southern blotting procedures, in order to identify and isolate the corresponding gene therein. Such probes can be considerably shorter than the entire sequence, but should be at least 14, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other organisms may be screened for DNA that hybridizes with the probes described above and encodes a parent. Genomic or other DNA from such other organisms may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that hybridizes with SEQ ID NO: 1 or SEQ ID NO: 3, or a subsequence thereof, the carrier material is used in a Southern blot.

For purposes of the present invention, hybridization indicates that the polynucleotide hybridizes to a labeled nucleotide probe corresponding to the polynucleotide shown in SEQ ID NO: 1 or SEQ ID NO: 3, the mature polypeptide coding sequence thereof, the full-length complementary strand thereof, or a subsequence thereof, under low to very high stringency conditions. Molecules to which the probe hybridizes can be detected using, for example, X-ray film or any other detection means known in the art.

In one aspect, the nucleic acid probe is the mature polypeptide coding sequence of SEQ ID NO: 1 or SEQ ID NO: 3. In another aspect, the nucleic acid probe is nucleotides 82 to 663 of SEQ ID NO: 1 or nucleotides 127 to 1014 of SEQ ID NO: 3. In another aspect, the nucleic acid probe is a polynucleotide that encodes the polypeptide of SEQ ID NO: 2 or SEQ ID NO: 4 or a fragment thereof. In another aspect, the nucleic acid probe is SEQ ID NO: 1 or SEQ ID NO: 3.

For long probes of at least 100 nucleotides in length, very low to very high stringency conditions are defined as prehybridization and hybridization at 42° C. in 5×SSPE, 0.3% SDS, 200 micrograms/ml sheared and denatured salmon sperm DNA, and either 25% formamide for very low and low stringencies, 35% formamide for medium and medium-high stringencies, or 50% formamide for high and very high stringencies, following standard Southern blotting procedures for 12 to 24 hours optimally. The carrier material is finally washed three times each for 15 minutes using 2×SSC, 0.2% SDS at 45° C. (very low stringency), 50° C. (low stringency), 55° C. (medium stringency), 60° C. (medium-high stringency), 65° C. (high stringency), or 70° C. (very high stringency).

For short probes that are about 15 nucleotides to about 70 nucleotides in length, stringency conditions are defined as prehybridization and hybridization at about 5° C. to about 10° C. below the calculated Tₘ using the calculation according to Bolton and McCarthy (1962, Proc. Natl. Acad. Sci. USA 48: 1390) in 0.9 M NaCl, 0.09 M Tris-HCl pH 7.6, 6 mM EDTA, 0.5% NP-40, 1×Denhardt's solution, 1 mM sodium pyrophosphate, 1 mM sodium monobasic phosphate, 0.1 mM ATP, and 0.2 mg of yeast RNA per ml following standard Southern blotting procedures for 12 to 24 hours optimally. The carrier material is finally washed once in 6×SCC plus 0.1% SDS for 15 minutes and twice each for 15 minutes using 6×SSC at 5° C. to 10° C. below the calculated Tₘ.

In a third aspect, the parent is encoded by a polynucleotide with a sequence identity to the mature polypeptide coding sequence of SEQ ID NO: 1 or SEQ ID NO: 3 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which encodes a polypeptide having xylanase activity. In one aspect, the mature polypeptide coding sequence is nucleotides 82 to 663 of SEQ ID NO: 1 or nucleotides 127 to 1014 of SEQ ID NO: 3. In an embodiment, the parent is encoded by a polynucleotide comprising or consisting of SEQ ID NO: 1 or SEQ ID NO: 3.

The parent may be obtained from microorganisms of any genus. For purposes of the present invention, the term “obtained from” as used herein in connection with a given source shall mean that the parent encoded by a polynucleotide is produced by the source or by a cell in which the polynucleotide from the source has been inserted. In one aspect, the parent is secreted extracellularly.

The parent may be a bacterial xylanase. For example, the parent may be a gram-positive bacterial polypeptide such as a Bacillus, Clostridium, Enterococcus, Geobacillus, Lactobacillus, Lactococcus, Oceanobacillus, Staphylococcus, Streptococcus, or Streptomyces xylanase, or a gram-negative bacterial polypeptide such as a Campylobacter, Dictyoglomus, E. coli, Flavobacterium, Fusobacterium, Helicobacter, Ilyobacter, Neisseria, Pseudomonas, Salmonella, Thermotoga, or Ureaplasma xylanase.

In one aspect, the parent is a Bacillus alkalophilus, Bacillus amyloliquefaciens, Bacillus brevis, Bacillus circulans, Bacillus clausii, Bacillus coagulans, Bacillus firmus, Bacillus halodurans, Bacillus lautus, Bacillus lentus, Bacillus licheniformis, Bacillus megaterium, Bacillus pumilus, Bacillus stearothermophilus, Bacillus subtilis, or Bacillus thuringiensis xylanase.

In another aspect, the parent is a Streptococcus equisimilis, Streptococcus pyogenes, Streptococcus uberis, or Streptococcus equi subsp. Zooepidemicus xylanase.

In another aspect, the parent is a Streptomyces achromogenes, Streptomyces avermitilis, Streptomyces coelicolor, Streptomyces griseus, or Streptomyces lividans xylanase.

In another aspect, the parent is a Dictyoglomus thermophilum or Thermotoga 14myl14chy xylanase.

The parent may be a fungal xylanase. For example, the parent may be a yeast xylanase such as a Candida, Kluyveromyces, Pichia, Saccharomyces, Schizosaccharomyces, or Yarrowia xylanase. For example, the parent may be a filamentous fungal xylanase such as an Acremonium, Agaricus, Alternaria, Aspergillus, Aureobasidium, Botryospaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thermomyces, Thielavia, Tolypocladium, Trichoderma, Trichophaea, Verticillium, Volvariella, or Xylaria xylanase.

In another aspect, the parent is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis xylanase.

In another aspect, the parent is an Acremonium cellulolyticus, Aspergillus aculeatus, Aspergillus awamori, Aspergillus foetidus, Aspergillus fumigatus, Aspergillus japonicus, Aspergillus nidulans, Aspergillus niger, Aspergillus oryzae, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Dictyoglomus thermophilum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinurn, Fusarium sarcochroum, Fusarium sporotrichioides, Fusarium sulphureum, Fusarium torulosum, Fusarium trichothecioides, Fusarium venenatum, Humicola grisea, Humicola insolens, Humicola 14myl14chyma, Irpex lacteus, Mucor miehei, Myceliophthora thermophila, Neurospora crassa, Penicillium funiculosum, Penicillium purpurogenum, Phanerochaete chrysosporium, Thermomyces lanuginosus, Thielavia achromatica, Thielavia albomyces, Thielavia albopilosa, Thielavia australeinsis, Thielavia fimeti, Thielavia microspora, Thielavia ovispora, Thielavia peruviana, Thielavia setosa, Thielavia spededonium, Thielavia subthermophila, Thielavia terrestris, Trichoderma harzianum, Trichoderma koningii, Trichoderma longibrachiatum, Trichoderma reesei, or Trichoderma viride xylanase.

In another aspect, the parent is a Thermobifida fusca xylanase, and preferably the Thermobifida fusca xylanase of SEQ ID NO: 2 or SEQ ID NO: 4 or the mature polypeptide thereof.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL).

The parent may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. The polynucleotide encoding a parent may then be derived by similarly screening a genomic or cDNA library of another microorganism or mixed DNA sample. Once a polynucleotide encoding a parent has been detected with a probe(s), the polynucleotide may be isolated or cloned by utilizing techniques that are known to those of ordinary skill in the art (see, e.g., Sambrook et al., 1989, supra).

The parent may be a hybrid polypeptide in which a portion of one polypeptide is fused at the N-terminus or the C-terminus of a portion of another polypeptide.

The parent also may be a fusion polypeptide or cleavable fusion polypeptide in which one polypeptide is fused at the N-terminus or the C-terminus of another polypeptide. A fusion polypeptide is produced by fusing a polynucleotide encoding one polypeptide to a polynucleotide encoding another polypeptide. Techniques for producing fusion polypeptides are known in the art, and include ligating the coding sequences encoding the polypeptides so that they are in frame and that expression of the fusion polypeptide is under control of the same promoter(s) and terminator. Fusion proteins may also be constructed using intein technology in which fusions are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

### Preparation of Variants

The present invention also relates to methods for obtaining a variant having xylanase activity, comprising: (a) introducing into a parent xylanase a substitution at one or more (several) positions corresponding to positions 2, 17, 21, 28, 38, 41, 55, 56, 57, 60, 62, 74, 81, 104, 111, 121, 151, 159, 161, 183, 186, 188, and 192 of the mature polypeptide of SEQ ID NO: 2 or SEQ ID NO: 4, wherein the variant has xylanase activity; and (b) recovering the variant.

The variants can be prepared using any mutagenesis procedure known in the art, such as site-directed mutagenesis, synthetic gene construction, semi-synthetic gene construction, random mutagenesis, shuffling, etc.

Site-directed mutagenesis is a technique in which one or more (several) mutations are created at one or more defined sites in a polynucleotide encoding the parent.

Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent and subsequent ligation of an oligonucleotide containing the mutation in the polynucleotide. Usually the restriction enzyme that digests the plasmid and the oligonucleotide is the same, permitting sticky ends of the plasmid and insert to ligate to one another. See, e.g., Scherer and Davis, 1979, Proc. Natl. Acad. Sci. USA 76: 4949-4955; and Barton et al., 1990, Nucleic Acids Res. 18: 7349-4966.

Site-directed mutagenesis can also be accomplished in vivo by methods known in the art. See, e.g., U.S. Patent Application Publication No. 2004/0171154; Storici et al., 2001, Nature Biotechnol. 19: 773-776; Kren et al., 1998, Nat. Med. 4: 285-290; and Calissano and Macino, 1996, Fungal Genet. Newslett. 43: 15-16.

Any site-directed mutagenesis procedure can be used in the present invention. There are many commercial kits available that can be used to prepare variants.

Synthetic gene construction entails in vitro synthesis of a designed polynucleotide molecule to encode a polypeptide of interest. Gene synthesis can be performed utilizing a number of techniques, such as the multiplex microchip-based technology described by Tian et al. (2004, Nature 432: 1050-1054) and similar technologies wherein oligonucleotides are synthesized and assembled upon photo-programmable microfluidic chips.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204) and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

Semi-synthetic gene construction is accomplished by combining aspects of synthetic gene construction, and/or site-directed mutagenesis, and/or random mutagenesis, and/or shuffling. Semi-synthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide subsequences may then be shuffled.

### Variants

The present invention also provides variants of a parent xylanase comprising a substitution at one or more (several) positions corresponding to positions 2, 17, 21, 28, 38, 41, 55, 56, 57, 60, 62, 74, 81, 104, 111, 121, 151, 159, 161, 183, 186, 188, and 192, wherein the variant has xylanase activity.

In an embodiment, the variant has sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of the parent xylanase.

In another embodiment, the variant has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, and at least 99%, but less than 100%, sequence identity with the mature polypeptide of SEQ ID NO: 2 or SEQ ID NO: 4.

In one aspect, the number of substitutions in the variants of the present invention is 1-23, e.g., 1-15, 1-10, and 1-5, such as 1, 2, 3, 4, 5, 6, 7, 8, 9, 10, 11, 12, 13, 14, 15, 16, 17, 18, 19, 20, 21, 22, or 23 substitutions.

In one aspect, a variant comprises a substitution at one or more (several) positions corresponding to any of positions 2, 17, 21, 28, 38, 41, 55, 56, 57, 60, 62, 74, 81, 104, 111, 121, 151, 159, 161, 183, 186, 188, and 192. In another aspect, a variant comprises a substitution at two positions corresponding to any of positions 2, 17, 21, 28, 38, 41, 55, 56, 57, 60, 62, 74, 81, 104, 111, 121, 151, 159, 161, 183, 186, 188, and 192. In another aspect, a variant comprises a substitution at three positions corresponding to any of positions 2, 17, 21, 28, 38, 41, 55, 56, 57, 60, 62, 74, 81, 104, 111, 121, 151, 159, 161, 183, 186, 188, and 192. In another aspect, a variant comprises a substitution at four positions corresponding to any of positions 2, 17, 21, 28, 38, 41, 55, 56, 57, 60, 62, 74, 81, 104, 111, 121, 151, 159, 161, 183, 186, 188, and 192. In another aspect, a variant comprises a substitution at five positions corresponding to any of positions 2, 17, 21, 28, 38, 41, 55, 56, 57, 60, 62, 74, 81, 104, 111, 121, 151, 159, 161, 183, 186, 188, and 192. In another aspect, a variant comprises a substitution at six positions corresponding to any of positions 2, 17, 21, 28, 38, 41, 55, 56, 57, 60, 62, 74, 81, 104, 111, 121, 151, 159, 161, 183, 186, 188, and 192. In another aspect, a variant comprises a substitution at seven positions corresponding to any of positions 2, 17, 21, 28, 38, 41, 55, 56, 57, 60, 62, 74, 81, 104, 111, 121, 151, 159, 161, 183, 186, 188, and 192. In another aspect, a variant comprises a substitution at eight positions corresponding to any of positions 2, 17, 21, 28, 38, 41, 55, 56, 57, 60, 62, 74, 81, 104, 111, 121, 151, 159, 161, 183, 186, 188, and 192. In another aspect, a variant comprises a substitution at nine positions corresponding to any of positions 2, 17, 21, 28, 38, 41, 55, 56, 57, 60, 62, 74, 81, 104, 111, 121, 151, 159, 161, 183, 186, 188, and 192. In another aspect, a variant comprises a substitution at ten positions corresponding to any of positions 2, 17, 21, 28, 38, 41, 55, 56, 57, 60, 62, 74, 81, 104, 111, 121, 151, 159, 161, 183, 186, 188, and 192. In another aspect, a variant comprises a substitution at eleven positions corresponding to any of positions 2, 17, 21, 28, 38, 41, 55, 56, 57, 60, 62, 74, 81, 104, 111, 121, 151, 159, 161, 183, 186, 188, and 192. In another aspect, a variant comprises a substitution at twelve positions corresponding to any of positions 2, 17, 21, 28, 38, 41, 55, 56, 57, 60, 62, 74, 81, 104, 111, 121, 151, 159, 161, 183, 186, 188, and 192. In another aspect, a variant comprises a substitution at thirteen positions corresponding to any of positions 2, 17, 21, 28, 38, 41, 55, 56, 57, 60, 62, 74, 81, 104, 111, 121, 151, 159, 161, 183, 186, 188, and 192. In another aspect, a variant comprises a substitution at fourteen positions corresponding to any of positions 2, 17, 21, 28, 38, 41, 55, 56, 57, 60, 62, 74, 81, 104, 111, 121, 151, 159, 161, 183, 186, 188, and 192. In another aspect, a variant comprises a substitution at fifteen positions corresponding to any of positions 2, 17, 21, 28, 38, 41, 55, 56, 57, 60, 62, 74, 81, 104, 111, 121, 151, 159, 161, 183, 186, 188, and 192. In another aspect, a variant comprises a substitution at sixteen positions corresponding to any of positions 2, 17, 21, 28, 38, 41, 55, 56, 57, 60, 62, 74, 81, 104, 111, 121, 151, 159, 161, 183, 186, 188, and 192. In another aspect, a variant comprises a substitution at seventeen positions corresponding to any of positions 2, 17, 21, 28, 38, 41, 55, 56, 57, 60, 62, 74, 81, 104, 111, 121, 151, 159, 161, 183, 186, 188, and 192. In another aspect, a variant comprises a substitution at eighteen positions corresponding to any of positions 2, 17, 21, 28, 38, 41, 55, 56, 57, 60, 62, 74, 81, 104, 111, 121, 151, 159, 161, 183, 186, 188, and 192. In another aspect, a variant comprises a substitution at nineteen positions corresponding to any of positions 2, 17, 21, 28, 38, 41, 55, 56, 57, 60, 62, 74, 81, 104, 111, 121, 151, 159, 161, 183, 186, 188, and 192.

In another aspect, a variant comprises a substitution at twenty positions corresponding to any of positions 2, 17, 21, 28, 38, 41, 55, 56, 57, 60, 62, 74, 81, 104, 111, 121, 151, 159, 161, 183, 186, 188, and 192. In another aspect, a variant comprises a substitution at twenty-one positions corresponding to any of positions 2, 17, 21, 28, 38, 41, 55, 56, 57, 60, 62, 74, 81, 104, 111, 121, 151, 159, 161, 183, 186, 188, and 192. In another aspect, a variant comprises a substitution at twenty-two positions corresponding to any of positions 2, 17, 21, 28, 38, 41, 55, 56, 57, 60, 62, 74, 81, 104, 111, 121, 151, 159, 161, 183, 186, 188, and 192.

In another aspect, a variant comprises a substitution at each position corresponding to positions 2, 17, 21, 28, 38, 41, 55, 56, 57, 60, 62, 74, 81, 104, 111, 121, 151, 159, 161, 183, 186, 188, and 192.

In one aspect, the variant comprises a substitution at a position corresponding to position 2. In another aspect, the amino acid at a position corresponding to position 2 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ile. In another aspect, the variant comprises the substitution V2I of the mature polypeptide of SEQ ID NO: 2 or SEQ ID NO: 4.

In another aspect, the variant comprises a substitution at a position corresponding to position 17. In another aspect, the amino acid at a position corresponding to position 17 is substituted with Ala, Arg, Asn, Asp, Cys, Gln, Glu, Gly, His, Ile, Leu, Lys, Met, Phe, Pro, Ser, Thr, Trp, Tyr, or Val, preferably with Leu. In another aspect, the variant comprises the substitution F17L of the mature polypeptide of SEQ ID NO: 2 or SEQ ID NO: 4.

In another aspect, the variant comprises a substitution at a position corresponding to position 21. In another aspect, the amino acid at a position corresponding to position 21 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ser. In another aspect, the variant comprises the substitution A21S of the mature polypeptide of SEQ ID NO: 2 or SEQ ID NO: 4.

In another aspect, the variant comprises a substitution at a position corresponding to position 28. In another aspect, the amino acid at a position corresponding to position 28 is substituted with Ala, Arg, Asn, Asp, Cys, Gln, Glu, Gly, His, Ile, Leu, Lys, Met, Phe, Pro, Ser, Thr, Trp, Tyr, or Val, preferably with Val. In another aspect, the variant comprises the substitution E28V of the mature polypeptide of SEQ ID NO: 2 or SEQ ID NO: 4.

In another aspect, the variant comprises a substitution at a position corresponding to position 38. In another aspect, the amino acid at a position corresponding to position 38 is substituted with Ala, Arg, Asn, Asp, Cys, Gln, Glu, Gly, His, Ile, Leu, Lys, Met, Phe, Pro, Ser, Thr, Trp, Tyr, or Val, preferably with Tyr or Phe. In another aspect, the variant comprises the substitution S38Y,F of the mature polypeptide of SEQ ID NO: 2 or SEQ ID NO: 4.

In another aspect, the variant comprises a substitution at a position corresponding to position 41. In another aspect, the amino acid at a position corresponding to position 41 is substituted with Ala, Arg, Asn, Asp, Cys, Gln, Glu, Gly, His, Ile, Leu, Lys, Met, Phe, Pro, Ser, Thr, Trp, Tyr, or Val, preferably with Asp. In another aspect, the variant comprises the substitution N41 D of the mature polypeptide of SEQ ID NO: 2 or SEQ ID NO: 4.

In another aspect, the variant comprises a substitution at a position corresponding to position 55. In another aspect, the amino acid at a position corresponding to position 55 is substituted with Ala, Arg, Asn, Asp, Cys, Gln, Glu, Gly, His, Ile, Leu, Lys, Met, Phe, Pro, Ser, Thr, Trp, Tyr, or Val, preferably with Asp. In another aspect, the variant comprises the substitution G55D of the mature polypeptide of SEQ ID NO: 2 or SEQ ID NO: 4.

In another aspect, the variant comprises a substitution at a position corresponding to position 56. In another aspect, the amino acid at a position corresponding to position 56 is substituted with Ala, Arg, Asn, Asp, Cys, Gln, Glu, Gly, His, Ile, Leu, Lys, Met, Phe, Pro, Ser, Thr, Trp, Tyr, or Val, preferably with His or Pro. In another aspect, the variant comprises the substitution R56H,P of the mature polypeptide of SEQ ID NO: 2 or SEQ ID NO: 4.

In another aspect, the variant comprises a substitution at a position corresponding to position 57. In another aspect, the amino acid at a position corresponding to position 57 is substituted with Ala, Arg, Asn, Asp, Cys, Gln, Glu, Gly, His, Ile, Leu, Lys, Met, Phe, Pro, Ser, Thr, Trp, Tyr, or Val, preferably with His. In another aspect, the variant comprises the substitution R57H of the mature polypeptide of SEQ ID NO: 2 or SEQ ID NO: 4.

In another aspect, the variant comprises a substitution at a position corresponding to position 60. In another aspect, the amino acid at a position corresponding to position 60 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ser. In another aspect, the variant comprises the substitution T60S of the mature polypeptide of SEQ ID NO: 2 or SEQ ID NO: 4.

In another aspect, the variant comprises a substitution at a position corresponding to position 62. In another aspect, the amino acid at a position corresponding to position 62 is substituted with Ala, Arg, Asn, Asp, Cys, Gln, Glu, Gly, His, Ile, Leu, Lys, Met, Phe, Pro, Ser, Thr, Trp, Tyr, or Val, preferably with Thr. In another aspect, the variant comprises the substitution S62T of the mature polypeptide of SEQ ID NO: 2 or SEQ ID NO: 4.

In another aspect, the variant comprises a substitution at a position corresponding to position 74. In another aspect, the amino acid at a position corresponding to position 74 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ala or Ser. In another aspect, the variant comprises the substitution T74A,S of the mature polypeptide of SEQ ID NO: 2 or SEQ ID NO: 4.

In another aspect, the variant comprises a substitution at a position corresponding to position 81. In another aspect, the amino acid at a position corresponding to position 81 is substituted with Ala, Arg, Asn, Asp, Cys, Gln, Glu, Gly, His, Ile, Leu, Lys, Met, Phe, Pro, Ser, Thr, Trp, Tyr, or Val, preferably with Asp. In another aspect, the variant comprises the substitution N81D of the mature polypeptide of SEQ ID NO: 2 or SEQ ID NO: 4.

In another aspect, the variant comprises a substitution at a position corresponding to position 104. In another aspect, the amino acid at a position corresponding to position 104 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ser. In another aspect, the variant comprises the substitution T104S of the mature polypeptide of SEQ ID NO: 2 or SEQ ID NO: 4.

In another aspect, the variant comprises a substitution at a position corresponding to position 111. In another aspect, the amino acid at a position corresponding to position 111 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ile. In another aspect, the variant comprises the substitution T111I of the mature polypeptide of SEQ ID NO: 2 or SEQ ID NO: 4.

In another aspect, the variant comprises a substitution at a position corresponding to position 121. In another aspect, the amino acid at a position corresponding to position 121 is substituted with Ala, Arg, Asn, Asp, Cys, Gln, Glu, Gly, His, Ile, Leu, Lys, Met, Phe, Pro, Ser, Thr, Trp, Tyr, or Val, preferably with Tyr. In another aspect, the variant comprises the substitution N121Y of the mature polypeptide of SEQ ID NO: 2 or SEQ ID NO: 4.

In another aspect, the variant comprises a substitution at a position corresponding to position 151. In another aspect, the amino acid at a position corresponding to position 151 is substituted with Ala, Arg, Asn, Asp, Cys, Gln, Glu, Gly, His, Ile, Leu, Lys, Met, Phe, Pro, Ser, Thr, Trp, Tyr, or Val, preferably with Asp. In another aspect, the variant comprises the substitution N151 D of the mature polypeptide of SEQ ID NO: 2 or SEQ ID NO: 4.

In another aspect, the variant comprises a substitution at a position corresponding to position 159. In another aspect, the amino acid at a position corresponding to position 159 is substituted with Ala, Arg, Asn, Asp, Cys, Gln, Glu, Gly, His, Ile, Leu, Lys, Met, Phe, Pro, Ser, Thr, Trp, Tyr, or Val, preferably with Arg. In another aspect, the variant comprises the substitution H159R of the mature polypeptide of SEQ ID NO: 2 or SEQ ID NO: 4.

In another aspect, the variant comprises a substitution at a position corresponding to position 161. In another aspect, the amino acid at a position corresponding to position 161 is substituted with Ala, Arg, Asn, Asp, Cys, Gln, Glu, Gly, His, Ile, Leu, Lys, Met, Phe, Pro, Ser, Thr, Trp, Tyr, or Val, preferably with Leu. In another aspect, the variant comprises the substitution M161L of the mature polypeptide of SEQ ID NO: 2 or SEQ ID NO: 4.

In another aspect, the variant comprises a substitution at a position corresponding to position 183. In another aspect, the amino acid at a position corresponding to position 183 is substituted with Ala, Arg, Asn, Asp, Cys, Gln, Glu, Gly, His, Ile, Leu, Lys, Met, Phe, Pro, Ser, Thr, Trp, Tyr, or Val, preferably with Asp. In another aspect, the variant comprises the substitution N183D of the mature polypeptide of SEQ ID NO: 2 or SEQ ID NO: 4.

In another aspect, the variant comprises a substitution at a position corresponding to position 186. In another aspect, the amino acid at a position corresponding to position 186 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ile or Val. In another aspect, the variant comprises the substitution L186I,V of the mature polypeptide of SEQ ID NO: 2 or SEQ ID NO: 4.

In another aspect, the variant comprises a substitution at a position corresponding to position 188. In another aspect, the amino acid at a position corresponding to position 188 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ala. In another aspect, the variant comprises the substitution T188A of the mature polypeptide of SEQ ID NO: 2 or SEQ ID NO: 4.

In another aspect, the variant comprises a substitution at a position corresponding to position 192. In another aspect, the amino acid at a position corresponding to position 192 is substituted with Ala, Arg, Asn, Asp, Cys, Gln, Glu, Gly, His, Ile, Leu, Lys, Met, Phe, Pro, Ser, Thr, Trp, Tyr, or Val, preferably with Asp. In another aspect, the variant comprises the substitution G192D of the mature polypeptide of SEQ ID NO: 2 or SEQ ID NO: 4.

In another aspect, the variant comprises substitutions at positions corresponding to positions 2 and 57, such as those described above.

In another aspect, the variant comprises substitutions at positions corresponding to positions 2 and 74, such as those described above.

In another aspect, the variant comprises substitutions at positions corresponding to positions 17 and 81, such as those described above.

In another aspect, the variant comprises substitutions at positions corresponding to positions 17 and 161, such as those described above.

In another aspect, the variant comprises substitutions at positions corresponding to positions 38 and 104, such as those described above.

In another aspect, the variant comprises a substitution at positions corresponding to positions 38 and 186, such as those described above.

In another aspect, the variant comprises substitutions at positions corresponding to positions 38 and 192, such as those described above.

In another aspect, the variant comprises substitutions at positions corresponding to positions 56 and 60, such as those described above.

In another aspect, the variant comprises substitutions at positions corresponding to positions 74 and 186, such as those described above.

In another aspect, the variant comprises substitutions at positions corresponding to positions 2, 74, and 186, such as those described above.

In another aspect, the variant comprises substitutions at positions corresponding to positions 17, 81, and 188, such as those described above.

In another aspect, the variant comprises substitutions at positions corresponding to positions 21, 74, and 186, such as those described above.

In another aspect, the variant comprises substitutions at positions corresponding to positions 28, 56, and 183, such as those described above.

In another aspect, the variant comprises substitutions at positions corresponding to positions 38, 74, and 186, such as those described above.

In another aspect, the variant comprises substitutions at positions corresponding to positions 41, 74, and 186, such as those described above.

In another aspect, the variant comprises substitutions at positions corresponding to positions 55, 74, and 186, such as those described above.

In another aspect, the variant comprises substitutions at positions corresponding to positions 57, 74, and 186, such as those described above.

In another aspect, the variant comprises substitutions at positions corresponding to positions 62, 74, and 186, such as those described above.

In another aspect, the variant comprises substitutions at positions corresponding to positions 74, 81, and 186, such as those described above.

In another aspect, the variant comprises substitutions at positions corresponding to positions 2, 74, 159, and 186, such as those described above.

In another aspect, the variant comprises substitutions at positions corresponding to positions 2, 17, 74, and 186, such as those described above.

In another aspect, the variant comprises substitutions at positions corresponding to positions 2, 62, 74, and 186, such as those described above.

In another aspect, the variant comprises substitutions at positions corresponding to positions 2, 74, 81, and 186, such as those described above.

In another aspect, the variant comprises substitutions at positions corresponding to positions 2, 57, 74, and 186, such as those described above.

In another aspect, the variant comprises substitutions at positions corresponding to positions 21, 74, 81, and 186, such as those described above.

In another aspect, the variant comprises substitutions at positions corresponding to positions 21, 38, 74, and 186, such as those described above.

In another aspect, the variant comprises substitutions at positions corresponding to positions 21, 55, 74, and 186, such as those described above.

In another aspect, the variant comprises substitutions at positions corresponding to positions 21, 62, 74, and 186, such as those described above.

In another aspect, the variant comprises substitutions at positions corresponding to positions 17, 74, 81, 186, and 188, such as those described above.

In another aspect, the variant comprises substitutions at positions corresponding to positions 21, 38, 74, 81, and 186, such as those described above.

In another aspect, the variant comprises substitutions at positions corresponding to positions 21, 62, 74, 81, and 186, such as those described above.

In another aspect, the variant comprises substitutions at positions corresponding to positions 21, 38, 62, 74, and 186, such as those described above.

In another aspect, the variant comprises substitutions at positions corresponding to positions 21, 55, 74, 81, and 186, such as those described above.

In another aspect, the variant comprises substitutions at positions corresponding to positions 21, 38, 55, 74, and 186, such as those described above.

In another aspect, the variant comprises substitutions at positions corresponding to positions 21, 55, 62, 74, and 186, such as those described above.

In another aspect, the variant comprises substitutions at positions corresponding to positions 28, 38, 74, 121, 151, and 186, such as those described above.

In another aspect, the variant comprises substitutions at positions corresponding to positions 21, 38, 62, 74, 81, and 186, such as those described above.

In another aspect, the variant comprises substitutions at positions corresponding to positions 21, 38, 55, 74, 81, and 186, such as those described above.

In another aspect, the variant comprises substitutions at positions corresponding to positions 21, 38, 55, 62, 74, and 186, such as those described above.

In another aspect, the variant comprises substitutions at positions corresponding to positions 21, 55, 62, 74, 81, and 186, such as those described above.

In another aspect, the variant comprises substitutions at positions corresponding to positions 2, 28, 38, 62, 74, 111, and 186, such as those described above.

In another aspect, the variant comprises substitutions at positions corresponding to positions 21, 38, 55, 62, 74, 81, and 186, such as those described above.

In another aspect, the variant comprises substitutions at positions corresponding to positions 2, 17, 21, 28, 38, 41, 55, 56, 57, 60, 62, 74, 81, 104, 111, 121, 151, 159, 161, 183, 186, 188, and 192, such as those described above.

In another aspect, the variant comprises one or more (several) substitutions selected from the group consisting of V2I, F17L, A21S, E28V, S38Y,F, N41D, G55D, R56H,P, R57H, T60S, S62T, T74A,S, N81D, T104S, T111I, N121Y, N151D, H159R, M161L, N183D, L186I,V, T188A, and G192D.

In a preferred aspect, the variant comprises the substitutions V2I+R57H of the mature polypeptide of SEQ ID NO: 2 or SEQ ID NO: 4.

In another preferred aspect, the variant comprises the substitutions V2I+T74A of the mature polypeptide of SEQ ID NO: 2 or SEQ ID NO: 4.

In another preferred aspect, the variant comprises the substitutions V2I+T74S of the mature polypeptide of SEQ ID NO: 2 or SEQ ID NO: 4.

In another preferred aspect, the variant comprises the substitutions F17L+N81D of the mature polypeptide of SEQ ID NO: 2 or SEQ ID NO: 4.

In another preferred aspect, the variant comprises the substitutions F17L+M161L of the mature polypeptide of SEQ ID NO: 2 or SEQ ID NO: 4.

In another preferred aspect, the variant comprises the substitutions S38Y+T104S of the mature polypeptide of SEQ ID NO: 2 or SEQ ID NO: 4.

In another preferred aspect, the variant comprises the substitutions S38Y+L186V of the mature polypeptide of SEQ ID NO: 2 or SEQ ID NO: 4.

In another preferred aspect, the variant comprises the substitutions S38F+G192D of the mature polypeptide of SEQ ID NO: 2 or SEQ ID NO: 4.

In another preferred aspect, the variant comprises the substitutions R56P+T60S of the mature polypeptide of SEQ ID NO: 2 or SEQ ID NO: 4.

In another preferred aspect, the variant comprises the substitutions T74S+L186V of the mature polypeptide of SEQ ID NO: 2 or SEQ ID NO: 4.

In another preferred aspect, the variant comprises the substitutions T74S+L186I of the mature polypeptide of SEQ ID NO: 2 or SEQ ID NO: 4.

In another preferred aspect, the variant comprises the substitutions T74A+L186V of the mature polypeptide of SEQ ID NO: 2 or SEQ ID NO: 4.

In another preferred aspect, the variant comprises the substitutions T74A+L186I of the mature polypeptide of SEQ ID NO: 2 or SEQ ID NO: 4.

In another preferred aspect, the variant comprises the substitutions V2I+T74S+L186V of the mature polypeptide of SEQ ID NO: 2 or SEQ ID NO: 4.

In another aspect, the variant comprises the substitutions F17L+N81D+T188A of the mature polypeptide of SEQ ID NO: 2 or SEQ ID NO: 4.

In another aspect, the variant comprises the substitutions A21S+T74S+L186V of the mature polypeptide of SEQ ID NO: 2 or SEQ ID NO: 4.

In another aspect, the variant comprises the substitutions E28V+R56H+N183D of the mature polypeptide of SEQ ID NO: 2 or SEQ ID NO: 4.

In another aspect, the variant comprises the substitutions S38Y+T74S+L186V of the mature polypeptide of SEQ ID NO: 2 or SEQ ID NO: 4.

In another aspect, the variant comprises the substitutions N41 D+T74S+L186V of the mature polypeptide of SEQ ID NO: 2 or SEQ ID NO: 4.

In another aspect, the variant comprises the substitutions G55D+T74S+L186V of the mature polypeptide of SEQ ID NO: 2 or SEQ ID NO: 4.

In another aspect, the variant comprises the substitutions R57H+ T74S+L186V of the mature polypeptide of SEQ ID NO: 2 or SEQ ID NO: 4.

In another aspect, the variant comprises the substitutions S62T+T74S+L186V of the mature polypeptide of SEQ ID NO: 2 or SEQ ID NO: 4.

In another aspect, the variant comprises the substitutions T74S+N81 D+L186V of the mature polypeptide of SEQ ID NO: 2 or SEQ ID NO: 4.

In another aspect, the variant comprises the substitutions T74A+N81 D+L186V of the mature polypeptide of SEQ ID NO: 2 or SEQ ID NO: 4.

In another aspect, the variant comprises the substitutions V2I+T74S+H159R+L186V of the mature polypeptide of SEQ ID NO: 2 or SEQ ID NO: 4.

In another aspect, the variant comprises the substitutions V2I+F17L+T74S+L1861 of the mature polypeptide of SEQ ID NO: 2 or SEQ ID NO: 4.

In another aspect, the variant comprises the substitutions V2I+S62T+T74S+L186V of the mature polypeptide of SEQ ID NO: 2 or SEQ ID NO: 4.

In another aspect, the variant comprises the substitutions V2I+T74S+N81 D+L186V of the mature polypeptide of SEQ ID NO: 2 or SEQ ID NO: 4.

In another aspect, the variant comprises the substitutions V2I+R57H+ T74S+L186V of the mature polypeptide of SEQ ID NO: 2 or SEQ ID NO: 4.

In another aspect, the variant comprises the substitutions A21S+T74S+N81D+

L186V of the mature polypeptide of SEQ ID NO: 2 or SEQ ID NO: 4.

In another aspect, the variant comprises the substitutions A21S+S38Y+T74S+L186V of the mature polypeptide of SEQ ID NO: 2 or SEQ ID NO: 4.

In another aspect, the variant comprises the substitutions A21S+G55D+T74S+L186V of the mature polypeptide of SEQ ID NO: 2 or SEQ ID NO: 4.

In another aspect, the variant comprises the substitutions A21S+S62T+T74S+L186V of the mature polypeptide of SEQ ID NO: 2 or SEQ ID NO: 4.

In another aspect, the variant comprises the substitutions F17L+T74S+N81D+L186V+T188A of the mature polypeptide of SEQ ID NO: 2 or SEQ ID NO: 4.

In another aspect, the variant comprises the substitutions A21S+S38Y+T74S+N81 D+L186V of the mature polypeptide of SEQ ID NO: 2 or SEQ ID NO: 4.

In another aspect, the variant comprises the substitutions A21S+S62Y+T74S+N81 D+L186V of the mature polypeptide of SEQ ID NO: 2 or SEQ ID NO: 4.

In another aspect, the variant comprises the substitutions A21S+S38Y+S62T+T74S+L186V of the mature polypeptide of SEQ ID NO: 2 or SEQ ID NO: 4.

In another aspect, the variant comprises the substitutions A21S+G55D+T74S+N81 D+L186V of the mature polypeptide of SEQ ID NO: 2 or SEQ ID NO: 4.

In another aspect, the variant comprises the substitutions A21S+S38Y+G55D+T74S+L186V of the mature polypeptide of SEQ ID NO: 2 or SEQ ID NO: 4.

In another aspect, the variant comprises the substitutions A21S+G55D+S62T+T74S+L186V of the mature polypeptide of SEQ ID NO: 2 or SEQ ID NO: 4.

In another aspect, the variant comprises the substitutions E28V+S38Y+T74S+

N121Y+N151 D+L186V of the mature polypeptide of SEQ ID NO: 2 or SEQ ID NO: 4.

In another aspect, the variant comprises the substitutions A21S+S38Y+S62T+T74S+N81D+L186V of the mature polypeptide of SEQ ID NO: 2 or SEQ ID NO: 4.

In another aspect, the variant comprises the substitutions A21S+S38Y+G55D+T74S+N81D+L186V of the mature polypeptide of SEQ ID NO: 2 or SEQ ID NO: 4.

In another aspect, the variant comprises the substitutions A21S+S38Y+G55D+S62T+T74S+L186V of the mature polypeptide of SEQ ID NO: 2 or SEQ ID NO: 4.

In another aspect, the variant comprises the substitutions A21S+G55D+S62T+T74S+N81D+L186V of the mature polypeptide of SEQ ID NO: 2 or SEQ ID NO: 4.

In another aspect, the variant comprises the substitutions V2I+E28V+S38Y+S62T+T74S+T111I+L186V of the mature polypeptide of SEQ ID NO: 2 or SEQ ID NO: 4.

In another aspect, the variant comprises the substitutions A21S+S38Y+G55D+S62T+T74S+N81 D+L186V of the mature polypeptide of SEQ ID NO: 2 or SEQ ID NO: 4.

The variants may further comprise an alteration, e.g., a substitution, deletion, or insertion, at one or more (several) other positions. For example, the variants may further comprise a substitution at one or more (several) positions corresponding to positions 19, 23, 84, and 88.

In one aspect, the number of further substitutions in the variants of the present invention is 1-4, such as 1, 2, 3, or 4 substitutions.

In one aspect, a variant further comprises a substitution at one or more (several) positions corresponding to any of positions 19, 23, 84, and 88. In another aspect, a variant further comprises a substitution at two positions corresponding to any of positions 19, 23, 84, and 88. In another aspect, a variant further comprises a substitution at three positions corresponding to any of positions 19, 23, 84, and 88. In another aspect, a variant further comprises a substitution at positions corresponding to positions 19, 23, 84, and 88.

In one aspect, the variant further comprises a substitution at a position corresponding to position 19. In another aspect, the amino acid at a position corresponding to position 19 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ala. In another aspect, the variant further comprises the substitution T19A of the mature polypeptide of SEQ ID NO: 2 or SEQ ID NO: 4.

In another aspect, the variant further comprises a substitution at a position corresponding to position 23. In another aspect, the amino acid at a position corresponding to position 23 is substituted with Ala, Arg, Asn, Asp, Cys, Gln, Glu, Gly, His, Ile, Leu, Lys, Met, Phe, Pro, Ser, Thr, Trp, Tyr, or Val, preferably with Pro. In another aspect, the variant further comprises the substitution G23P of the mature polypeptide of SEQ ID NO: 2 or SEQ ID NO: 4.

In another aspect, the variant further comprises a substitution at a position corresponding to position 84. In another aspect, the amino acid at a position corresponding to position 84 is substituted with Ala, Arg, Asn, Asp, Cys, Gln, Glu, Gly, His, Ile, Leu, Lys, Met, Phe, Pro, Ser, Thr, Trp, Tyr, or Val, preferably with Pro. In another aspect, the variant further comprises the substitution V84P of the mature polypeptide of SEQ ID NO: 2 or SEQ ID NO: 4.

In another aspect, the variant further comprises a substitution at a position corresponding to position 88. In another aspect, the amino acid at a position corresponding to position 88 is substituted with Ala, Arg, Asn, Asp, Cys, Gln, Glu, Gly, His, Ile, Leu, Lys, Met, Phe, Pro, Ser, Thr, Trp, Tyr, or Val, preferably with Thr. In another aspect, the variant further comprises the substitution I88T of the mature polypeptide of SEQ ID NO: 2 or SEQ ID NO: 4.

In another aspect, the variant further comprises substitutions at positions corresponding to positions 19 and 23, such as those described above.

In another aspect, the variant further comprises substitutions at positions corresponding to positions 19 and 84, such as those described above.

In another aspect, the variant further comprises substitutions at positions corresponding to positions 19 and 88, such as those described above.

In another aspect, the variant further comprises substitutions at positions corresponding to positions 23 and 84, such as those described above.

In another aspect, the variant further comprises substitutions at positions corresponding to positions 23 and 88, such as those described above.

In another aspect, the variant further comprises substitutions at positions corresponding to positions 84 and 88, such as those described above.

In another aspect, the variant further comprises substitutions at positions corresponding to positions 19, 23, and 84, such as those described above.

In another aspect, the variant further comprises substitutions at positions corresponding to positions 19, 23, and 88, such as those described above.

In another aspect, the variant further comprises substitutions at positions corresponding to positions 19, 84, and 88, such as those described above.

In another aspect, the variant further comprises substitutions at positions corresponding to positions 23, 84, and 88, such as those described above.

In another aspect, the variant further comprises substitutions at positions corresponding to positions 19, 23, 84, and 88, such as those described above.

In another aspect, the variant further comprises one or more (several) substitutions selected from the group consisting of T19A, G23P, V84P, and I88T.

In another preferred aspect, the variant further comprises the substitutions T19A+G23P of the mature polypeptide of SEQ ID NO: 2 or SEQ ID NO: 4.

In another preferred aspect, the variant further comprises the substitutions T19A+V84P of the mature polypeptide of SEQ ID NO: 2 or SEQ ID NO: 4.

In another preferred aspect, the variant further comprises the substitutions T19A+I88T of the mature polypeptide of SEQ ID NO: 2 or SEQ ID NO: 4.

In another preferred aspect, the variant further comprises the substitutions G23P+V84P of the mature polypeptide of SEQ ID NO: 2 or SEQ ID NO: 4.

In another preferred aspect, the variant further comprises the substitutions G23P+I88T of the mature polypeptide of SEQ ID NO: 2 or SEQ ID NO: 4.

In another preferred aspect, the variant further comprises the substitutions V84P+I88T of the mature polypeptide of SEQ ID NO: 2 or SEQ ID NO: 4.

In another preferred aspect, the variant further comprises the substitutions T19A+G23P+V84P of the mature polypeptide of SEQ ID NO: 2 or SEQ ID NO: 4.

In another preferred aspect, the variant further comprises the substitutions T19A+G23P+I88T of the mature polypeptide of SEQ ID NO: 2 or SEQ ID NO: 4.

In another preferred aspect, the variant further comprises the substitutions T19A+

V84P+I88T of the mature polypeptide of SEQ ID NO: 2 or SEQ ID NO: 4.

In another preferred aspect, the variant further comprises the substitutions G23P+V84P+I88T of the mature polypeptide of SEQ ID NO: 2 or SEQ ID NO: 4.

In another preferred aspect, the variant further comprises the substitutions T19A+G23P+V84P+I88T of the mature polypeptide of SEQ ID NO: 2 or SEQ ID NO: 4.

Essential amino acids in a parent can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for xylanase activity to identify amino acid residues that are critical to the activity of the molecule. See also, Hilton et al., 1996, J. Biol. Chem. 271: 4699-4708. The active site of the xylanase or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identities of essential amino acids can also be inferred from analysis of identities with polypeptides that are related to the parent.

The variants may consist of 151 to 160, 161 to 170, 171 to 180, 181 to 190, 191 to 200, 201 to 210, 211 to 220, 221 to 230, 231 to 240, 241 to 250, 251 to 260, 261 to 270, or 271 to 280 amino acids.

### Polynucleotides

The present invention also relates to isolated polynucleotides that encode any of the variants of the present invention.

### Nucleic Acid Constructs

The present invention also relates to nucleic acid constructs comprising a polynucleotide encoding a variant of the present invention operably linked to one or more (several) control sequences that direct the expression of the coding sequence in a suitable host cell under conditions compatible with the control sequences.

A polynucleotide may be manipulated in a variety of ways to provide for expression of a variant. Manipulation of the polynucleotide prior to its insertion into a vector may be desirable or necessary depending on the expression vector. The techniques for modifying polynucleotides utilizing recombinant DNA methods are well known in the art.

The control sequence may be a promoter sequence, a polynucleotide recognized by a host cell for expression of the polynucleotide encoding a variant of the present invention. The promoter sequence contains transcriptional control sequences that mediate the expression of the variant. The promoter may be any polynucleotide that shows transcriptional activity in the host cell of choice including mutant, truncated, and hybrid promoters, and may be obtained from genes encoding extracellular or intracellular polypeptides either homologous or heterologous to the host cell.

Examples of suitable promoters for directing the transcription of the nucleic acid constructs of the present invention in a bacterial host cell are the promoters obtained from the Bacillus amyloliquefaciens alpha-amylase gene (amyQ), Bacillus licheniformis alpha-amylase gene (31 myl), Bacillus licheniformis penicillinase gene (penP), Bacillus stearothermophilus maltogenic amylase gene (amyM), Bacillus subtilis levansucrase gene (sacB), Bacillus subtilis xylA and xylB genes, E. coli lac operon, E. coli trc promoter (Egon et al., 1988, Gene 69: 301-315), Streptomyces coelicolor agarase gene (dagA), and prokaryotic beta-lactamase gene (VIIIa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra.

Examples of suitable promoters for directing the transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Dania (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a modified promoter from a gene encoding a neutral alpha-amylase in Aspergilli in which the untranslated leader has been replaced by an untranslated leader from a gene encoding triose phosphate isomerase in Aspergilli; non-limiting examples include modified promoters from the gene encoding neutral alpha-amylase in Aspergillus niger in which the untranslated leader has been replaced by an untranslated leader from the gene encoding triose phosphate isomerase in Aspergillus nidulans or Aspergillus oryzae); and mutant, truncated, and hybrid promoters thereof.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/31myl31chyma31ydes-3-phosphate dehydrogenase (AD H1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a suitable transcription terminator sequence, which is recognized by a host cell of choice to terminate transcription. The terminator sequence is operably linked to the 3′-terminus of the polynucleotide encoding the variant. Any terminator that is functional in the host cell may be used in the present invention.

Preferred terminators for filamentous fungal host cells are obtained from the genes for Aspergillus nidulans anthranilate synthase, Aspergillus niger alpha-glucosidase, Aspergillus niger glucoamylase, Aspergillus oryzae TAKA amylase, and Fusarium oxysporum trypsin-like protease.

Preferred terminators for yeast host cells are obtained from the genes for Saccharomyces cerevisiae enolase, Saccharomyces cerevisiae cytochrome C(CYC1), and Saccharomyces cerevisiae 32myl32chyma32ydes-3-phosphate dehydrogenase. Other useful terminators for yeast host cells are described by Romanos et al., 1992, supra.

The control sequence may also be a suitable leader sequence, when transcribed is a nontranslated region of an mRNA that is important for translation by the host cell. The leader sequence is operably linked to the 5′-terminus of the polynucleotide encoding the variant. Any leader sequence that is functional in the host cell of choice may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/32myl32chyma32ydes-3-phosphate dehydrogenase (ADH2/GAP).

The control sequence may also be a polyadenylation sequence, a sequence operably linked to the 3′-terminus of the variant-encoding sequence and, when transcribed, is recognized by the host cell as a signal to add polyadenosine residues to transcribed mRNA. Any polyadenylation sequence that is functional in the host cell of choice may be used.

Preferred polyadenylation sequences for filamentous fungal host cells are obtained from the genes for Aspergillus niger glucoamylase, Aspergillus niger alpha-glucosidase, Aspergillus nidulans anthranilate synthase, Aspergillus oryzae TAKA amylase, and Fusarium oxysporum trypsin-like protease.

Useful polyadenylation sequences for yeast host cells are described by Guo and Sherman, 1995, Mol. Cellular. Biol. 15: 5983-5990.

The control sequence may also be a signal peptide coding region that encodes a signal peptide linked to the N-terminus of a variant and directs the variant into the cell's secretory pathway. The 5′-end of the coding sequence of the polynucleotide may inherently contain a signal peptide coding sequence naturally linked in translation reading frame with the segment of the coding sequence that encodes the variant. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, the foreign signal peptide coding sequence may simply replace the natural signal peptide coding sequence in order to enhance secretion of the variant. However, any signal peptide coding sequence that directs the expressed variant into the secretory pathway of a host cell of choice may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens 33myl33chym, Humicola insolens endoglucanase V, Humicola 33myl33chyma lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a variant. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophile laccase (WO 95/33836), Rhizomucor miehei aspartic proteinase, and Saccharomyces cerevisiae alpha-factor.

Where both signal peptide and propeptide sequences are present at the N-terminus of a variant, the propeptide sequence is positioned next to the N-terminus of the variant and the signal peptide sequence is positioned next to the N-terminus of the propeptide sequence.

It may also be desirable to add regulatory sequences that allow the regulation of the expression of the variant relative to the growth of the host cell. Examples of regulatory systems are those that cause the expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the variant would be operably linked with the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the variant at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about the expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are the dal genes from Bacillus licheniformis or Bacillus subtilis, or markers that confer antibiotic resistance such as ampicillin, chloramphenicol, kanamycin, or tetracycline resistance. Suitable markers for yeast host cells are ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are the amdS and pyrG genes of Aspergillus nidulans or Aspergillus oryzae and the bar gene of Streptomyces hygroscopicus.

The vector preferably contains an element(s) that permits integration of the vector into the host cell's genome or autonomous replication of the vector in the cell independent of the genome.

For integration into the host cell genome, the vector may rely on the polynucleotide's sequence encoding the variant or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination.

The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAM131 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANSI (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a variant. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide of the present invention operably linked to one or more control sequences that direct the production of a variant of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the variant and its source.

The host cell may be any cell useful in the recombinant production of a variant of the present invention, e.g., a prokaryote or a eukaryote.

The prokaryotic host cell may be any gram-positive or gram-negative bacterium. Gram-positive bacteria include, but not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), by using competent cells (see, e.g., Young and Spizizen, 1961, J. Bacteriol. 81: 823-829, or Dubnau and Davidoff-Abelson, 1971, J. Mol. Biol. 56: 209-221), by electroporation (see, e.g., Shigekawa and Dower, 1988, Biotechniques 6: 742-751), or by conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation and electroporation (see, e.g., Gong et al., 2004, Folia Microbiol. (Praha) 49: 399-405), by conjugation (see, e.g., Mazodier et al., 1989, J. Bacteriol. 171: 3583-3585), or by transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or by conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), by protoplast transformation (see, e.g., Catt and Jollick, 1991, Microbios 68: 189-207, by electroporation (see, e.g., Buckley et al., 1999, Appl. Environ. Microbiol. 65: 3800-3804) or by conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota (as defined by Hawksworth et al., In, Ainsworth and Bisby's Dictionary of The Fungi, 8^{th} edition, 1995, CAB International, University Press, Cambridge, UK) as well as the Oomycota (as cited in Hawksworth et al., 1995, supra, page 171) and all mitosporic fungi (Hawksworth et al., 1995, supra).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, F. A., Passmore, S. M., and Davenport, R. R., eds, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a 38myl38ch wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola 39myl39chyma, Mucor miehei, Myceliophthora thermophile, Neurospora crassa, Penicillium purpurogenum, Phanerochaete chrysosporium, Phlebia 39myl39ch, Pleurotus eryngii, Thielavia terrestris, Trametes villosa, Trametes versicolor, Trichoderma harzianum, Trichoderma Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023 and Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

The present invention also relates to methods of producing a variant, comprising: (a) cultivating a host cell of the present invention under conditions suitable for the expression of the variant; and (b) recovering the variant.

The host cells are cultivated in a nutrient medium suitable for production of the variant using methods well known in the art. For example, the cell may be cultivated by shake flask cultivation, and small-scale or large-scale fermentation (including continuous, batch, fed-batch, or solid state fermentations) in laboratory or industrial fermentors performed in a suitable medium and under conditions allowing the variant to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the variant is secreted into the nutrient medium, the variant can be recovered directly from the medium. If the variant is not secreted, it can be recovered from cell lysates.

The variant may be detected using methods known in the art that are specific for the variants. These detection methods may include use of specific antibodies, formation of an enzyme product, or disappearance of an enzyme substrate. For example, an enzyme assay may be used to determine the activity of the variant.

The variant may be recovered using methods known in the art. For example, the variant may be recovered from the nutrient medium by conventional procedures including, but not limited to, centrifugation, filtration, extraction, spray-drying, evaporation, or precipitation.

The variant may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J.-C. Janson and Lars Ryden, editors, VCH Publishers, New York, 1989) to obtain substantially pure variants.

In an alternative aspect, the variant is not recovered, but rather a host cell of the present invention expressing the variant is used as a source of the variant.

### Compositions

The present invention also relates to compositions comprising a variant of the present invention. Preferably, the compositions are enriched in such a variant. The term “enriched” means that the xylanase activity of the composition has been increased, e.g., with an enrichment factor of 1.1.

The composition may comprise a variant as the major enzymatic component, e.g., a mono-component composition. Alternatively, the composition may comprise multiple enzymatic activities, such as an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, 40myl-40chym, chitinase, cutinase, cyclodextrin glycosyltransferase, deoxyribonuclease, endoglucanase, esterase, glucoamylase, haloperoxidase, invertase, laccase, lipase, mannosidase, oxidase, pectinolytic enzyme, peptidoglutaminase, peroxidase, phytase, polyphenoloxidase, proteolytic enzyme, ribonuclease, transglutaminase, or xylanase. The additional enzyme(s) may be produced, for example, by a microorganism belonging to the genus Aspergillus, e.g., Aspergillus aculeatus, Aspergillus awamori, Aspergillus foetidus, Aspergillus fumigatus, Aspergillus japonicus, Aspergillus nidulans, Aspergillus niger, or Aspergillus oryzae; Fusarium, e.g., Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sulphureum, Fusarium toruloseum, Fusarium trichothecioides, or Fusarium venenatum; Humicola, e.g., Humicola insolens or Humicola 40myl40chyma; or Trichoderma, e.g., Trichoderma harzianum, Trichoderma koningii, Trichoderma longibrachiatum, Trichoderma reesei, or Trichoderma viride. The compositions may be prepared in accordance with methods known in the art and may be in the form of a liquid or a dry composition. For instance, the composition may be in the form of a granulate or microgranulate. The variant may be stabilized in accordance with methods known in the art.

Examples are given below of preferred uses of the compositions of the invention. The dosage of the composition of the invention and other conditions under which the composition is used may be determined on the basis of methods known in the art.

### Uses

A variant of the present invention may be used in several applications to degrade or convert a xylan-containing material comprising treating the material with the variant (see, for example, WO 2002/18561). A variant of the present invention may be used to enhance the brightness of pulp, to improve the quality of paper, to decrease the amount of chemical bleaching agents such as chlorine used in the pulp bleaching stages, and to treat pulp for other purposes, without inducing any damage of cellulose in pulp. The variants may be used in methods for the treatment of pulp, e.g., Kraft pulp, according to U.S. Pat. No. 5,658,765. Pulp is a dry fibrous material prepared by chemically or mechanically separating fibers from wood, fiber crops, or waste paper. Wood pulp is the most common material used to make paper. The timber resources used to make wood pulp are referred to as pulpwood. Wood pulp comes from softwood trees such as spruce, pine, fir, larch, and hemlock, and hardwoods such as eucalyptus, aspen, and birch.

The variants can be used in bleaching of pulp to reduce the use of toxic chlorine-containing chemicals. In addition, it is desirable that xylanases used for biobleaching are stable and active under alkaline conditions at high temperatures. In a preferred embodiment, the present invention relates to methods for treating a pulp, comprising contacting the pulp with the variant.

In the pulp treatment according to the present invention, conditions of the enzymes for treating pulp, such as temperature, pH, pressure, time period, etc., may be suitably chosen so that the desired enzymatic action is exhibited to achieve the desired effects such as enhancement of the brightness. For example, the temperature may be in the range of 10 to 90° C., e.g., 25 to 85° C., 30 to 85° C., 40 to 85° C., 50 to 85° C., 60 to 80° C., 70 to 80° C., or any other suitable temperature. The pH may be in the range of 3 to 11, e.g., 4 to 10, 5 to 10, 6 to 10, 7 to 10, 7 to 9.5, 8 to 9.5, or any other suitable pH. The pulp is treated with a variant in the amount of 0.1 to 25 mg/kg dry pulp, e.g., 0.25 to 20, 0.5 to 10, 0.75 to 10, 1 to 8, 1 to 6, 1 to 5 mg/kg dry pulp, or any other suitable amount.

The pressure may be applied under such a pressure conventionally used for pulp bleaching or other ordinary pulp treating steps; there is no particular restriction but normal pressure is preferably from an economic standpoint. The time period for the treatments may be in the range of 10 minutes to 50 hours, e.g., 0.5 hour to 24 hours, 1 hour to 24 hours, 1 hour to 12 hours, 1 hour to 5 hours, e.g., 2 hours, or any other suitable time period.

In the case where it is desired to enhance the brightness, the amount of a chemical bleaching agent used after the enzymatic treatment can be greatly reduced. The pulp treatment of the present invention is sufficient as a substitute for at least a part of the current bleaching process using chlorine bleaching agents.

The method of the present invention for treating pulp is applicable to a wide range of pulp derived from a broadleaf tree, a needle-leaf tree, or a non-tree material, such as kraft pulp, sulfite pulp, semi-chemical pulp, groundwood pulp, refiner groundwood pulp, thermo-mechanical pulp, etc. By applying the pulp treatment method of the present invention to these pulps, the amount of lignin remaining in the pulp can be reduced to attain the effects such as enhancement of the brightness of pulps, improvement of the quality, and decrease of the amount of a chemical bleaching agent. The pulp treatment method of the present invention may also be applied to the bleaching steps of these pulps by oxygen or the like, prior to or after the bleaching.

Following the pulp treatment using a variant of the present invention, an extraction may also be carried out to effectively remove the lignin dissolved or susceptible to be dissolved out of the pulp. The extraction may be performed using, e.g., sodium hydroxide. In this case, typical conditions for the extraction are set forth to have a pulp concentration of 0.3 to 20%, a sodium hydroxide concentration of 0.5 to 5% based on the weight of dry pulp, a temperature range of 40 to 80° C., and a time period for 30 minutes to 3 hours, e.g., 1 to 2 hours. However, any suitable extraction known in the art may be used.

After the pulp is treated according to the method of the present invention, a chemical bleaching agent may also be used to further enhance the brightness of the pulp. In this case, even if the amount of the chemical bleaching agent is greatly decreased as compared to the case of bleaching pulp only with the chemical bleaching agent, a better brightness can be obtained. Where chlorine dioxide is used as a chemical bleaching agent, the amount of chlorine dioxide can be reduced by 23% to 43% or even more.

When paper is made from the pulp treated according to the method of the present invention, the paper has excellent properties such as a lower content of chlorinated phenol compounds, as compared to paper prepared from conventional bleached pulp.

The variants may also be used in processes for producing xylose or xylo-oligosaccharide according to U.S. Pat. No. 5,658,765. In another preferred embodiment, the present invention relates to methods for producing xylose, comprising contacting a xylan-containing material with the variant. In one aspect, the method further comprises recovering the xylose.

The variants may also be used as feed enhancing enzymes that improve feed digestibility to increase the efficiency of its utilization according to U.S. Pat. No. 6,245,546.

The variants may also be used in baking according to U.S. Pat. No. 5,693,518.

The variants may further be used in brewing according to WO 2002/24926.

### Plants

The present invention also relates to isolated plants, e.g., a transgenic plant, plant part, or plant cell, comprising a polynucleotide of the present invention so as to express and produce the variant in recoverable quantities. The variant may be recovered from the plant or plant part. Alternatively, the plant or plant part containing the variant may be used as such for improving the quality of a food or feed, e.g., improving nutritional value, palatability, and rheological properties, or to destroy an antinutritive factor.

The transgenic plant can be dicotyledonous (a dicot) or monocotyledonous (a monocot). Examples of monocot plants are grasses, such as meadow grass (blue grass, Poa), forage grass such as Festuca, Lolium, temperate grass, such as Agrostis, and cereals, e.g., wheat, oats, rye, barley, rice, sorghum, and maize (corn).

Examples of dicot plants are tobacco, legumes, such as lupins, potato, sugar beet, pea, bean and soybean, and cruciferous plants (family Brassicaceae), such as cauliflower, rape seed, and the closely related model organism Arabidopsis thaliana.

Examples of plant parts are stem, callus, leaves, root, fruits, seeds, and tubers as well as the individual tissues comprising these parts, e.g., epidermis, mesophyll, 43myl43chyma, vascular tissues, meristems. Specific plant cell compartments, such as chloroplasts, apoplasts, mitochondria, vacuoles, peroxisomes and cytoplasm are also considered to be a plant part. Furthermore, any plant cell, whatever the tissue origin, is considered to be a plant part. Likewise, plant parts such as specific tissues and cells isolated to facilitate the utilization of the invention are also considered plant parts, e.g., embryos, endosperms, aleurone and seed coats.

Also included within the scope of the present invention are the progeny of such plants, plant parts, and plant cells.

The transgenic plant or plant cell expressing a variant may be constructed in accordance with methods known in the art. In short, the plant or plant cell is constructed by incorporating one or more expression constructs encoding a variant into the plant host genome or chloroplast genome and propagating the resulting modified plant or plant cell into a transgenic plant or plant cell.

The expression construct is conveniently a nucleic acid construct that comprises a polynucleotide encoding a variant operably linked with appropriate regulatory sequences required for expression of the polynucleotide in the plant or plant part of choice. Furthermore, the expression construct may comprise a selectable marker useful for identifying host cells into which the expression construct has been integrated and DNA sequences necessary for introduction of the construct into the plant in question (the latter depends on the DNA introduction method to be used).

The choice of regulatory sequences, such as promoter and terminator sequences and optionally signal or transit sequences, is determined, for example, on the basis of when, where, and how the variant is desired to be expressed. For instance, the expression of the gene encoding a variant may be constitutive or inducible, or may be developmental, stage or tissue specific, and the gene product may be targeted to a specific tissue or plant part such as seeds or leaves. Regulatory sequences are, for example, described by Tague et al., 1988, Plant Physiology 86: 506.

For constitutive expression, the 35S-CaMV, the maize ubiquitin 1, or the rice actin 1 promoter may be used (Franck et al., 1980, Cell 21: 285-294; Christensen et al., 1992, Plant Mol. Biol. 18: 675-689; Zhang et al., 1991, Plant Cell 3: 1155-1165). Organ-specific promoters may be, for example, a promoter from storage sink tissues such as seeds, potato tubers, and fruits (Edwards and Coruzzi, 1990, Ann. Rev. Genet. 24: 275-303), or from metabolic sink tissues such as meristems (Ito et al., 1994, Plant Mol. Biol. 24: 863-878), a seed specific promoter such as the glutelin, prolamin, globulin, or albumin promoter from rice (Wu et al., 1998, Plant Cell Physiol. 39: 885-889), a Vicia faba promoter from the legumin B4 and the unknown seed protein gene from Vicia faba (Conrad et al., 1998, J. Plant Physiol. 152: 708-711), a promoter from a seed oil body protein (Chen et al., 1998, Plant Cell Physiol. 39: 935-941), the storage protein napA promoter from Brassica napus, or any other seed specific promoter known in the art, e.g., as described in WO 91/14772. Furthermore, the promoter may be a leaf specific promoter such as the rbcs promoter from rice or tomato (Kyozuka et al., 1993, Plant Physiol. 102: 991-1000), the chlorella virus adenine methyltransferase gene promoter (Mitra and Higgins, 1994, Plant Mol. Biol. 26: 85-93), the aldP gene promoter from rice (Kagaya et al., 1995, Mol. Gen. Genet. 248: 668-674), or a wound inducible promoter such as the potato pint promoter (Xu et al., 1993, Plant Mol. Biol. 22: 573-588). Likewise, the promoter may induced by abiotic treatments such as temperature, drought, or alterations in salinity or induced by exogenously applied substances that activate the promoter, e.g., ethanol, oestrogens, plant hormones such as ethylene, abscisic acid, and gibberellic acid, and heavy metals.

A promoter enhancer element may also be used to achieve higher expression of a variant in the plant. For instance, the promoter enhancer element may be an intron that is placed between the promoter and the polynucleotide encoding a variant. For instance, Xu et al., 1993, supra, disclose the use of the first intron of the rice actin 1 gene to enhance expression.

The selectable marker gene and any other parts of the expression construct may be chosen from those available in the art.

The nucleic acid construct is incorporated into the plant genome according to conventional techniques known in the art, including Agrobacterium-mediated transformation, virus-mediated transformation, microinjection, particle bombardment, biolistic transformation, and electroporation (Gasser et al., 1990, Science 244: 1293; Potrykus, 1990, Bio/Technology 8: 535; Shimamoto et al., 1989, Nature 338: 274).

Presently, Agrobacterium tumefaciens-mediated gene transfer is the method of choice for generating transgenic dicots (for a review, see Hooykas and Schilperoort, 1992, Plant Mol. Biol. 19: 15-38) and can also be used for transforming monocots, although other transformation methods are often used for these plants. Presently, the method of choice for generating transgenic monocots is particle bombardment (microscopic gold or tungsten particles coated with the transforming DNA) of embryonic calli or developing embryos (Christou, 1992, Plant J. 2: 275-281; Shimamoto, 1994, Curr. Opin. Biotechnol. 5: 158-162; Vasil et al., 1992, Bio/Technology 10: 667-674). An alternative method for transformation of monocots is based on protoplast transformation as described by Omirulleh et al., 1993, Plant Mol. Biol. 21: 415-428. Additional transformation methods for use in accordance with the present disclosure include those described in U.S. Pat. Nos. 6,395,966 and 7,151,204 (both of which are herein incorporated by reference in their entirety).

Following transformation, the transformants having incorporated the expression construct are selected and regenerated into whole plants according to methods well known in the art. Often the transformation procedure is designed for the selective elimination of selection genes either during regeneration or in the following generations by using, for example, co-transformation with two separate T-DNA constructs or site specific excision of the selection gene by a specific recombinase.

In addition to direct transformation of a particular plant genotype with a construct of the present invention, transgenic plants may be made by crossing a plant having the construct to a second plant lacking the construct. For example, a construct encoding a variant can be introduced into a particular plant variety by crossing, without the need for ever directly transforming a plant of that given variety. Therefore, the present invention encompasses not only a plant directly regenerated from cells which have been transformed in accordance with the present invention, but also the progeny of such plants. As used herein, progeny may refer to the offspring of any generation of a parent plant prepared in accordance with the present invention. Such progeny may include a DNA construct prepared in accordance with the present invention, or a portion of a DNA construct prepared in accordance with the present invention. Crossing results in the introduction of a transgene into a plant line by cross pollinating a starting line with a donor plant line. Non-limiting examples of such steps are further articulated in U.S. Pat. No. 7,151,204.

Plants may be generated through a process of backcross conversion. For example, plants include plants referred to as a backcross converted genotype, line, inbred, or hybrid.

Genetic markers may be used to assist in the introgression of one or more transgenes of the invention from one genetic background into another. Marker assisted selection offers advantages relative to conventional breeding in that it can be used to avoid errors caused by phenotypic variations. Further, genetic markers may provide data regarding the relative degree of elite germplasm in the individual progeny of a particular cross. For example, when a plant with a desired trait which otherwise has a non-agronomically desirable genetic background is crossed to an elite parent, genetic markers may be used to select progeny which not only possess the trait of interest, but also have a relatively large proportion of the desired germplasm. In this way, the number of generations required to introgress one or more traits into a particular genetic background is minimized.

The present invention also relates to methods of producing a variant of the present invention comprising: (a) cultivating a transgenic plant or a plant cell comprising a polynucleotide encoding the variant under conditions conducive for production of the variant; and (b) recovering the variant.

The present invention is further described by the following examples that should not be construed as limiting the scope of the invention.

### EXAMPLES

### Strains

Bacillus subtilis 168Δ4 is derived from the Bacillus subtilis type strain 168 (BGSC 1A1, Bacillus Genetic Stock Center, Columbus, Ohio, USA) and has deletions in the spoIIAC, aprE, nprE, and amyE genes. The deletion of the four genes was performed essentially as described for Bacillus subtilis A164Δ5 (U.S. Pat. No. 5,891,701).

Bacillus subtilis strain McLp2 (168Δ4, xynAΔ pel::triple promoter comprising a Bacillus licheniformis 46myl 4199 promoter having a mutation corresponding to position 5, a short consensus Bacillus amyloliquefaciens amyQ promoter having the sequence TTGACA for the “−35” region and TATAAT for the “−10” region, and a Bacillus thuringiensis subsp. Tenebrionis crylIIA promoter [WO 2003/095658], neo^{s}, spec^{R}) was used for expression of Thermobifida fusca Family 11 xylanase variants.

Bacillus subtilis strain McLp7 (164Δ5 [spoIIAC, aprE, nprE, amyE, srfAC; U.S. Pat. No. 5,891,701], xynAΔ pel::triple promoter comprising a Bacillus licheniformis 46myl 4199 promoter having a mutation corresponding to position 5, a short consensus Bacillus amyloliquefaciens amyQ promoter having the sequence TTGACA for the “−35” region and TATAAT for the “−10” region, and a Bacillus thuringiensis subsp. Tenebrionis cryIIIA promoter [WO 2003/095658] spec^{R}) was used for expression of Thermobifida fusca Family 11 xylanase variants.

### Media

Spizizen I medium was composed of 6 g of KH₂PO₄, 14 g of K₂HPO₄, 2 g of (NH₄)₂SO₄, 1 g of Na₃C₆H₅O₇, 0.2 g of MgSO₄.7H₂O, 5 g of glucose, 0.2 g of casein hydrolysate, 1 g of yeast extract, 50 mg of tryptophan, and deionized water to 1 liter.

Spizizen II medium was composed of Spizizen I medium and 0.055 g of CaCl₂ and 0.24 g of MgCl₂ per liter.

LB medium was composed of 10 g of tryptone, 5 g of yeast extract, 5 g of NaCl, and deionized water to 1 liter.

LB+Amp medium was composed of LB medium supplemented with 100 μg of ampicillin per ml.

LB agar medium was composed of 10 g of tryptone, 5 g of yeast extract, 5 g of NaCl, 15 g of bacto agar, and deionized water to 1 liter.

LB+Amp agar medium was composed of LB agar medium supplemented with 100 μg of ampicillin per ml.

LB+Cm agar medium was composed of LB agar medium supplemented with 5 μg of chloramphenicol per ml.

LB+Cm agar medium with 0.1% AZCl-xylan is composed of LB+Cm agar medium supplemented with 0.1 g of AZCl-arabinoxylan (Megazyme, Ireland) per liter.

LB plates with 0.1% AZCl-xylan were composed of 10 g of tryptone, 5 g of yeast extract, 5 g of NaCl, 15 g of Bacto agar, 1 g of AZCl-xylan birchwood (Megazyme, Ireland), and deionized water to 1 liter.

TBAB+Cm plates were composed of 33 g of TBAB (Tryptose Blood Agar Base), 5 mg of chloramphenicol, and deionized water to 1 liter.

MY25 medium was composed of 25 g of maltodextrin, 2 g of MgSO₄.7H₂O, 10 g of KH₂PO₄, 2 g of citric acid anhydrous powder, 2 g of K₂SO₄, 2 g of urea, 10 g of yeast extract, 0.5 ml of AMG trace metals solution, and deionized water to 1 liter.

AMG trace metals solution was composed of 14.3 g of ZnSO₄.7H₂O, 2.5 g of CuSO₄.5H₂O, 0.5 g of NiCl₂.6H₂O, 13.8 g of FeSO₄.7H₂O, 8.5 g of MnSO₄.7H₂O, 3 g of citric acid, and deionized water to 1 liter.

DIFCO™ Lactobacilli MRS broth was composed of 10 g of Proteose Peptone No. 3, 10 g of beef extract, 5 g of yeast extract, 20 g of dextrose, 1 g of polysorbate 80, 2 g of ammonium citrate, 5 g of sodium acetate, 0.1 g of magnesium sulfate, 0.05 g of manganese sulfate, 2 g of dipotassium phosphate, and deionized water to 1 liter.

### Example 1

### Construction of the Bacillus subtilis Plasmids pTH025 and pTH153

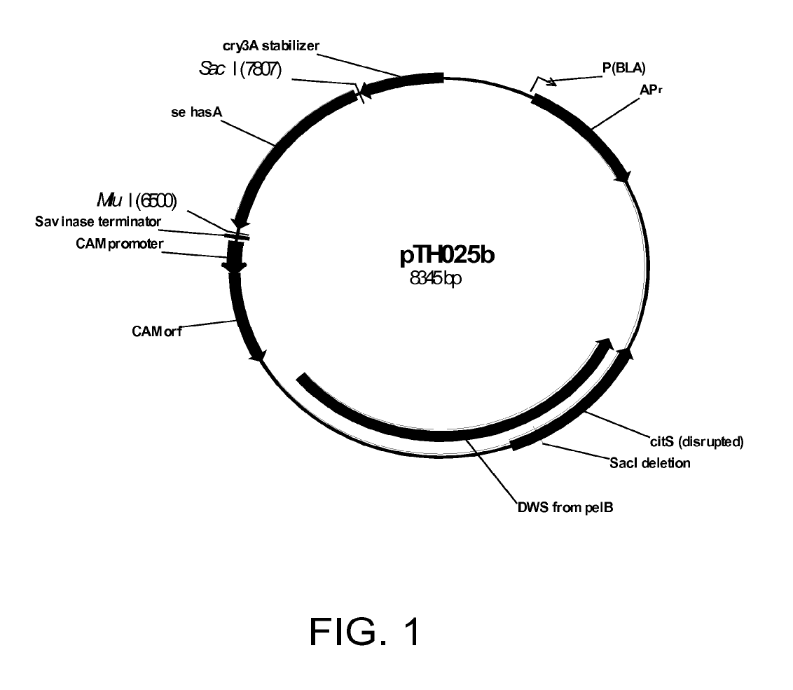

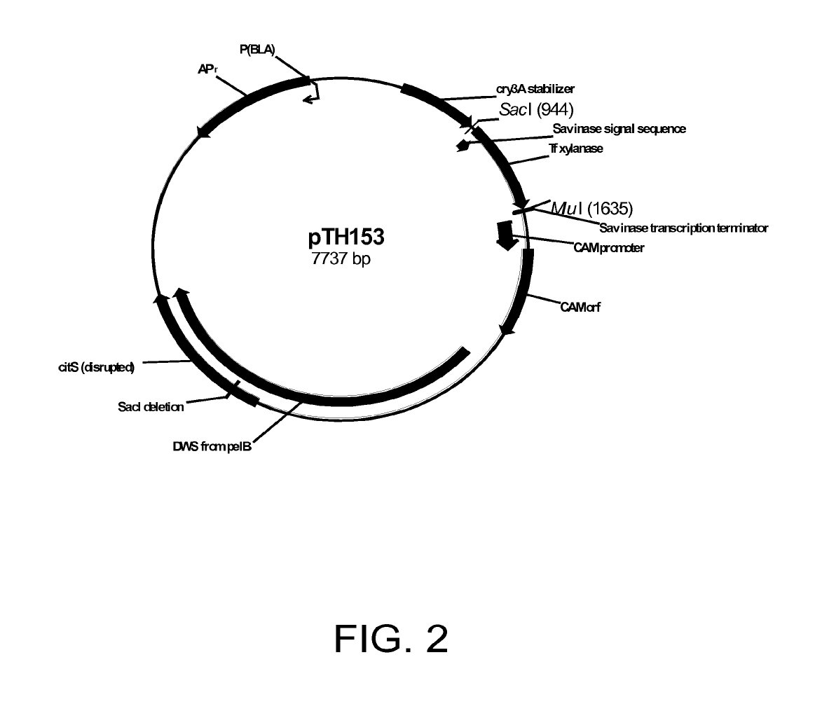

Plasmids pTH025 and pTH153 were constructed as described below. Plasmid pTH025 (FIG. 1) was constructed for integration and expression of mutant gene libraries and was also used to construct plasmid pTH153, a Bacillus subtilis integration expression vector containing a Thermobifida fusca Family 11 xylanase synthetic gene minus the cellulose binding module (CBM) region. Plasmid pTH153 (FIG. 2) was constructed for use as a positive control in library and variant screens and was also used as template for generation of error-prone random libraries.

The following steps describe the construction of pTH025. Plasmid pMB1508 (U.S. Pat. No. 7,485,447) was digested with Pac I and Kpn I to remove a 913 bp fragment containing one of two Sac I sites present on the plasmid. The 6.5 kb plasmid fragment was blunt-ended with T4 DNA polymerase (New England Biolabs, Inc., Ipswich, Mass., USA), purified by 1% agarose gel electrophoresis in 40 mM Tris base-20 mM sodium acetate-1 mM disodium EDTA (TAE) buffer, excised from the gel, extracted using a QIAQUICK® Gel Extraction Kit (QIAGEN Inc., Valencia, Calif., USA), self-ligated to recircularize the plasmid using a Rapid Ligation Kit (Roche Applied Science, Mannheim, Germany), and transformed into E. coli XL-1 Blue Sub-cloning Grade Competent cells (Stratagene, La Jolla, Calif.). Transformants were selected on LB+Amp medium. Plasmid DNA from several of the resulting E. coli transformants was prepared using a BIOROBOT® 9600 (QIAGEN Inc., Valencia, Calif., USA). Resulting plasmid pTH022 was verified by restriction enzyme digestion with Sac I and Pst I, which indicated that the correct plasmid construct contained only one Sac I site, and complete digestion of pTH022 yielded two fragments of 5 kb and 871 bp by 1′)/0 agarose gel electrophoresis in TAE buffer.

Deletion of the Sac I site in plasmid pTH022 was achieved as follows: Following Sac I digestion, the plasmid was blunt-ended with T4 DNA polymerase, self-ligated using a Rapid Ligation Kit following the manufacturer's instructions, and transformed into E. coli XL-1 Blue Sub-cloning Grade Competent Cells. Transformants were selected on LB+Amp medium. Plasmid DNA from several of the resulting E. coli transformants was prepared using a BIOROBOT® 9600. Resulting plasmid pTH023 was verified by restriction enzyme digestion with Sac I and Pst I, which indicated deletion of the Sac I site by the presence of one fragment of 6.5 kb by 1% agarose gel electrophoresis in TAE buffer.

The Streptococcus equisimilis hasA gene was obtained from plasmid pRB156 (WO 2003/054163) by digesting with Pac I, blunt-ending with T4 DNA polymerase, and digesting with Not Ito liberate a 1.9 kb fragment harboring the hasA gene, which was visualized by 0.8% agarose gel electrophoresis in TAE buffer. Plasmid pTH023 was digested with Eco RI, blunt-ended using Klenow fragment, and then digested with Not I. The 1.9 kb hasA gene fragment and the 6.4 kb pTH023 vector fragment were purified by 1% agarose gel electrophoresis in TAE buffer, excised from the gels, extracted using a QIAQUICK® Gel Extraction Kit, ligated together using a Rapid Ligation Kit, and transformed into E. coli XL1-Blue Sub-cloning Grade Competent Cells (Stratagene, La Jolla, Calif., USA). Transformants were selected on LB+Amp medium. Plasmid DNA from several of the resulting E. coli transformants was prepared using a BIOROBOT® 9600. Resulting plasmid pTH025 was verified by restriction enzyme digestion with Sac I and Bgl II, which resulted in two fragments of 6.9 kb and 1.4 kb by 1% agarose gel electrophoresis in TAE buffer. Plasmid pTH025 was used as the backbone to construct plasmid pTH153.

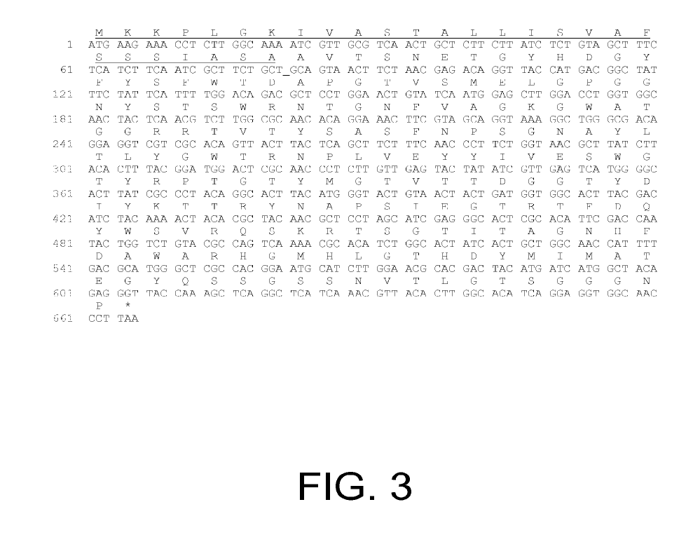

The following steps describe the construction of pTH153. A synthetic polynucleotide fragment comprising a Bacillus clausii serine protease ribosome binding site (RBS) and B. clausii serine protease signal sequence fused to a 582 bp codon-optimized gene encoding T. fusca GH11 xylanase minus the cellulose binding domain (FIG. 3) was designed to provide optimal protein expression when integrated into B. subtilis. The codon-optimized synthetic polynucleotide was synthesized by Codon Devices, Inc., (Cambridge, Mass., USA) and delivered as an E. coli derived plasmid designated ptfxyCBM. The synthetic polynucleotide was also designed to contain flanking Sac I and Mlu I restriction endonuclease sites for subsequent subcloning of T. fusca GH11 xylanase mutant polynucleotide fragments.

A 691 bp Sac I and Mlu I fragment of plasmid ptfxyCBM was subcloned into plasmid pMDT100 (WO 2008/140615) to generate plasmid intermediate pSMO248. Subcloning was accomplished by Sac I and Mlu I digestion of pMDT100 removing the B. clausii serine protease RBS fragment between these two restriction sites, purification of the remaining plasmid backbone fragment by 0.7% agarose gel electrophoresis in TAE buffer, excision from the gel, and extraction using a QIAQUICK® Gel Extraction Kit. Plasmid ptfxyCBM was digested similarly releasing the B. clausii serine protease signal sequence-Thermobifida fusca xylanase synthetic polynucleotide fragment, which was purified as above, ligated into the pMDT100 backbone using a Rapid Ligation Kit, and transformed into E. coli SURE® competent cells (Stratagene, La Jolla, Calif., USA). Transformants were selected on LB+Amp agar medium. Plasmid DNA from a single E. coli colony was isolated using a BIOROBOT® 9600 and proper insertion of the 691 bp synthetic Thermobifida fusca xylanase fragment into the pMDT100 backbone to create pSMO248 was verified by Sac I and Mlu I digestion and visualization by 1% agarose gel electrophoresis in TAE buffer.

The synthetic Thermobifida fusca xylanase polynucleotide fragment from pSMO248 was subcloned into pTH025 to generate pTH153. To achieve this, the primers shown below were designed to amplify by PCR the polynucleotide encoding the synthetic Thermobifida fusca Family 11 xylanase from pSMO248.

Forward primer (Tf.xylF):

### 5′-ATCAGTTTGAAAATTATGTATTATGGAGCTCTATAAAAATGAGGAGGG-3′ (SEQ ID NO: 5)

Reverse primer (Tf.xylR):

### 5′-CTTTAACCGCACAGCGTTTTTTTATTGATTAACGCGTTTA-3′ (SEQ ID NO: 6)

Primer Tf.xylF was designed to contain a Bacillus thuringiensis subsp. Tenebrionis cryIIIA mRNA stabilizer sequence (WO 94/25612) in addition to a 17 bp region downstream of the Sac I site on plasmid pTH025. Primer Tf.xylR was designed to contain a 31 bp region downstream of the Mlu I site on plasmid pTH025 for fusion of the PCR product and pTH025.

A total of 50 picomoles of each of the primers above were used in an amplification reaction containing 50 ng of pSMO248, 1× AMPLITAQ GOLD® Buffer II (Applied Biosystems, Foster City, Calif., USA), 1 μl of a blend of dATP, dTTP, dGTP, and dCTP, each at 10 mM, 5 units of AMPLITAQ GOLD® DNA polymerase (Applied Biosystems, Foster City, Calif., USA), and 3 ml of 25 mM MgSO₄ in a final volume of 50 μl. The amplification reaction was performed in an EPPENDORF® MASTERCYCLER® 5333 (Eppendorf EG, Hamburg, Germany) programmed for 1 cycle at 95° C. for 9 minutes; and 30 cycles each at 95° C. for 30 seconds, 55° C. for 30 seconds, and 72° C. for 30 seconds. After the 30 cycles, the reaction was heated for 5 minutes at 72° C. The heat block then went to a 10° C. soak cycle.

The reaction product was isolated by 1.0% agarose gel electrophoresis in TAE buffer where a 717 bp PCR product band was excised from the gel and extracted using a QIAQUICK® Gel Extraction Kit.

Plasmid pTH025 was gapped by digestion with Sac I and Mlu I. The digestion was verified by fractionating an aliquot of the digestion on a 0.8% agarose gel in TAE buffer where expected fragments of 7038 bp (gapped) and 1307 bp (from the Streptococcus equisimilis hasA gene) were obtained. The 7038 bp (gapped) fragment was excised from the gel and purified using a QIAQUICK® Minelute column (QIAGEN Inc., Valencia, Calif., USA).

The homologous ends of the 717 bp PCR product and plasmid pTH025, digested with Sac I and Mlu I, were joined using an IN-FUSION™ Advantage PCR Cloning Kit (Clontech Laboratories, Inc., Mountain View, Calif., USA). A total of 50 ng of the 717 bp PCR product and 100 ng of plasmid pTH025 (digested with Sac I and Mlu I) were used in a reaction containing 2 ml of 5×IN-FUSION™ reaction buffer (Clontech Laboratories, Inc., Mountain View, Calif., USA) and 1 μl of IN-FUSION™ enzyme (Clontech Laboratories, Inc., Mountain View, Calif., USA) in a final volume of 10 μl. The reaction was incubated for 15 minutes at 37° C., followed by 15 minutes at 50° C., and then placed on ice. The reaction volume was increased to 50 μl with 10 mM Tris-0.1 mM EDTA pH 8 (TE) buffer and 3 μl of the reaction were used to transform E. coli XL10-GOLD® Ultracompetent Cells (Stratagene, La Jolla, Calif., USA) according to the manufacturer's instructions. Transformants were selected on LB+Amp agar medium. Plasmid DNA from several of the resulting E. coli transformants was prepared using a BIOROBOT® 9600.

Plasmid pTH153 containing a polynucleotide encoding the B. clausii serine protease signal sequence fused to the Thermobifida fusca Family 11 xylanase synthetic gene was identified and the full-length gene sequence was determined using a 3130xl Genetic Analyzer (Applied Biosystems, Foster City, Calif., USA).

### Example 2

### Construction of Thermobifida fusca Family 11 Xylanase Gene Mutants

Mutants of the Thermobifida fusca Family 11 xylanase synthetic gene were constructed by performing site-directed mutagenesis on ptfxyCBM (see Example 1) using a QUIKCHANGE® XL Site-Directed Mutagenesis Kit or a QUIKCHANGE MULTI® Site-Directed Mutagenesis Kit (Stratagene, La Jolla, Calif., USA) to generate mutants 51, 49, 340, 341, 370, 386, 473, 472, 470, 474, and 471. A summary of the oligos used for the site-directed mutagenesis and the mutants obtained are shown in Table 1.

The resulting mutant plasmid DNAs were prepared using a BIOROBOT® 9600 and sequenced using a 3130×1 Genetic Analyzer. The sequence-confirmed mutants were digested with Sac 1 and Mk/1 and purified by 1.0% agarose gel electrophoresis in TAE buffer. Fragments of 700 bp were excised from the gels and extracted using a QIAQUICK® Gel Extraction Kit. One hundred ng of each fragment were then ligated to 50 ng of SacI and Mlu I digested and purified pTH025 (as described above) using a Rapid Ligation Kit in a 10 μl reaction volume overnight at 15° C. Five μl of the ligation mixture was used to transform E. coli SURE® competent cells. Transformants were selected on LB+Amp agar medium. Plasmid DNA from E. coli transformants containing pSMO398 (L186V), pSMO396 (T74A), pSMO513 (T74S+L186V), pSMO520 (T74S+L1861), pSMO514 (T74A+L1861), pSMO512 (T74A+L186V), pSMO567 (A21S+T74S+L186V), pSMO566 (S38Y+T74S+L186V), pSMO564 (G55D+T74SL+186V), pSMO568 (T74S+N81D+L186V), or pSMO565 (S62T+T74S+L186V) was prepared using a BIOROBOT® 9600. Plasmids were sequenced using a 3130×1 Genetic Analyzer.

TABLE 1

| | Amino acid | | | |
|---|---|---|---|---|
| | changes in | | | |
| | mutagenesis | Primer | | Plasmid |
| ID | primers | name | Sequences | Name |
| 51 | L186V | 066452 | CATCAAACGTTACAGTAGGCACATCAGGAGGTG (SEQ ID NO: 7) | pSMO398 |
| | | 066453 | CACCTCCTGATGTGCCTACTGTAACGTTTGATG (SEQ ID NO: 8) | |
| 49 | T74A | 066446 | GGTAACGCTTATCTTGCACTTTACGGATGGAC (SEQ ID NO: 9) | pSMO396 |
| | | 066447 | GTCCATCCGTAAAGTGCAAGATAAGCGTTACC (SEQ ID NO: 10) | |
| 340 | T74S + | 066452 | CATCAAACGTTACAGTAGGCACATCAGGAGGTG (SEQ ID NO: 11) | pSMO513 |
| | L186V | 067726 | GGTAACGCTTATCTTTCACTTTACGGATGGAC (SEQ ID NO: 12) | |
| 370 | T74S + | 067726 | GGTAACGCTTATCTTTCACTTTACGGATGGAC (SEQ ID NO: 13) | pSMO520 |
| | L186I | 067727 | GTCCATCCGTAAAGTGAAAGATAAGCGTTACC (SEQ ID NO: 14) | |
| 341 | T74A + | 066446 | GGTAACGCTTATCTTGCACTTTACGGATGGAC (SEQ ID NO: 15) | pSMO514 |
| | L186I | 066447 | GTCCATCCGTAAAGTGCAAGATAAGCGTTACC (SEQ ID NO: 16) | |
| 386 | T74A + | 066452 | CATCAAACGTTACAGTAGGCACATCAGGAGGTG (SEQ ID NO: 17) | pSMO512 |
| | L186V | 066446 | GGTAACGCTTATCTTGCACTTTACGGATGGAC (SEQ ID NO: 18) | |
| 473 | A21S | 066452 | CATCAAACGTTACAGTAGGCACATCAGGAGGTG (SEQ ID NO: 19) | pSMO567 |
| | | 067726 | GGTAACGCTTATCTTTCACTTTACGGATGGAC (SEQ ID NO: 20) | |
| | | 068256 | ATTTTGGACAGACTCTCCTGGAACTGTATC (SEQ ID NO: 21) | |
| 472 | S38Y + | 066452 | CATCAAACGTTACAGTAGGCACATCAGGAGGTG (SEQ ID NO: 22) | pSMO566 |
| | T74S + | 067726 | GGTAACGCTTATCTTTCACTTTACGGATGGAC (SEQ ID NO: 23) | |
| | L186V | 068254 | GCAACTACTCAACGTACTGGCGCAACACAGG (SEQ ID NO: 24) | |
| 470 | G55D + | 066452 | CATCAAACGTTACAGTAGGCACATCAGGAGGTG (SEQ ID NO: 25) | pSMO564 |
| | T74S + | 067726 | GGTAACGCTTATCTTTCACTTTACGGATGGAC (SEQ ID NO: 26) | |
| | L186V | 068252 | GGCTGGGCGACAGGAGACCGTCGCACAGTTAC (SEQ ID NO: 27) | |
| 474 | T74S + | 066452 | CATCAAACGTTACAGTAGGCACATCAGGAGGTG (SEQ ID NO: 28) | pSMO568 |
| | N81D + | 067726 | GGTAACGCTTATCTTTCACTTTACGGATGGAC (SEQ ID NO: 29) | |
| | L186V | 068001 | TACGGATGGACTCGCGACCCTCTTGTTGAGTAC (SEQ ID NO: 30) | |
| 471 | S62T + | 066452 | CATCAAACGTTACAGTAGGCACATCAGGAGGTG (SEQ ID NO: 31) | pSMO565 |
| | T74S + | 067726 | GGTAACGCTTATCTTTCACTTTACGGATGGAC (SEQ ID NO: 32) | |
| | L186V | 067999 | CGCACAGTTACTTACACTGCTTCTTTCAACCCTTC (SEQ ID NO: 33) | |

### Example 3

### Expression of the Thermobifida fusca Family 11 Xylanase Variants in Bacillus subtilis

One μg of pSMO398, pSMO396, pSMO513, pSMO520, pSMO514, pSMO512, pSMO567, pSMO566, pSMO564, pSMO568, or pSMO565 (See Table 1) was linearized with Sal I. The linearized plasmids were purified using a QIAQUICK® Minelute column. Each linearized DNA was transformed into Bacillus subtilis McLp2 or McLp7.

Competent cells of B. subtilis McLp2 or McLp7 were prepared according to Anagnostopoulos and Spizizen, 1961, Journal of Bacteriology 81: 741-746. Cells were then centrifuged at 3836×g for 10 minutes. Eighteen ml of cell supernatant was added to 2 ml glycerol. The cell pellet was resuspended in the supernatant/glycerol mixture, distributed in 0.5 ml aliquots, and frozen at −70° C.

Half ml of Spizizen II medium with 2 mM EGTA was added to 0.5 ml of the frozen competent B. subtilis McLp2 or McLp7 cells. The cells were thawed in a water bath at 37° C. and divided into 18 tubes (approximately 50 μl each). One μg of each linearized mutant plasmid DNA was added to a separate aliquot of the competent cells and induced with 0.5 ml of chloramphenicol at a final concentration of 0.2 μg per ml. Linearized pSMO398, pSMO396, pSMO512, pSMO567, pSMO566, pSMO564, pSMO568, or pSMO565 was transformed to McLp2 while linearized pSMO513, pSMO520 and pSMO514 were transformed to McLp7. Transformation reactions were incubated at 37° C. for 1 hour with shaking at 250 rpm. Cells were then plated onto TBAB CM medium. The transformation of Bacillus subtilis McLp2 or McLp7 with each expression vector yielded 50-100 colonies. One colony from each transformation was streaked onto TBAB CM medium for isolation, and tested for the production of xylanase on LB 0.1% AZCL-xylan plates. Colonies positive for the production of xylanase produced blue halos on the LB 0.1% AZCL-xylan plates.

The xylanase variants were screened according to Examples 7 and 8 for improved thermostability and thermal activity.

### Example 4

### Generation of Primary Random Libraries of Thermobifida fusca Family 11 Xylanase Mutants in Bacillus subtilis McLp2

To identify regions of the Thermobifida fusca Family 11 xylanase critical for protein thermostability, the entire synthetic Thermobifida fusca Family 11 xylanase gene (see Example 1) from plasmid pTH153 was mutagenized using error-prone PCR with oligo primers designed to contain at least 30 bp of homologous sequences flanking the desired site of insertion in the Bacillus cloning vector pTH153 (Example 1). The ends were engineered in this way so that an IN-FUSION™ Advantage PCR Cloning Kit could be used to expose complementary regions on the cloning vector and DNA insert for spontaneous annealing through base pairing thus generating circular, replicating plasmids from a combination of linearized vector and PCR products.

Random mutagenesis was performed by PCR using a GENEMORPH® Random Mutagenesis II Kit (Stratagene, La Jolla, Calif., USA). Plasmid pTH153 was utilized as template DNA for PCR amplification of the Thermobifida fusca Family 11 xylanase error-prone libraries. PCR products were generated using primers Tf.xylF and Tf.xylR (Example 1). The error-prone PCR amplifications were composed of 75-100 ng of template DNA, 1× MUTAZYME® II reaction buffer (Stratagene, La Jolla, Calif., USA), 1 μl of 40 mM dNTP mix, 250 ng of each primer (Tf.xylF and Tf.xylR), and 2.5 units of MUTAZYME® II DNA polymerase (Stratagene, La Jolla, Calif., USA) in a final volume of 50 μl. The amplification reaction was performed in an EPPENDORF® MASTERCYCLER® 5333 programmed for 1 cycle at 95° C. for 2 minutes; and 30 cycles each at 95° C. for 1 minute, 55° C. for 1 minute, and 72° C. for 1 minute. After the 30 cycles, the reaction was heated for 10 minutes at 72° C. The heat block then went to a 10° C. soak cycle.

Plasmid pTH153 was gapped by digestion with Sac I and Mlu I. Fragments of 7038 bp (gapped) and 1307 bp (from the Streptococcus equisimilis hasA gene) were isolated by 0.8% agarose gel electrophoresis in TAE buffer, excised from the gel, and purified using a QIAQUICK® Minelute column.

An IN-FUSION™ Advantage PCR Cloning Kit was used to join the homologous ends of the 717 bp error-prone PCR products and plasmid pTH025, digested with Sac I and Mlu I. The PCR products contained at least 30 bp of homologous 5′ and 3′ DNA at the ends to facilitate the joining of these ends with the cloning plasmid. A total of 50 ng of each 717 bp PCR product and 100 ng of plasmid pTH025 (digested with Sac I and Mlu I) were used in a reaction containing 2 μl of 5×IN-FUSION™ reaction buffer and 1 μl of IN-FUSION™ enzyme in a final volume of 10 μl. The reactions were incubated for 15 minutes at 37° C., followed by 15 minutes at 50° C. and then placed on ice. The reaction volume was increased to 50 μl with TE buffer and 3 μl of the reaction was used to transform E. coli XL10-GOLD® Ultracompetent Cells according to the manufacturer's instructions. Transformants were selected on LB+Amp agar medium.

The resulting transformed colonies were collected in LB+Amp broth and a Plasmid Maxi Kit (QIAGEN® Inc., Valencia, Calif., USA) was used to isolate plasmid DNA from the colonies. The isolated plasmid DNA was digested with Sal I to linearize the DNA and purified using a QIAQUICK® PCR Purification Kit (QIAGEN® Inc., Valencia, Calif., USA) prior to transformation into the Bacillus subtilis McLp2 strain. Transformation of the DNA preparations into the Bacillus host was performed according to Example 3.

Two random libraries were produced, one yielding an average of 5.8 mutations per coding sequence (Library 1) and the other yielding approximately an average of 7.5 mutations per coding sequence (Library 2). It was determined that 100 ng and 75 ng of template DNA was required in the GENEMORPH® Random Mutagenesis II Kit to generate Library 1 and 2, respectively.

The xylanase variants generated from Library 1 and 2 were screened according to Examples 7 and 8 and Bacillus subtilis transformants for variants 136, 96, 101, 197, 210, 235, 291, 308, 378, 417, 425, 430, and 435 were single-colony isolated onto TBAB+Cm plates. The polynucleotide sequences for these variants were determined according to Example 9.

### Example 5

### Generation of Shuffled Mutant Thermobifida fusca GH11 Xylanase Libraries in Bacillus subtilis McLp2

The mutated DNA of Thermobifida fusca GH11 xylanase variants with improved performance in primary screens (see Examples 7 and 8) was used to generate shuffled libraries. Three libraries were created and each library was derived from the shuffling of 14, 6 or 10 improved mutants, respectively. Mutants were shuffled in vitro according to the procedure described by Stemmer, 1995, Proc. Natl. Acad. Sci. USA 91: 10747-10751. Each mutant DNA was PCR amplified from genomic DNA prepared using a REDExtract-N-Amp™ PCR ReadyMix Kit (Sigma-Aldrich, St. Louis, Mo., USA). In this extraction, a B. subtilis McLp2 mutant colony was added to 100 μl of Extraction Solution (Sigma-Aldrich, St. Louis, Mo., USA), and vortexed briefly. The extraction was incubated at 95° C. for 10 minutes and followed by the addition of 100 μl of Dilution Solution (Sigma-Aldrich, St. Louis, Mo., USA). The extraction mixture containing genomic DNA was subjected to PCR to amplify each mutant T. fusca xylanase sequence. In a 50 μl reaction each variant PCR contained 1-5 units THERMPOL II™ DNA polymerase (New England Bio Labs, Ipswich, Mass., USA), 0.2 mM of each dNTP, 50 pMol each of primer aTH153.1S and primer aTH153.1A (shown below), and 4 μl of genomic DNA prepared as described above. The reactions were incubated in an EPPENDORF® MASTERCYCLER® 5333 programmed for 1 cycle at 94° C. for 3 minutes followed by 30 cycles each at 94° C. for 30 seconds, 55° C. for 30 seconds, and 72° C. for 90 seconds (5 minute final extension).

Primer aTH153.1S:
(SEQ ID NO: 34)
5′-GCCTTACTATACCTAACATG-3′
Primer aTH153.1A:
(SEQ ID NO: 35)
5′-GAATTTAGGAGGCTTACTTGTCTGC-3′

Mutant PCR products (1.2 kb product bands) were electrophoresed on a 0.8% agarose gel in TAE buffer to quantify the DNA for DNase I digestion. Equal DNA concentrations of each mutant PCR product were combined and purified using a QIAQUICK® PCR Purification Kit, and eluted in TE buffer to deliver a final DNA concentration of 100 ng/μl mixed mutant PCR product.

The mutant PCR mix was then treated with DNase I to digest the products into small DNA fragments. In a 30 μl reaction 2 μg of mutant PCR DNA was digested with 100-500 units of DNase I (New England Bio Labs, Ipswich, Mass. USA) in 10 mM MgCl₂-0.5 M Tris pH 7.4 for 30-60 seconds at 20° C. The reaction was terminated by incubation at 95° C. for 10 minutes. Digested fragments of approximately 100 bp to 600 bp were electrophoresed on a 2% NUSIEVE™ 3:1 low melt agarose gel (FMC Bioproducts, Rockland, Me., USA) in TAE buffer, excised from the gel, and extracted using a QIAQUICK® Gel Extraction Kit.

Purified DNase I digested mutant DNA was used in a second PCR amplification to recombine and assemble 1.2 kb full-length products. The second PCR (50 μl) was composed of approximately 0.5-1.0 μg of purified DNase I digested fragments, 1× THERMPOL II™ buffer (New England Bio Labs, Ipswich, Mass., USA), 1-5 units of THERMPOL II™ DNA polymerase, and 0.2 mM of each dNTP. The reaction did not contain primer oligomers. The reactions were incubated in an EPPENDORF® MASTERCYCLER® 5333 programmed for 1 cycle at 94° C. for 1.5 minutes followed by 35 cycles each at 94° C. for 30 seconds, 65° C. for 1.5 minutes, 62° C. for 1.5 minutes, 59° C. for 1.5 minutes, 56° C. for 1.5 minutes, 53° C. for 1.5 minutes, 50° C. for 1.5 minutes, 47° C. for 1.5 minutes, 44° C. for 1.5 minutes, 41° C. for 1.5 minutes, and 72° C. for 1.5 minutes. The reactions were visualized by 1% agarose gel electrophoresis in TAE buffer for the recombined assembled 1.2 kb full-length products, excised, purified using a QIAQUICK® PCR Purification Kit, and amplified in a third PCR.

The third PCR (50 μl) was composed of 1× THERMPOL II™ buffer, 1-5 units of THERMPOL II™ DNA polymerase, 0.2 mM of each dNTP, 50 picomole each of primers Tf.xylF and Tf.xylR (Example 1), and approximately 50-100 ng of the purified recombined assembled 1.2 kb full-length products. The reactions were incubated in an EPPENDORF® MASTERCYCLER® 5333 programmed for 1 cycle at 94° C. for 3 minutes followed by 30 cycles each at 94° C. for 30 seconds, 55° C. for 30 seconds, and 72° C. for 90 seconds (5 minute final extension). The products were visualized by 1% agarose gel electrophoresis in TAE buffer for recombined amplified assembled 757 bp fragments. The fragments were excised and purified using a QIAQUICK® PCR Purification Kit.

Each final 757 bp PCR product was subcloned into plasmid pTH153 using an IN-FUSION™ Advantage PCR Cloning Kit to join the homologous ends of the 757 bp PCR product and plasmid pTH153 digested with Sac I and Mlu I. Each reaction was composed of approximately 150 ng to 200 ng of each 757 bp PCR product and 160 ng of plasmid pTH025 (digested with Sac I and Mlu I), 2 μl of 5×IN-FUSION™ reaction buffer, and 1 μl of IN-FUSION™ enzyme in a final volume of 10 μl. The reactions were incubated for 15 minutes at 37° C., followed by 15 minutes at 50° C., and then placed on ice. Each reaction volume was increased to 50 μl with TE buffer and 2-3 μl of each reaction was used to transform E. coli XL10-Gold® Ultracompetent Cells according to the manufacturer's instructions. The resulting colonies were collected in LB medium. A Plasmid Maxi Kit (QIAGEN Inc., Valencia, Calif., USA) was used to isolate plasmid DNA from the colonies. The isolated plasmids were restriction digested with Sal I to linearize the DNAs and either purified using a QIAQUICK® PCR Purification Kit or precipitated with ethanol prior to transformation into Bacillus subtilis McLp2. Transformation of each of the final DNA preparations into competent B. subtilis McLp2 was performed according to Example 3.

The T. fusca GH11 xylanase shuffled libraries were made in the B. subtilis McLp2 strain. To generate a single shuffled library, a 0.5 ml aliquot of competent B. subtilis McLp2 cells was thawed in a water bath at 37° C. and divided into 18 tubes (approximately 50 μl each). One μg of linearized shuffled plasmid DNA was added to each aliquot of competent cell mixture and induced with 0.5 ml of chloramphenicol at a final concentration of 0.2 μg per ml. The shuffled library transformation reactions were incubated at 37° C. for 1 hour with shaking at 250 rpm. Following incubation, glycerol was added to each reaction to 10% (v/v) and then frozen at −70° C. To determine the library titer (colony/μl), serial dilutions of a transformation reaction aliquot were spread onto TBAB+Cm plates and allowed to incubate for 16-20 hours at 37° C. Once the titer was determined, the shuffled library transformation reactions were thawed and plated onto screening plates and screened according to Examples 7 and 8.

Colonies of improved variants identified by the screen were single-colony isolated onto TBAB+Cm plates. The sequences for the improved variants were determined according to Example 9.

### Example 6

### Construction of Thermobifida fusca Family 11 Xylanase Variants 341, 370, 525-528, 569-583, and 529

The Thermobifida fusca Family 11 xylanase backbone for variants 370 and 341 was variant 136, which contains the substitution L1861. Variant 136 was selected as an improved performer as defined by the Thermobifida fusca Family 11 xylanase screen (Examples 7 and 8) and originated from a random library (Example 4). To generate variant 370 (T74S+L186I) from variant 136, a QUIKCHANGE® XL Site-Directed Mutagenesis Kit was used with the following forward and reverse primers:

Forward primer:
(SEQ ID NO: 36)
5′-GGTAACGCTTATCTTTCACTTTACGGATGGAC-3′
Reverse primer:
(SEQ ID NO: 37)
5′-GTCCATCCGTAAAGTGAAAGATAAGCGTTACC-3′

To generate variant 341 (T74A+L1861) from variant 136, a QUIKCHANGE® XL Site-Directed Mutagenesis Kit was used with the following forward and reverse primers:

Forward primer:
(SEQ ID NO: 38)
5′-GGTAACGCTTATCTTGCACTTTACGGATGGAC-3′
Reverse primer:
(SEQ ID NO: 39)
5′-GTCCATCCGTAAAGTGCAAGATAAGCGTTACC-3′

The resulting mutant plasmid DNAs were ligated into pTH025 and transformed into E. coli SURE® competent cells according to the procedure described in Example 2. Plasmid DNA from the E. coli transformants containing pSMO514 (T74A+L1861) and pSMO520 (T74S+L186I) were prepared using a BIOROBOT® 9600 and sequenced using a 3130xl Genetic Analyzer.

The Thermobifida fusca Family 11 xylanase backbone for variants 525, 526, 527, and 528 was variant 340, which contains the substitutions T74S and L186V. Site-directed mutagenesis was performed on variant 340 using a QUIKCHANGE® XL Site-Directed Mutagenesis Kit or a QUI KCHANGE MULTI® Site-Directed Mutagenesis Kit (Stratagene, La Jolla, Calif., USA) with the oligos shown in Table 2.

TABLE 2

| | Amino acid | | | |
|---|---|---|---|---|
| | changes | | | |
| | in | | | Cloning |
| | mutagenesis | Primer | | Plasmid |
| ID | primers | name | Sequences | Name |
| 525 | F17L + N81D + | 066452 | CATCAAACGTTACAGTAGGCACATCAGGAGGTG | pSMO583 |
| | T188A | | (SEQ ID NO: 40) | |
| | | 067726 | GGTAACGCTTATCTTTCACTTTACGGATGGAC | |
| | | | (SEQ ID NO: 41) | |
| | | 068252 | GGCTGGGCGACAGGAGACCGTCGCACAGTTAC | |
| | | | (SEQ ID NO: 42) | |
| 526 | V2I + R57H | 068003 | CGCTTCTGCTGCAATCACTTCTAACGAGACAG | pSMO584 |
| | | | (SEQ ID NO: 43) | |
| | | 068250 | GCGACAGGAGGTCGTCATACAGTTACTTACTC | |
| | | | (SEQ ID NO: 44) | |
| 527 | R57H | 068250 | GCGACAGGAGGTCGTCATACAGTTACTTACTC | pSMO585 |
| | | | (SEQ ID NO: 45) | |
| | | 068251 | GAGTAAGTAACTGTATGACGACCTCCTGTCGC | |
| | | | (SEQ ID NO: 46) | |
| 528 | N41D | 068506 | CTCAACGTCTTGGCGCGACACAGGAAACTTCG | pSMO586 |
| | | | (SEQ ID NO: 47) | |
| | | 068507 | CGAAGTTTCCTGTGTCGCGCCAAGACGTTGAG | |
| | | | (SEQ ID NO: 48) | |

The resulting mutant plasmid DNAs were ligated into pTH025 and transformed into E. coli SURE® competent cells according to the procedure described in Example 2. Plasmid DNA from the E. coli transformants containing pSMO583 (F17L+N81D+T188A+T74S+L186V); pSMO584 (V2I+R57H+T74S+L186V); pSMO585 (R57H+ T74S+L186V); and pSMO586 (N41D+T74S+L186V) were prepared using a BIOROBOT® 9600 and sequenced using a 3130×1 Genetic Analyzer.

The Thermobifida fusca Family 11 xylanase backbone for variants 569-577 was variant 473, which contains the substitutions A21S, T74S, and L186V. Site-directed mutagenesis was performed on variant 473 using a QUIKCHANGE MULTI® Site-Directed Mutagenesis Kit with the following oligos:

N81D:
(SEQ ID NO: 49)
5′-TACGGATGGACTCGCGACCCTCTTGTTGAGTAC-3′
S38Y:
(SEQ ID NO: 50)
5′-GCAACTACTCAACGTACTGGCGCAACACAGG-3′
S62T:
(SEQ ID NO: 51)
5′-CGCACAGTTACTTACACTGCTTCTTTCAACCCTTC-3′

The resulting plasmid DNAs were prepared using a BIOROBOT® 9600 and sequenced using a 3130xl Genetic Analyzer. Plasmid DNA from the E. coli transformants containing pSMO611 (A21S+T74S+N81D+L186V; ID569); pSMO611 (A21S+S38Y+T74S+L186V; ID570); pSMO612 (A21S+S38Y+T74S+N81D+L186V; ID572); pSMO614 (A21S+S62Y+T74S+N81D+L186V; ID573); pSMO615 (A21S+S38Y+S62T+T74S+L186V; ID574); pSMO616 (A21S+S38Y+S62T+T74S+N81D+L186V; ID575); and pSMO617 (A21S+S62T+T74S+L186V; ID577) were prepared using a BIOROBOT® 9600 and sequenced using a 3130×1 Genetic Analyzer.

In a separate reaction, site-directed mutagenesis was performed on mutant 473 using a QUIKCHANGE MULTI® Site-Directed Mutagenesis Kit with oligos N81D and S38Y and the following oligo:

G55D:
(SEQ ID NO: 52)
5′-GGCTGGGCGACAGGAGACCGTCGCACAGTTAC-3′

The resulting mutant plasmid DNAs were ligated into pTH025 and transformed into E. coli SURE® competent cells according to the procedure described in Example 2. Plasmid DNA from the E. coli transformants containing pSMO613 (A21S+G55D+T74S+L186V; ID571) and pSMO618 (A21S+S38Y+G55D+T74S+N81D+L186V; ID576) were prepared using a BIOROBOT® 9600 and sequenced using a 3130×1 Genetic Analyzer.

To generate variants 578-583, site-directed mutagenesis was performed using a QUIKCHANGE® XL Site-Directed Mutagenesis Kit on various mutant templates, described above originally generated from mutant 473. Oligomers and template ID mutants are described in Table 3.

TABLE 3

| | Amino acid | | | |
|---|---|---|---|---|
| | changes | | | |
| | in | | | |
| Template | mutagenesis | Primer | | Resultant |
| mutant ID | primers | name | Sequences | mutant ID |
| 569 | G55D | 068252 | GGCTGGGCGACAGGAGACCGTCGCACAGTTAC | 578 |
| | | | (SEQ ID NO: 53) | |
| | | 068253 | GTAACTGTGCGACGGTCTCCTGTCGCCCAGCC | |
| | | | (SEQ ID NO: 54) | |
| 570 | G55D | 068252 | GGCTGGGCGACAGGAGACCGTCGCACAGTTAC | 579 |
| | | | (SEQ ID NO: 55) | |
| | | 068253 | GTAACTGTGCGACGGTCTCCTGTCGCCCAGCC | |
| | | | (SEQ ID NO: 56) | |
| 571 | S62T | 067999 | CGCACAGTTACTTACACTGCTTCTTTCAACCCTTC | 580 |
| | | | (SEQ ID NO: 57) | |
| | | 068000 | GAAGGGTTGAAAGAAGCAGTGTAAGTAACTGTGCG | |
| | | | (SEQ ID NO: 58) | |
| 574 | G55D | 068252 | GGCTGGGCGACAGGAGACCGTCGCACAGTTAC | 581 |
| | | | (SEQ ID NO: 59) | |
| | | 068253 | GTAACTGTGCGACGGTCTCCTGTCGCCCAGCC | |
| | | | (SEQ ID NO: 60) | |
| 573 | G55D | 068252 | GGCTGGGCGACAGGAGACCGTCGCACAGTTAC | 582 |
| | | | (SEQ ID NO: 61) | |
| | | 068253 | GTAACTGTGCGACGGTCTCCTGTCGCCCAGCC | |
| | | | (SEQ ID NO: 62) | |
| 576 | S62T | 067999 | CGCACAGTTACTTACACTGCTTCTTTCAACCCTTC | 583 |
| | | | (SEQ ID NO: 63) | |
| | | 068000 | GAAGGGTTGAAAGAAGCAGTGTAAGTAACTGTGCG | |
| | | | (SEQ ID NO: 64) | |

The resulting mutant plasmid DNAs were ligated into pTH025 and transformed into E. coli SURE® competent cells according to the procedure described in Example 2. Plasmid DNA from the E. coli transformants containing pSMO611 (A21S+T74S+N81D+L186V; ID 569); pSMO612 (A21S+S38Y+T74S+L186V; ID 570); pSMO613 (A21S+G55D+T74S+L186V; ID 571); pSMO614 (A21S+S38Y+T74S+N81D+L186V; ID 572); pSMO615 (A21S+S62Y+T74S+N81D+L186V; ID 573); pSMO616 (A21S+S38Y+S62T+T74S+L186V; ID 574); pSMO617 (A21S+S38Y+S62T+T74S+N81D+L186V; ID 575); pSMO618 (A21S+S38Y+G55D+T74S+N81D+L186V; ID 576); pSMO619 (A21S+S62T+T74S+L186V; ID 577); pSMO620 (A21S+G55D+T74S+N81D+L186V; ID 578); pSMO621 (A21S+S38Y+G55D+T74S+L186V; ID 579); pSMO622 (A21S+G55D+S62T+T74S+L186V; ID 580); pSMO623 (A21S+S38Y+G55D+S62T+T74S+L186V; ID 581); pSMO624 (A21S+G55D+S62T+T74S+N81D+L186V; ID 582); and pSMO625 (A21S+S38Y+G55D+S62T+T74S+N81D+L186V; ID 583) were prepared using a BIOROBOT® 9600 and sequenced using a 3130×1 Genetic Analyzer.

Bacillus subtilis McLp2 was transformed with each of the plasmids above and grown to produce the xylanase variants according to Example 3. In addition, pSMO513 (T74S, L186V; with previous McLp7 variant ID 340), described in Example 3, was transformed into McLp2 resulting in variant 529 to ensure similar expression level as other McLp2 derived variants. The Thermobifida fusca Family 11 xylanase variants above were screened according to Examples 7 and 8.

### Example 7

### Screening of Thermobifida fusca Family 11 Xylanase Libraries

Primary Thermobifida fusca Family 11 xylanase mutant libraries in Bacillus subtilis McLp2 were spread on LB+Cm agar medium with 0.1% AZCL-Arabinoxylan wheat (Megazyme Wicklow, Ireland) in Genetix QTrays (22×22 cm Petri dishes, Genetics Ltd., Hampshire, United Kingdom) and incubated for 1 day at 37° C. Bacillus subtilis colonies producing xylanase yield a blue halo around the colonies. Using a QPix System (Genetix Ltd., Hampshire, United Kingdom), active colonies were picked into 96-well plates containing 1/3 diluted MY25 medium. Plates were incubated for 4 days at 37° C. with agitation at 250 rpm. After the incubation, the plates were diluted with 0.01% TWEEN® 20 in deionized water using a BIOMEK® FX Laboratory Automation Workstation (Beckman Coulter, Fullerton, Calif., USA). Using an ORCA robot (Beckman Coulter, Fullerton, Calif., USA), the diluted plates were transported to a BIOMEK® FX and 10 μl of the diluted samples were removed from the plate and aliquoted into two 96-well polycarbonate v-bottom plates. Forty μl of 0.01% TWEEN® 20-125 mM sodium borate pH 8.8 were added to the assay plates. The assay plates were transferred to a temperature-controlled incubator, where one plate was incubated at room temperature for 15 minutes, and another was incubated at a pre-determined temperature, between 80° C. and 90° C., for 15 minutes. After this incubation, the assay plates were transferred to the BIOMEK® FX and 30 μl of 0.01% TWEEN® 20-0.5% w/v AZCL-xylan oat (Megazyme Wicklow, Ireland) substrate and 80 μl of 0.01% TWEEN® 20-125 mM sodium borate pH 8.8 were added. The assay plates were transferred back to a temperature-controlled incubator, where both plates were incubated at 50° C. for 15 minutes. After the incubation, the assay plates were transferred back to the BIOMEK® FX for mixing and settling for 30 minutes. After 30 minutes, 60 μl of supernatants were removed from the plates and transferred to 384-well polypropylene flat bottom plates. The 384-well plates were transferred to a DTX microplate reader (Beckman Coulter, Fullerton, Calif., USA) and the absorbance was measured at 595 nm.

The ratio of the absorbance from the plates treated at high temperature (“heat-treated activity”) was compared to absorbance from the same samples incubated at room temperature (“non-heat-treated activity”), using MICROSOFT® EXCEL® (Microsoft Corporation, Redmond, Wash., USA) to determine the relative thermostability ratio for each variant. Based on the thermostability ratios, screening of libraries constructed in the previous Examples generated the variants listed in Table 4. To measure the improvement in thermostability relative to the parent xylanase, the thermostability ratio of each variant was normalized to the thermostability ratio of the parent xylanase, which is marked “Fold Improvement” in Table 4. The fold improvement in thermostability for the Thermobifida fusca Family 11 xylanase variants ranged from 1.1 to 2.28 (Table 4). Table 4 demonstrates the degree of improvement in thermostability for the Thermobifida fusca Family 11 xylanase variants. For variants obtained in the primary screen, improvements in thermostability ranged from 1.1-fold to 1.6-fold relative to the parent xylanase. For variants obtained from site-directed mutagenesis (SDM), the improvement in thermostability observed was 1.1-fold to 2.28-fold relative to the parent xylanase at 80° C. Table 5 lists the improved variants that were tested at 85° C. The fold improvement in thermostability for these Thermobifida fusca Family 11 xylanase variants ranged from 1.2 to 3.9 at 85° C. (Table 5).

TABLE 4
Variants with improved thermostability at 80° C.

| | | Fold | |
|---|---|---|---|
| | | Improve- | |
| ID | Variants | ment | Type |
| Parent | — | 1 | — |
| 51 | L186V | 1.43 | SDM |
| 136 | L186I | 1.28 | Random library |
| 49 | T74A | 1.1 | SDM |
| 96 | T74S | 1.21 | Random library |
| 340 | T74S + L186V | 1.77 | SDM |
| 370 | T74S + L186I | 1.58 | SDM |
| 341 | T74A + L186I | 1.5 | SDM |
| 386 | T74A + L186V | 1.55 | SDM |
| 473 | A21S + T74S + L186V | 2.28 | SDM |
| 472 | S38Y + T74S + L186V | 2.17 | SDM |
| 470 | G55D + T74S + L186V | 2.16 | SDM |
| 474 | T74S + N81D + L186V | 2.11 | SDM |
| 471 | S62T + T74S + L186V | 2.05 | SDM |
| 462 | S38Y + L186V | 1.59 | Shuffled library |
| 461 | T74A + N81D + L186V | 1.64 | Shuffled library |
| 425 | E28V + R56H + N183D | 1.38 | Random library |
| 197 | F17L + N81D + T188A | 1.32 | Random library |
| 435 | S38F + G192D | 1.30 | Random library |
| 417 | R56P + T60S | 1.27 | Random library |
| 210 | V2I + R57H | 1.27 | Random library |
| 430 | A21S | 1.26 | Random library |
| 308 | F17L + N81D | 1.22 | Random library |
| 291 | N81D | 1.24 | Random library |
| 378 | S38Y + T104S | 1.22 | Random library |
| 235 | F17L + M161L | 1.20 | Random library |
| 101 | G55D | 1.20 | Random library |

TABLE 5
Variants with improved thermostability at 85° C.

| | | Fold | |
|---|---|---|---|
| | | Improve- | |
| ID | Variants | ment | Type |
| Par- | — | 1.0 | — |
| ent | | | |
| 136 | L186I | 1.2 | Random Library |
| 370 | T74S + L186I | 1.2 | SDM |
| 470 | G55D + T74S + L186V | 2.5 | SDM |
| 471 | S62T + T74S + L186V | 2.2 | SDM |
| 472 | S38Y + T74S + L186V | 2.4 | SDM |
| 473 | A21S + T74S + L186V | 2.7 | SDM |
| 474 | T74S + N81D + L186V | 2.2 | SDM |
| 486 | V2I + T74S + H159R + L186V | 2.2 | Shuffled library |
| 493 | V2I + F17L + T74S + L186I | 2.2 | Shuffled library |
| 510 | V2I + S62T + T74S + L186V | 2.8 | Shuffled library |
| 516 | V2I + T74S + L186V | 2.5 | Shuffled library |
| 518 | V2I + T74S + N81D + L186V | 2.6 | Shuffled library |
| 525 | F17L + T74S + N81D + L186V + | 2.3 | SDM |
| | T188A | | |
| 526 | V2I + R57H + T74S + L186V | 2.5 | SDM |
| 527 | R57H + T74S + L186V | 2.0 | SDM |
| 528 | N41D + T74S + L186V | 2.1 | SDM |
| 529 | T74S + L186V | 1.9 | SDM |
| 564 | V2I + E28V + S38Y + S62T + | 3.8 | Shuffled library |
| | T74S + T111I + L186V | | |
| 566 | E28V + S38Y + T74S + N121Y + | 2.5 | Shuffled library |
| | N151D + L186V | | |
| 567 | A21S + S38Y + G55D + T74S + | 3.3 | Shuffled library |
| | L186V | | |
| 569 | A21S + T74S + N81D + L186V | 2.9 | SDM |
| 570 | A21S + S38Y + T74S + L186V | 3.3 | SDM |
| 571 | A21S + G55D + T74S + L186V | 3.2 | SDM |
| 572 | A21S + S38Y + T74S + N81D + | 3.5 | SDM |
| | L186V | | |
| 573 | A21S + S62Y + T74S + N81D + | 3.3 | SDM |
| | L186V | | |
| 574 | A21S + S38Y + S62T + T74S + | 3.5 | SDM |
| | L186V | | |
| 575 | A21S + S38Y + S62T + T74S + | 3.7 | SDM |
| | N81D + L186V | | |
| 576 | A21S + S38Y + G55D + T74S + | 3.6 | SDM |
| | N81D + L186V | | |
| 577 | A21S + S62T + T74S + L186V | 3.0 | SDM |
| 578 | A21S + G55D + T74S + N81D + | 3.3 | SDM |
| | L186V | | |
| 579 | A21S + S38Y + G55D + T74S + | 3.6 | SDM |
| | L186V | | |
| 580 | A21S + G55D + S62T + T74S + | 3.4 | SDM |
| | L186V | | |
| 581 | A21S + S38Y + G55D + S62T + | 3.9 | SDM |
| | T74S + L186V | | |
| 582 | A21S + G55D + S62T + T74S + | 3.6 | SDM |
| | N81D + L186V | | |
| 583 | A21S + S38Y + G55D + S62T + | 3.9 | SDM |
| | T74S + N81D + L186V | | |

### Example 8

### Thermal Activity of Thermobifida fusca Family 11 Xylanase Variants

Improved variants from the thermostability screen in Example 7 were re-grown in a 24 well plate containing 1/3 diluted MY25 medium. Plates were incubated for 4 days at 37° C. at 250 rpm. After the incubation, the plates were diluted with 0.01% TWEEN® 20 deionized-water using a BIOMEK® FX workstation. Using the BIOMEK® FX workstation, 10 μl of the diluted samples were removed from the plates and aliquoted into two 96-well polycarbonate v-bottom plates. Fifty μl of 125 mM sodium borate pH 8.8 in 0.01% TWEEN® 20 and 40 μl of 0.5% w/v AZCL-xylan oat substrate in 0.01% TWEEN® 20 were added to the assay plates. The assay plates were transferred to a temperature-controlled incubator, where one plate was incubated at 27° C. for 15 minutes, and another was incubated at a pre-determined temperature, between 80° C. and 90° C., for 15 minutes. After the incubation, the assay plates were transferred back to the BIOMEK® FX for mixing and settling for 30 minutes. After 30 minutes, 60 μl of each supernatant were removed from the plates and transferred to 384-well polypropylene flat bottom plates. The 384-well plates were transferred to a DTX reader (Beckman Coulter, Fullerton, Calif., USA) and read at 595 nm absorbance. The assay steps above were repeated for 60 minutes instead of 15 minutes.

The absorbance from the plate treated at 80° C. for 60 minutes was subtracted from the absorbance from the plate treated at 80° C. for 15 minutes, denoted as “80° C. activity (60-15 minutes)”. The absorbance activity from the plate treated at 27° C. for 60 minutes was subtracted from the absorbance activity from the plate treated at 27° C. for 15 minutes, denoted as “27° C. activity (60-15 minutes)”. The ratio of 80° C. activity (60-15 minutes) was compared to 27° C. activity (60-15 minutes) was compared with the same samples, using MICROSOFT® EXCEL® to determine the relative thermal activity ratio for each variant. To measure the improvement in thermal activity relative to the parent xylanase, the thermal activity ratio of each variant was normalized to the thermal activity ratio of the parent xylanase, which is designated “Fold Improvement” in Table 6. Table 6 demonstrates the degree of improvement in thermal activity for the Thermobifida fusca Family 11 xylanase variants relative to the original parent xylanase (1.0). These variants had 7.7-fold to 164.8-fold improvements in thermal activity relative to the parent xylanase.

TABLE 6
Variants with improved thermal activity

| | | Fold | |
|---|---|---|---|
| | | Improve- | |
| ID | Variants | ment | Type |
| Par- | — | 1.0 | — |
| ent | | | |
| 136 | L186I | 7.7 | Random Library |
| 370 | T74S + L186I | 43.2 | SDM |
| 470 | G55D + T74S + L186V | 57.9 | SDM |
| 471 | S62T + T74S + L186V | 76.4 | SDM |
| 472 | S38Y + T74S + L186V | 86.8 | SDM |
| 473 | A21S + T74S + L186V | 82.6 | SDM |
| 474 | T74S + N81D + L186V | 108.7 | SDM |
| 486 | V2I + T74S + H159R + L186V | 59.1 | Shuffled library |
| 493 | V2I + F17L + T74S + L186I | 81.9 | Shuffled library |
| 510 | V2I + S62T + T74S + L186V | 127.3 | Shuffled library |
| 516 | V2I + T74S + L186V | 77.2 | Shuffled library |
| 518 | V2I + T74S + N81D + L186V | 12.9 | Shuffled library |
| 525 | F17L + T74S + N81D + L186V + | 49.2 | SDM |
| | T188A | | |
| 526 | V2I + R57H + T74S + L186V | 53.3 | SDM |
| 527 | R57H + T74S + L186V | 51.7 | SDM |
| 528 | N41D + T74S + L186V | 17.7 | SDM |
| 529 | T74S + L186V | 58.6 | SDM |
| 564 | V2I + E28V + S38Y + S62T + | 153.9 | Shuffled library |
| | T74S + T111I + L186V | | |
| 566 | E28V + S38Y + T74S + N121Y + | 164.8 | Shuffled library |
| | N151D + L186V | | |
| 567 | A21S + S38Y + G55D + T74S + | 74.3 | Shuffled library |
| | L186V | | |
| 569 | A21S + T74S + N81D + L186V | 53.7 | SDM |
| 570 | A21S + S38Y + T74S + L186V | 72.6 | SDM |
| 571 | A21S + G55D + T74S + L186V | 131.4 | SDM |
| 572 | A21S + S38Y + T74S + N81D + | 82.4 | SDM |
| | L186V | | |
| 573 | A21S + S62Y + T74S + N81D + | 72.6 | SDM |
| | L186V | | |
| 574 | A21S + S38Y + S62T + T74S + | 109.6 | SDM |
| | L186V | | |
| 575 | A21S + S38Y + S62T + T74S + | 136.3 | SDM |
| | N81D + L186V | | |
| 576 | A21S + S38Y + G55D + T74S + | 116.0 | SDM |
| | N81D + L186V | | |
| 577 | A21S + S62T + T74S + L186V | 116.0 | SDM |
| 578 | A21S + G55D + T74S + N81D + | 110.1 | SDM |
| | L186V | | |
| 579 | A21S + S38Y + G55D + T74S + | 114.9 | SDM |
| | L186V | | |
| 580 | A21S + G55D + S62T + T74S + | 126.1 | SDM |
| | L186V | | |
| 581 | A21S + S38Y + G55D + S62T + | 116.7 | SDM |
| | T74S + L186V | | |
| 582 | A21S + G55D + S62T + T74S + | 106.5 | SDM |
| | N81D + L186V | | |
| 583 | A21S + S38Y + G55D + S62T + | 96.9 | SDM |
| | T74S + N81D + L186V | | |

### Example 9

### Determination of Xylanase Mutation Sequences by DNA Sequencing

To determine the sequences of the Thermobifida fusca GH11 xylanase mutants derived from the libraries of the previous Examples, genomic PCR fragments containing the Thermobifida fusca xylanase mutant genes were isolated. Each Bacillus subtilis transformant containing a xylanase mutant gene was streaked onto TBAB+Cm plates and incubated for 1 day at 37° C. Extraction of DNA from the Bacillus subtilis colonies was performed using a REDExtract-N-Amp™ Plant PCR Kit (Sigma-Aldrich, St. Louis, Mo., USA) with a slight modification. One Bacillus subtilis colony was added to 100 μl of Extraction Solution, and tubes were closed and vortexed briefly. The reaction was incubated at 95° C. for 10 minutes. Then 100 μl of Dilution Solution was added and vortexed to mix. The diluted extract was subjected to PCR amplification immediately as described below. The rest of the diluted extract was stored at 4° C.

Primers Tf.xylF and Tf.xylR (Example 1) were used to PCR amplify polynucleotides encoding the Thermobifida fusca GH11 xylanase mutant sequences from the genomic DNA extracts. A total of 0.4 μM of each primer Tf.xylF and Tf.xylR were used in PCR reactions containing 4 μl of each DNA extract and 10 μl of REDExtract-N-Amp™ PCR ReadyMix (Sigma-Aldrich, St. Louis, Mo., USA) in a final volume of 20 μl. The amplification reactions were performed in a EPPENDORF® MASTERCYCLER® ep gradient S thermocycler (Eppendorf, Hamburg, Germany) programmed for 1 cycle at 94° C. for 3 minutes; and 35 cycles each at 94° C. for 1 minute, 57° C. for 1 minute, and 72° C. for 1 minutes. After 35 cycles, the reactions were heated for 10 minutes at 72° C. The heat block then went to a 4° C. soak cycle.

The reaction products were visualized by loading 5 μl of the PCR product onto 1.0% agarose gel in 89 mM Tris base-89 mM boric acid-2 mM disodium EDTA (TBE) buffer where a 0.6 kb product band was observed for each mutant. The remainder of the PCR products (15 μl) was then purified using a QIAQUICK® PCR Purification Kit according to the manufacturer's instructions.

DNA sequencing of the PCR products was performed using a 3130xl Genetic Analyzer using dye terminator chemistry (Giesecke et al., 1992, Journal of Virol. Methods 38: 47-60). The entire coding region for each Thermobifida fusca GH11 xylanase mutant was sequenced using 10 ng of plasmid DNA and 1.6 μmol of primers Tf.xylF and Tf.xylR.

Sequence trace files were assembled, and sequence mutations were determined using a program that performs automatic assembly of sequence reads of the variants followed by comparison to the parent sequence to determine amino acid residue changes.

### Example 10

### Production of Thermobifida fusca GH11 Xylanase Variants from Bacillus subtilis McLp2

Each Bacillus subtilis McLp2 strain expressing a Thermobifida fusca Family 11 xylanase variant identified from screening was spread onto TBAB+Cm agar plates for single colony isolation and incubated for 1 day at 37° C. One colony per Bacillus strain for each variant was used to inoculate a 1 L Erlenmeyer shake flask containing 100 ml of DIFCO™ Lactobacilli MRS medium. Shake flasks were incubated for 3 days at 37° C. with agitation at 250 rpm. After the incubation, the broths were centrifuged at 5524×g for 20 minutes and the supernatants were collected for purification.

### Example 11

### Purification of Thermobifida fusca GH11 Xylanase Variants from Bacillus subtilis McLp2

The harvested broths obtained in Example 10 were each sterile filtered using a 0.22 μm polyethersulfone membrane (Millipore, Bedford, Mass., USA). The filtered broths were each desalted with 20 mM Tris-HCl pH 8.5 using an approximately 500 ml SEPHADEX™ G25 Fine column (GE Healthcare, Piscataway, N.J., USA). The desalted materials were each then submitted to a 30 ml Q-SEPHAROSE™ High Performance (GE Healthcare, Piscataway, N.J., USA) column and each xylanase variant was collected in the flow through material. The pH of the flow through material was adjusted to 5.0 using 10% acetic acid before application to a 20 ml MONO S™ column (GE Healthcare, Piscataway, N.J.). Bound proteins were eluted with a salt gradient (4 column volumes) 0 M NaCl to 100 mM NaCl in 50 mM sodium acetate pH 5.0. Fractions were examined on 8-16% CRITERION™ Stain Free SDS-PAGE gels (Bio-Rad, Hercules, Calif., USA). Fractions containing pure Thermobifida fusca GH11 xylanase variant were pooled and protein concentrations were determined by measuring the absorbance at 280/260 nm and using the calculated extinction coefficient of 2.9 (mg/ml)⁻¹*cm⁻¹.

### Example 12

### Determination of Melting Temperature of Thermobifida fusca Family 11 Xylanase Variants

The thermostability of several xylanase variants was determined by Differential Scanning calorimetry (DSC) using a VP-DSC Differential Scanning calorimeter (MicroCal Inc., Piscataway, N.J., USA). The thermal denaturation temperature, Td (° C.), was taken as the top of denaturation peak (major endothermic peak) in thermograms (Cp vs. T) obtained after heating variant enzyme solutions in 50 mM glycine pH 9.0 at a constant programmed heating rate.

Sample and reference solutions were carefully degassed immediately prior to loading of samples into the calorimeter (reference: buffer without enzyme). Sample and reference solutions (approx. 0.5 ml) were thermally pre-equilibrated for 20 minutes at 10° C. and the DSC scan was performed from 10° C. to 100° C. at a scan rate of 90 K/hr. Denaturation temperatures were determined at an accuracy of approximately +/−1° C.

The results of the thermostability determination of the xylanase variants are shown in Table 7.

TABLE 7

| Variants | T_{d} (° C.) |
|---|---|
| Parent | 85 |
| 49 | 86 |
| 51 | 90 |
| 91 | 88 |
| 94 | 87 |
| 96 | 88 |
| 101 | 86 |
| 106 | 88 |
| 110 | 84 |
| 131 | 85 |
| 136 | 91 |
| 225 | 85 |
| 235 | 87 |
| 254 | 89 |
| 265 | 90 |
| 340 | 92 |
| 341 | 90 |
| 370 | 91 |
| 473 | 94 |
| 564 | 96 |
| 566 | 94 |
| 575 | 96 |

### Example 13

### Determination of Bleach Boosting Performance of Thermobifida fusca Family 11 xylanase variants

The bleach boosting performance of T. fusca xylanase variant 136 (L1861), variant 370 (T74S+L1861), variant 564 (V2I+E28V+S38Y+S62T+T74S+T111I+L186V), or variant 566 (E28V+S38Y+T74S+N121Y+N151D+L186V) was evaluated in a Totally Chlorine Free (TCF) bleaching sequence and compared with the wild-type T. fusca xylanase.

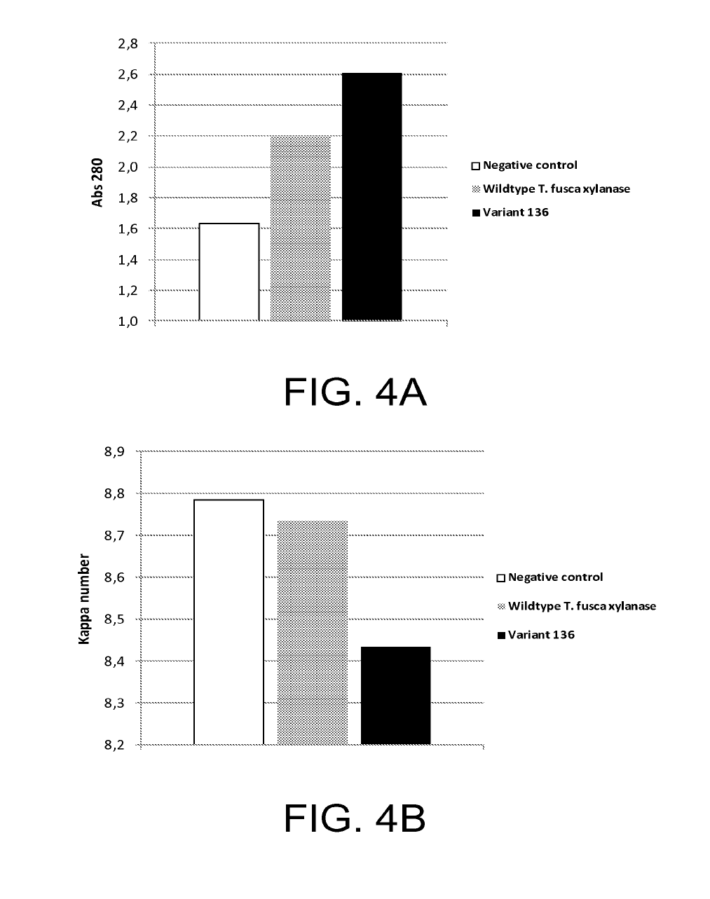

An XQP-sequence (X designates xylanase stage, Q chelation stage, and P hydrogen peroxide stage) was used under the conditions mentioned in Table 8 to analyze the pre-bleaching effect of the different xylanases. Washed unbleached eucalyptus kraft pulp of 10% pulp consistency in Britton & Robinsson buffer was treated with T. fusca xylanase variant 136, variant 370, variant 564, or variant 566, or wild-type T. fusca xylanase at 4 mg/kg of dry pulp in Stomacher® bags (BA 6040; Seward Ltd, West Sussex, UK). The amount of pulp was 8 g dry pulp per bag. The xylanase treatments were performed at pH 9.5 and 70° C. or 80° C. for 2 hours. The reference pulp (negative control) was treated in the same way but without xylanase addition. The high lignin content extracted in the filtrates (measured by A₂₈₀) and low lignin content in the XQP-bleached pulp (measured by kappa number) reflect bleach boosting effect. After the xylanase treatment, samples of the filtrates were collected for analysis. The water in the pulp was removed by filtration through a Büchner funnel and the filtrates were analyzed spectrophotometrically for release of chromophores at 280 nm (released lignin gives an absorbance at 280 nm). The Q and P stage were also performed in Stomacher® bags and the conditions for the Q and P stage are summarized in Table 8. The pulp was filtered and washed after the Q stage. After the bleaching, the hydrogen peroxide was removed from the pulp samples by filtration using a Büchner funnel. The samples were then washed thoroughly. After the washing, the pulp samples were re-suspended in water to a consistency of 0.4%. The pH of the pulp was adjusted with H₂SO₄ (to pH 2). After 20 minutes the pulp was drained using a Büchner funnel and washed with deionized water. The pulp pad was air-dried overnight. The kappa number was determined on approximately 0.5 to 1 g pulp samples using a scaled-down version of the Technical Association of the Pulp and Paper Industry (TAPPI) standard method T236. KAPPA number is defined as the number of milliliters of 20 mM potassium permanaganate solution that is consumed by 1 g of moisture-free pulp under specified conditions (results corrected for 50% consumption of the permanaganate added). All experiments were performed in duplicate and the mean values are presented in FIGS. 4-7.

TABLE 8
TCF bleaching conditions for the evaluation
of the bleach boosting effects

| STAGE | X | Q | P |
|---|---|---|---|
| Amount of treated pulp (g) | 8 | 8 | 8 |
| Consistency (%) | 10 | 10 | 10 |
| Retention time (minutes) | 120 | 60 | 150 |
| Temperature (° C.) | 70 or 80 | 70 | 90 |
| pH | 9.5 | 6-7 | 11 |
| Enzyme dosage (mg/kg dry pulp) | 4 | — | — |
| EDTA (% of dry matter) | — | 0.2 | — |
| MgSO₄ (% of dry matter) | — | — | 0.1 |
| NaOH (% of dry matter) | — | — | 1.33 |
| H₂O₂ (% of dry matter) | — | — | 1.5 |

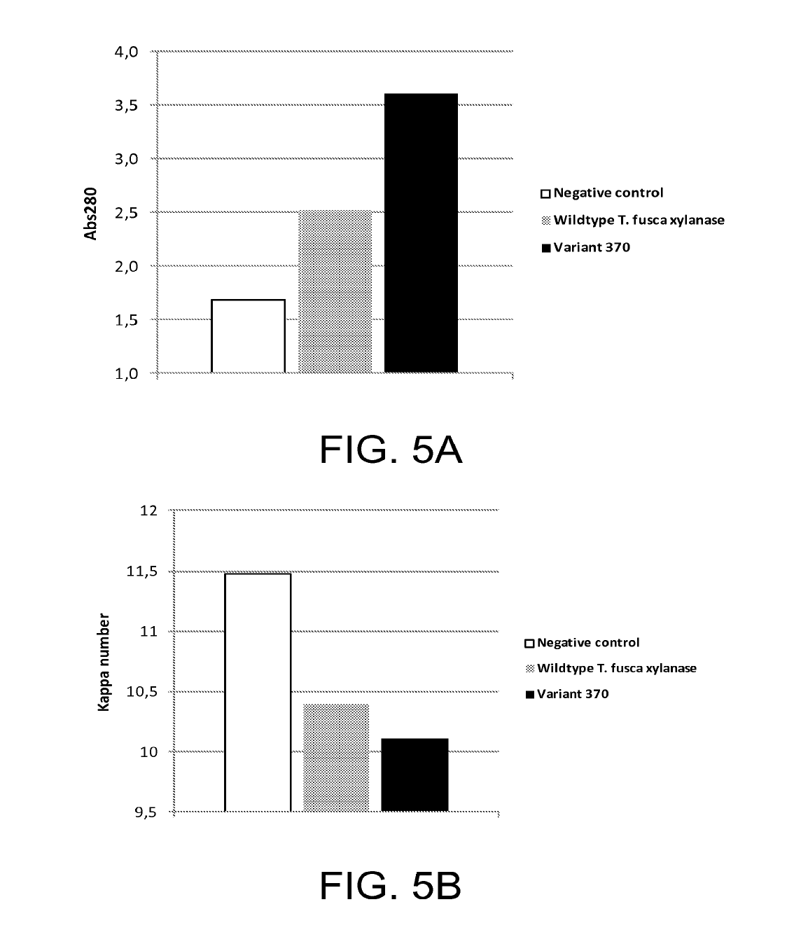

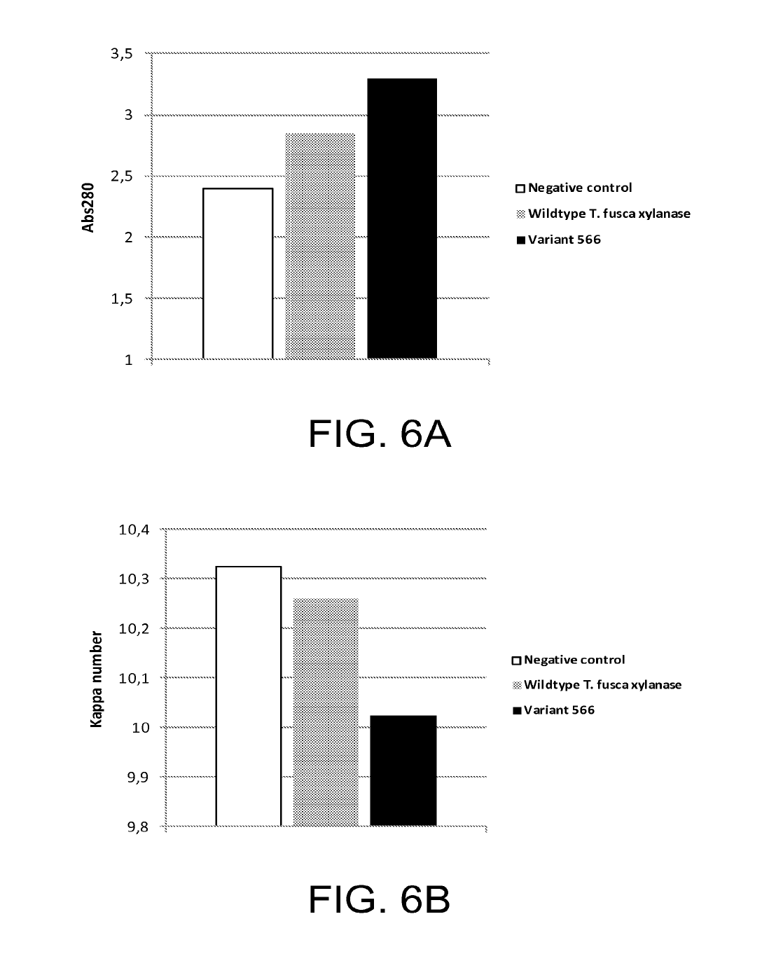

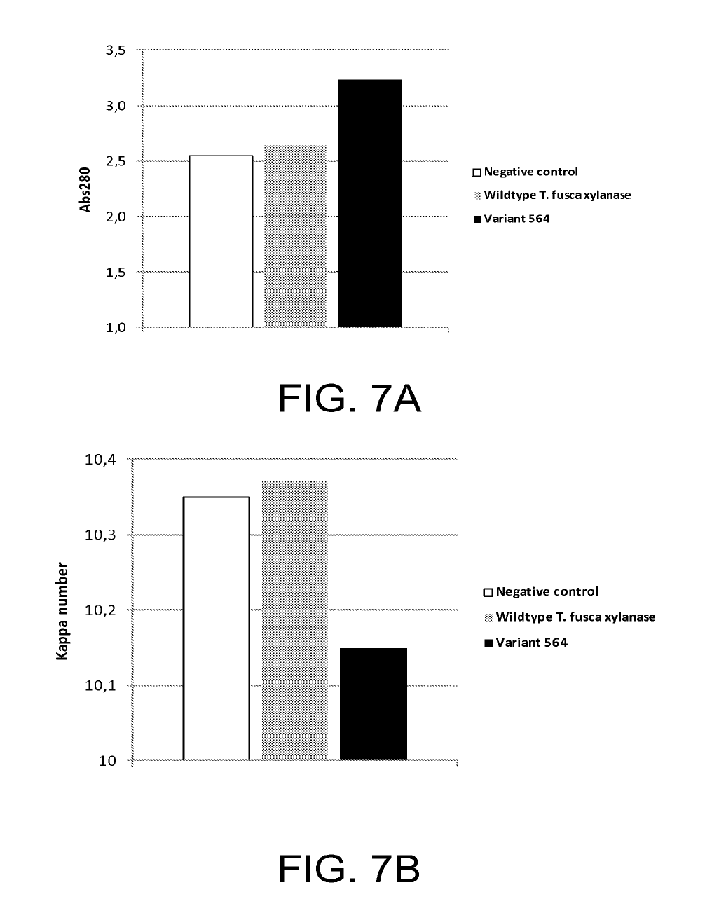

The results from the bleaching experiments are shown in FIGS. 4-7. Spectrophotometric as well as kappa number measurements showed that the T. fusca xylanase variants 136 (L1861) and 370 (T74S+L1861) yielded higher kappa number reduction and release of 280 nm absorbing material than the wild-type T. fusca xylanase at 70° C. and pH 9.5 (FIGS. 4-5). At 80° C. and pH 9.5, T. fusca xylanase variants 564 (V2I+E28V+S38Y+S62T+T74S+T111I+L186V) and 566 (E28V+S38Y+T74S+N121Y+N151D+L186V) liberated more chromophoric material and lowered the kappa number more than the wild-type T. fusca xylanase (FIGS. 6-7). The results indicated that the substitution L186I in T. fusca xylanase variant 136, substitutions T74S+L1861 in variant 370, substitutions V2I+E28V+S38Y+S62T+T74S+T111I+L186V in variant 564, and substitutions E28V+S38Y+T74S+N121Y+N151D+L186V in variant 566 improved the bleach boosting performance at high temperatures and pH 9.5.

The present invention is described by the following numbered paragraphs:

[1] An isolated variant of a parent xylanase, comprising a substitution at one or more positions corresponding to positions 2, 17, 21, 28, 38, 41, 55, 56, 57, 60, 62, 74, 81, 104, 111, 121, 151, 159, 161, 183, 186, 188, and 192 of the mature polypeptide of SEQ ID NO: 2 or SEQ ID NO: 4, wherein the variant has xylanase activity.

[2] The variant of paragraph 1, wherein the parent xylanase is (a) a polypeptide having at least 60% sequence identity to the mature polypeptide of SEQ ID NO: 2 or SEQ ID NO: 4; (b) a polypeptide encoded by a polynucleotide that hybridizes under at least low stringency conditions with the mature polypeptide coding sequence of SEQ ID NO: 1 or SEQ ID NO: 3, or the full-length complementary strand thereof; (c) a polypeptide encoded by a polynucleotide having at least 60% sequence identity to the mature polypeptide coding sequence of SEQ ID NO: 1 or SEQ ID NO: 3; or (d) a fragment of the mature polypeptide of SEQ ID NO: 2 or SEQ ID NO: 4, which has xylanase activity.

[3] The variant of paragraph 1 or 2, wherein the parent xylanase has at least 60%, e.g., at least 65%, at least 70%, at least 75%, at least 80%, at least 85%, at least 90%, %, at least 91%, at least 92%, at least 93%, at least 94%, at least 95%, at least 96%, at least 97%, at least 98%, at least 99% or 100% sequence identity to the mature polypeptide of SEQ ID NO: 2 or SEQ ID NO: 4.

[4] The variant of any of paragraphs 1-3, wherein the parent xylanase is encoded by a polynucleotide that hybridizes under low stringency conditions, medium stringency conditions, medium-high stringency conditions, high stringency conditions, or very high stringency conditions with the mature polypeptide coding sequence of SEQ ID NO: 1 or SEQ ID NO: 3, or the full-length complementary strand thereof.

[5] The variant of any of paragraphs 1-4, wherein the parent xylanase is encoded by a polynucleotide having at least 60%, e.g., at least 65%, at least 70%, at least 75%, at least 80%, at least 85%, at least 90%, %, at least 91%, at least 92%, at least 93%, at least 94%, at least 95%, at least 96%, at least 97%, at least 98%, at least 99% or 100% sequence identity to the mature polypeptide coding sequence of SEQ ID NO: 1 or SEQ ID NO: 3.

[6] The variant of any of paragraphs 1-5, wherein the parent xylanase comprises or consists of the mature polypeptide of SEQ ID NO: 2 or SEQ ID NO: 4.

[7] The variant of any of paragraphs 1-5, wherein the parent xylanase is a fragment of the mature polypeptide of SEQ ID NO: 2 or SEQ ID NO: 4, wherein the fragment has xylanase activity.

[8] The variant of any of paragraphs 1-7, which has at least 60%, e.g., at least 65%, at least 70%, at least 75%, at least 80%, at least 85%, at least 90%, at least 91%, at least 92%, at least 93%, at least 94%, at least 95% identity, at least 96%, at least 97%, at least 98%, at least 99%, but less than 100%, sequence identity to the amino acid sequence of the parent xylanase.

[9] The variant of any of paragraphs 1-8, which has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, and at least 99%, but less than 100% sequence identity to the mature polypeptide of SEQ ID NO: 2 or SEQ ID NO: 4.

[10] The variant of any of paragraphs 1-9, wherein the variant consists of 151 to 160, 161 to 170, 171 to 180, 181 to 190, 191 to 200, 201 to 210, 211 to 220, 221 to 230, 231 to 240, 241 to 250, 251 to 260, 261 to 270, or 271 to 280 amino acids.

[11] The variant of any of paragraphs 1-10, wherein the number of substitutions is 1-23, e.g., 1-15, 1-10, and 1-5, such as 1, 2, 3, 4, 5, 6, 7, 8, 9, 10, 11, 12, 13, 14, 15, 16, 17, 18, 19, 20, 21, 22, or 23 substitutions.

[12] The variant of any of paragraphs 1-11, which comprises a substitution at a position corresponding to position 2.

[13] The variant of paragraph 12, wherein the substitution is Ile.

[14] The variant of any of paragraphs 1-13, which comprises a substitution at a position corresponding to position 17.

[15] The variant of paragraph 14, wherein the substitution is Leu.

[16] The variant of any of paragraphs 1-15, which comprises a substitution at a position corresponding to position 21.

[17] The variant of paragraph 16, wherein the substitution is Ser.

[18] The variant of any of paragraphs 1-17, which comprises a substitution at a position corresponding to position 28.

[19] The variant of paragraph 18, wherein the substitution is Val.

[20] The variant of any of paragraphs 1-19, which comprises a substitution at a position corresponding to position 38.

[21] The variant of paragraph 20, wherein the substitution is Tyr or Phe.

[22] The variant of any of paragraphs 1-21, which comprises a substitution at a position corresponding to position 41.

[23] The variant of paragraph 22, wherein the substitution is Asp.

[24] The variant of any of paragraphs 1-23, which comprises a substitution at a position corresponding to position 55.

[25] The variant of paragraph 24, wherein the substitution is Asp.

[26] The variant of any of paragraphs 1-25, which comprises a substitution at a position corresponding to position 56.

[27] The variant of paragraph 26, wherein the substitution is His or Pro.

[28] The variant of any of paragraphs 1-27, which comprises a substitution at a position corresponding to position 57.

[29] The variant of paragraph 28, wherein the substitution is His.

[30] The variant of any of paragraphs 1-29, which comprises a substitution at a position corresponding to position 60.

[31] The variant of paragraph 30, wherein the substitution is Ser.

[32] The variant of any of paragraphs 1-31, which comprises a substitution at a position corresponding to position 62.

[33] The variant of paragraph 32, wherein the substitution is Thr.

[34] The variant of any of paragraphs 1-33, which comprises a substitution at a position corresponding to position 74.

[35] The variant of paragraph 34, wherein the substitution is Ala or Ser.

[36] The variant of any of paragraphs 1-35, which comprises a substitution at a position corresponding to position 81.

[37] The variant of paragraph 36, wherein the substitution is Asp.

[38] The variant of any of paragraphs 1-37, which comprises a substitution at a position corresponding to position 104.

[39] The variant of paragraph 38, wherein the substitution is Ser.

[40] The variant of any of paragraphs 1-39, which comprises a substitution at a position corresponding to position 161.

[41] The variant of paragraph 40, wherein the substitution is Leu.

[42] The variant of any of paragraphs 1-41, which comprises a substitution at a position corresponding to position 183.

[43] The variant of paragraph 42, wherein the substitution is Asp.

[44] The variant of any of paragraphs 1-43, which comprises a substitution at a position corresponding to position 186.

[45] The variant of paragraph 44, wherein the substitution is Ile or Val.

[46] The variant of any of paragraphs 1-45, which comprises a substitution at a position corresponding to position 188.

[47] The variant of paragraph 46, wherein the substitution is Ala.

[48] The variant of any of paragraphs 1-47, which comprises a substitution at a position corresponding to position 192.

[49] The variant of paragraph 48, wherein the substitution is Asp.

[50] The variant of any of paragraphs 1-49, which comprises a substitution at two positions corresponding to any of positions 2, 17, 21, 28, 38, 41, 55, 56, 57, 60, 62, 74, 81, 104, 111, 121, 151, 159, 161, 183, 186, 188, and 192.

[51] The variant of any of paragraphs 1-49, which comprises a substitution at three positions corresponding to any of positions 2, 17, 21, 28, 38, 41, 55, 56, 57, 60, 62, 74, 81, 104, 111, 121, 151, 159, 161, 183, 186, 188, and 192.

[52] The variant of any of paragraphs 1-49, which comprises a substitution at four positions corresponding to any of positions 2, 17, 21, 28, 38, 41, 55, 56, 57, 60, 62, 74, 81, 104, 111, 121, 151, 159, 161, 183, 186, 188, and 192.

[53] The variant of any of paragraphs 1-49, which comprises a substitution at five positions corresponding to any of positions 2, 17, 21, 28, 38, 41, 55, 56, 57, 60, 62, 74, 81, 104, 111, 121, 151, 159, 161, 183, 186, 188, and 192.

[54] The variant of any of paragraphs 1-49, which comprises a substitution at six positions corresponding to any of positions 2, 17, 21, 28, 38, 41, 55, 56, 57, 60, 62, 74, 81, 104, 111, 121, 151, 159, 161, 183, 186, 188, and 192.

[55] The variant of any of paragraphs 1-49, which comprises a substitution at seven positions corresponding to any of positions 2, 17, 21, 28, 38, 41, 55, 56, 57, 60, 62, 74, 81, 104, 111, 121, 151, 159, 161, 183, 186, 188, and 192.

[56] The variant of any of paragraphs 1-49, which comprises a substitution at eight positions corresponding to any of positions 2, 17, 21, 28, 38, 41, 55, 56, 57, 60, 62, 74, 81, 104, 111, 121, 151, 159, 161, 183, 186, 188, and 192.

[57] The variant of any of paragraphs 1-49, which comprises a substitution at nine positions corresponding to any of positions 2, 17, 21, 28, 38, 41, 55, 56, 57, 60, 62, 74, 81, 104, 111, 121, 151, 159, 161, 183, 186, 188, and 192.

[58] The variant of any of paragraphs 1-49, which comprises a substitution at ten positions corresponding to any of positions 2, 17, 21, 28, 38, 41, 55, 56, 57, 60, 62, 74, 81, 104, 111, 121, 151, 159, 161, 183, 186, 188, and 192.

[59] The variant of any of paragraphs 1-49, which comprises a substitution at eleven positions corresponding to any of positions 2, 17, 21, 28, 38, 41, 55, 56, 57, 60, 62, 74, 81, 104, 111, 121, 151, 159, 161, 183, 186, 188, and 192.

[60] The variant of any of paragraphs 1-49, which comprises a substitution at twelve positions corresponding to any of positions 2, 17, 21, 28, 38, 41, 55, 56, 57, 60, 62, 74, 81, 104, 111, 121, 151, 159, 161, 183, 186, 188, and 192.

[61] The variant of any of paragraphs 1-49, which comprises a substitution at thirteen positions corresponding to any of positions 2, 17, 21, 28, 38, 41, 55, 56, 57, 60, 62, 74, 81, 104, 111, 121, 151, 159, 161, 183, 186, 188, and 192.

[62] The variant of any of paragraphs 1-49, which comprises a substitution at fourteen positions corresponding to any of positions 2, 17, 21, 28, 38, 41, 55, 56, 57, 60, 62, 74, 81, 104, 111, 121, 151, 159, 161, 183, 186, 188, and 192.

[63] The variant of any of paragraphs 1-49, which comprises a substitution at fifteen positions corresponding to any of positions 2, 17, 21, 28, 38, 41, 55, 56, 57, 60, 62, 74, 81, 104, 111, 121, 151, 159, 161, 183, 186, 188, and 192.

[64] The variant of any of paragraphs 1-49, which comprises a substitution at sixteen positions corresponding to any of positions 2, 17, 21, 28, 38, 41, 55, 56, 57, 60, 62, 74, 81, 104, 111, 121, 151, 159, 161, 183, 186, 188, and 192.

[65] The variant of any of paragraphs 1-49, which comprises a substitution at seventeen positions corresponding to any of positions 2, 17, 21, 28, 38, 41, 55, 56, 57, 60, 62, 74, 81, 104, 111, 121, 151, 159, 161, 183, 186, 188, and 192.

[66] The variant of any of paragraphs 1-49, which comprises a substitution at eighteen positions corresponding to any of positions 2, 17, 21, 28, 38, 41, 55, 56, 57, 60, 62, 74, 81, 104, 111, 121, 151, 159, 161, 183, 186, 188, and 192.

[67] The variant of any of paragraphs 1-49, which comprises a substitution at nineteen positions corresponding to any of positions 2, 17, 21, 28, 38, 41, 55, 56, 57, 60, 62, 74, 81, 104, 111, 121, 151, 159, 161, 183, 186, 188, and 192.

[68] The variant of any of paragraphs 1-49, which comprises a substitution at twenty positions corresponding to any of positions 2, 17, 21, 28, 38, 41, 55, 56, 57, 60, 62, 74, 81, 104, 111, 121, 151, 159, 161, 183, 186, 188, and 192.

[69] The variant of any of paragraphs 1-49, which comprises a substitution at twenty-one positions corresponding to any of positions 2, 17, 21, 28, 38, 41, 55, 56, 57, 60, 62, 74, 81, 104, 111, 121, 151, 159, 161, 183, 186, 188, and 192.

[70] The variant of any of paragraphs 1-49, which comprises a substitution at twenty-two positions corresponding to any of positions 2, 17, 21, 28, 38, 41, 55, 56, 57, 60, 62, 74, 81, 104, 111, 121, 151, 159, 161, 183, 186, 188, and 192.

[71] The variant of any of paragraphs 1-49, which comprises a substitution at each position corresponding to positions 2, 17, 21, 28, 38, 41, 55, 56, 57, 60, 62, 74, 81, 104, 111, 121, 151, 159, 161, 183, 186, 188, and 192.

[72] The variant of any of paragraphs 1-71, which comprises one or more substitutions selected from the group consisting of V2I, F17L, A21S, E28V, S38Y,F, N41D, G55D, R56H,P, R57H, T60S, S62T, T74A,S, N81D, T104S, T111I, N121Y, N151D, H159R, M161L, N183D, L186I,V, T188A, and G192D.

[73] The variant of any of paragraphs 1-72, which comprises the substitutions V2I+R57H.

[74] The variant of any of paragraphs 1-72, which comprises the substitutions V2I+T74A.

[75] The variant of any of paragraphs 1-72, which comprises the substitutions V2I+T74S.

[76] The variant of any of paragraphs 1-72, which comprises the substitutions F17L+N81D.

[77] The variant of any of paragraphs 1-72, which comprises the substitutions F17L+M161L.

[78] The variant of any of paragraphs 1-72, which comprises the substitutions S38Y+T104S.

[79] The variant of any of paragraphs 1-72, which comprises the substitutions S38Y+L186V.

[80] The variant of any of paragraphs 1-72, which comprises the substitutions S38F+G192D.

[81] The variant of any of paragraphs 1-72, which comprises the substitutions R56P+T60S.

[82] The variant of any of paragraphs 1-72, which comprises the substitutions T74S+L186V.

[83] The variant of any of paragraphs 1-72, which comprises the substitutions T74S+L1861.

[84] The variant of any of paragraphs 1-72, which comprises the substitutions T74A+L186V.

[85] The variant of any of paragraphs 1-72, which comprises the substitutions T74A+L186I.

[86] The variant of any of paragraphs 1-72, which comprises the substitutions V2I+T74S+L186V.

[87] The variant of any of paragraphs 1-72, which comprises the substitutions F17L+N81D+T188A.

[88] The variant of any of paragraphs 1-72, which comprises the substitutions A21S+T74S+L186V.

[89] The variant of any of paragraphs 1-72, which comprises the substitutions E28V+R56H+N183D.

[90] The variant of any of paragraphs 1-72, which comprises the substitutions S38Y+T74S+L186V.

[91] The variant of any of paragraphs 1-72, which comprises the substitutions N41D+T74S+L186V.

[92] The variant of any of paragraphs 1-72, which comprises the substitutions G55D+T74S+L186V.

[93] The variant of any of paragraphs 1-72, which comprises the substitutions R57H+T74S+L186V.

[94] The variant of any of paragraphs 1-72, which comprises the substitutions S62T+T74S+L186V.

[95] The variant of any of paragraphs 1-72, which comprises the substitutions T74A+N81D+L186V.

[96] The variant of any of paragraphs 1-72, which comprises the substitutions T74S+N81D+L186V.

[97] The variant of any of paragraphs 1-72, which comprises the substitutions V2I+T74S+H159R+L186V.

[98] The variant of any of paragraphs 1-72, which comprises the substitutions V2I+F17L+T74S+L186I.

[99] The variant of any of paragraphs 1-72, which comprises the substitutions V2I+S62T+T74S+L186V.

[100] The variant of any of paragraphs 1-72, which comprises the substitutions V2I+T74S+N81D+L186V.

[101] The variant of any of paragraphs 1-72, which comprises the substitutions V2I+R57H+ T74S+L186V.

[102] The variant of any of paragraphs 1-72, which comprises the substitutions A21S+T74S+N81D+L186V.

[103] The variant of any of paragraphs 1-72, which comprises the substitutions A21S+S38Y+T74S+L186V.

[104] The variant of any of paragraphs 1-72, which comprises the substitutions A21S+G55D+T74S+L186V.

[105] The variant of any of paragraphs 1-72, which comprises the substitutions A21S+S62T+T74S+L186V.

[106] The variant of any of paragraphs 1-72, which comprises the substitutions F17L+T74S+N81D+L186V+T188A.

[107] The variant of any of paragraphs 1-72, which comprises the substitutions A21S+S38Y+T74S+N81D+L186V.

[108] The variant of any of paragraphs 1-72, which comprises the substitutions A21S+S62Y+T74S+N81D+L186V.

[109] The variant of any of paragraphs 1-72, which comprises the substitutions A21S+S38Y+S62T+T74S+L186V.

[110] The variant of any of paragraphs 1-72, which comprises the substitutions A21S+G55D+T74S+N81D+L186V.

[111] The variant of any of paragraphs 1-72, which comprises the substitutions A21S+S38Y+G55D+T74S+L186V.

[112] The variant of any of paragraphs 1-72, which comprises the substitutions A21S+G55D+S62T+T74S+L186V.

[113] The variant of any of paragraphs 1-72, which comprises the substitutions E28V+S38Y+T74S+N121Y+N151D+L186V.

[114] The variant of any of paragraphs 1-72, which comprises the substitutions A21S+S38Y+S62T+T74S+N81D+L186V.

[115] The variant of any of paragraphs 1-72, which comprises the substitutions A21S+S38Y+G55D+T74S+N81 D+L186V.

[116] The variant of any of paragraphs 1-72, which comprises the substitutions A21S+S38Y+G55D+S62T+T74S+L186V.

[117] The variant of any of paragraphs 1-72, which comprises the substitutions A21S+G55D+S62T+T74S+N81D+L186V.

[118] The variant of any of paragraphs 1-72, which comprises the substitutions V2I+E28V+S38Y+S62T+T74S+T111I+L186V.

[119] The variant of any of paragraphs 1-72, which comprises the substitutions A21S+S38Y+G55D+S62T+T74S+N81 D+L186V.

[120] The variant of any of paragraphs 1-119, which further comprises a substitution at one or more positions corresponding to positions 19, 23, 84, and 88.

[121] The variant of paragraph 120, wherein the number of further substitutions is 1-4, such as 1, 2, 3, or 4 substitutions.

[122] The variant of paragraph 120 or 121, which comprises a substitution at a position corresponding to position 19.

[123] The variant of paragraph 122, wherein the substitution is with Ala.

[124] The variant of any of paragraphs 120-123, which comprises a substitution at a position corresponding to position 23.

[125] The variant of paragraph 124, wherein the substitution is with Pro.

[126] The variant of any of paragraphs 120-125, which comprises a substitution at a position corresponding to position 84.

[127] The variant of paragraph 126, wherein the substitution is with Pro.

[128] The variant of any of paragraphs 120-127, which comprises a substitution at a position corresponding to position 88.

[129] The variant of paragraph 128, wherein the substitution is with Thr.

[130] The variant of any of paragraphs 120-129, which comprises a substitution at two positions corresponding to any of positions 19, 23, 84, and 88.

[131] The variant of any of paragraphs 120-129, which comprises a substitution at three positions corresponding to any of positions 19, 23, 84, and 88.

[132] The variant of any of paragraphs 120-129, which comprises a substitution at each position corresponding to positions 19, 23, 84, and 88.

[133] The variant of any of paragraphs 120-132, which comprises one or more substitutions selected from the group consisting of T19A, G23P, V84P, and I88T.

[134] The variant of any of paragraphs 120-133, which comprises the substitutions T19A+G23P.

[135] The variant of any of paragraphs 120-133, which comprises the substitutions T19A+V84P.

[136] The variant of any of paragraphs 120-133, which comprises the substitutions T19A+I88T.

[137] The variant of any of paragraphs 120-133, which comprises the substitutions G23P+V84P.

[138] The variant of any of paragraphs 120-133, which comprises the substitutions G23P+I88T.

[139] The variant of any of paragraphs 120-133, which comprises the substitutions V84P+I88T.

[140] The variant of any of paragraphs 120-133, which comprises the substitutions T19A+G23P+V84P.

[141] The variant of any of paragraphs 120-133, which comprises the substitutions T19A+G23P+I88T.

[142] The variant of any of paragraphs 120-133, which comprises the substitutions G23P+V84P+I88T.

[143] The variant of any of paragraphs 120-133, which comprises the substitutions T19A+V84P+I88T.

[144] The variant of any of paragraphs 120-133, which comprises the substitutions T19A+G23P+V84P+I88T.

[145] An isolated polynucleotide encoding the variant of any of paragraphs 1-144.

[146] A nucleic acid construct comprising the polynucleotide of paragraph 145.

[147] An expression vector comprising the polynucleotide of paragraph 145.

[148] A host cell comprising the polynucleotide of paragraph 145.

[149] A method of producing a variant having xylanase activity, comprising: (a) cultivating a host cell comprising the polynucleotide of paragraph 145 under conditions suitable for the expression of the variant; and (b) recovering the variant.

[150] A transgenic plant, plant part or plant cell transformed with the polynucleotide of paragraph 145.

[151] A method of producing a variant of any of paragraphs 1-144, comprising: cultivating a transgenic plant or a plant cell comprising a polynucleotide encoding the variant under conditions conducive for production of the variant; and recovering the variant.

[152] A method for obtaining the variant of any of paragraphs 1-144, comprising introducing into the parent xylanase a substitution at one or more positions corresponding to positions 2, 17, 21, 28, 38, 41, 55, 56, 57, 60, 62, 74, 81, 104, 111, 121, 151, 159, 161, 183, 186, 188, and 192 of the mature polypeptide of SEQ ID NO: 2 or SEQ ID NO: 4, wherein the variant has xylanase activity; and recovering the variant.

[153] A method of degrading a xylan-containing material by treating the material with a variant of any of paragraphs 1-144.

[154] A method for treating a pulp, comprising contacting the pulp with a variant of any of paragraphs 1-144.

[155] The method of paragraph 154, wherein the treating of the pulp with the variant increases the brightness of the pulp at least 1.05-fold, e.g., at least 1.1-fold, at least 1.2-fold, at least 1.3-fold, at least 1.4-fold, at least 1.5-fold, at least 2-fold, at least 3-fold, at least 4-fold, at least 5-fold, or at least 10-fold compared to treatment with the parent.

[156] A method for producing xylose, comprising contacting a xylan-containing material with a variant of any of paragraphs 1-144.

[157] The method of paragraph 156, further comprising recovering the xylose.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

Various references are cited herein, the disclosures of which are incorporated by reference in their entireties.

1. An isolated variant of a parent xylanase, comprising a substitution at one or more positions corresponding to positions 2, 17, 21, 28, 38, 41, 55, 56, 57, 60, 62, 74, 81, 104, 111, 121, 151, 159, 161, 183, 186, 188, and 192 of the mature polypeptide of SEQ ID NO: 2 or SEQ ID NO: 4, wherein the variant has xylanase activity.

2. The variant of claim 1, wherein the parent xylanase is
(a) a polypeptide having at least 60% sequence identity to the mature polypeptide of SEQ ID NO: 2 or SEQ ID NO: 4;
(b) a polypeptide encoded by a polynucleotide that hybridizes under at least low stringency conditions with the mature polypeptide coding sequence of SEQ ID NO: 1 or SEQ ID NO: 3, or the full-length complementary strand thereof;
(c) a polypeptide encoded by a polynucleotide having at least 60% sequence identity to the mature polypeptide coding sequence of SEQ ID NO: 1 or SEQ ID NO: 3; or
(d) a fragment of the mature polypeptide of SEQ ID NO: 2 or SEQ ID NO: 4, which has xylanase activity.

3. The variant of claim 1, wherein the parent xylanase comprises or consists of the mature polypeptide of SEQ ID NO: 2 or SEQ ID NO: 4, or a fragment thereof having xylanase activity.

4. The variant of claim 1, which has at least 60%, e.g., at least 65%, at least 70%, at least 75%, at least 80%, at least 85%, at least 90%, at least 91%, at least 92%, at least 93%, at least 94%, at least 95% identity, at least 96%, at least 97%, at least 98%, at least 99%, but less than 100%, sequence identity to the amino acid sequence of the parent xylanase.

5. The variant of claim 1, wherein the number of substitutions is 1-23, e.g., 1-15, 1-10, and 1-5, such as 1, 2, 3, 4, 5, 6, 7, 8, 9, 10, 11, 12, 13, 14, 15, 16, 17, 18, 19, 20, 21, 22, or 23 substitutions.

6. The variant of claim 1, which comprises one or more substitutions selected from the group consisting of V2I, F17L, A21S, E28V, S38Y,F, N41D, G55D, R56H,P, R57H, T60S, S62T, T74A,S, N81D, T104S, T111I, N121Y, N151D, H159R, M161L, N183D, L186I,V, T188A, and G192D.

7. The variant of claim 1, which further comprises a substitution at one or more positions corresponding to positions 19, 23, 84, and 88.

8. The variant of claim 7, wherein the number of further substitutions is 1-4, such as 1, 2, 3, or 4 substitutions.

9. The variant of claim 7, which comprises one or more substitutions selected from the group consisting of T19A, G23P, V84P, and I88T.

10. An isolated polynucleotide encoding the variant of claim 1.

11. A host cell comprising the polynucleotide of claim 10.

12. A method of producing a variant having xylanase activity, comprising:
(a) cultivating a host cell comprising the polynucleotide of claim 10 under conditions suitable for the expression of the variant; and
(b) recovering the variant.

13. A transgenic plant, plant part or plant cell transformed with the polynucleotide of claim 10.

14. A method of producing the variant of claim 1, comprising:
(a) cultivating a transgenic plant or a plant cell comprising a polynucleotide encoding the variant under conditions conducive for production of the variant; and
(b) recovering the variant.

15. A method for obtaining the variant of claim 1, comprising introducing into the parent xylanase a substitution at one or more positions corresponding to positions 2, 17, 21, 28, 38, 41, 55, 56, 57, 60, 62, 74, 81, 104, 111, 121, 151, 159, 161, 183, 186, 188, and 192 of the mature polypeptide of SEQ ID NO: 2 or SEQ ID NO: 4, wherein the variant has xylanase activity; and recovering the variant.

16. A method of degrading a xylan-containing material by treating the material with the variant of claim 1.

17. A method for treating a pulp, comprising contacting the pulp with the variant of claim 1.

18. The method of claim 17, wherein the treating of the pulp with the variant increases the brightness of the pulp at least 1.05-fold, e.g., at least 1.1-fold, at least 1.2-fold, at least 1.3-fold, at least 1.4-fold, at least 1.5-fold, at least 2-fold, at least 3-fold, at least 4-fold, at least 5-fold, or at least 10-fold compared to treatment with the parent.

19. A method for producing xylose, comprising contacting a xylan-containing material with the variant of claim 1.

20. The method of claim 19, further comprising recovering the xylose.


SEQUENCE LISTING (64 sequences)

SEQ ID NO: 1 - DNA, 666 bp, Thermobifida fusca
  CDS (1)..(663)
  sig_peptide (1)..(81)
  mat_peptide (82)..(663)
atgaagaaacctcttggcaaaatcgttgcgtcaactgctcttcttatctctgtagctttctcatcttcaatcgcttctgctgcagtaacttctaacgagacaggttaccatgacggctatttctattcattttggacagacgctcctggaactgtatcaatggagcttggacctggtggcaactactcaacgtcttggcgcaacacaggaaacttcgtagcaggtaaaggctgggcgacaggaggtcgtcgcacagttacttactcagcttctttcaacccttctggtaacgcttatcttacactttacggatggactcgcaaccctcttgttgagtactatatcgttgagtcatggggcacttatcgccctacaggcacttacatgggtactgtaactactgatggtggcacttacgacatctacaaaactacacgctacaacgctcctagcatcgagggcactcgcacattcgaccaatactggtctgtacgccagtcaaaacgcacatctggcactatcactgctggcaaccattttgacgcatgggctcgccacggaatgcatcttggaacgcacgactacatgatcatggctacagagggttaccaaagctcaggctcatcaaacgttacacttggcacatcaggaggtggcaacccttaa

SEQ ID NO: 2 - protein, 221 aa, Thermobifida fusca
MKKPLGKIVASTALLISVAFSSSIASAAVTSNETGYHDGYFYSFWTDAPGTVSMELGPGGNYSTSWRNTGNFVAGKGWATGGRRTVTYSASFNPSGNAYLTLYGWTRNPLVEYYIVESWGTYRPTGTYMGTVTTDGGTYDIYKTTRYNAPSIEGTRTFDQYWSVRQSKRTSGTITAGNHFDAWARHGMHLGTHDYMIMATEGYQSSGSSNVTLGTSGGGNP

SEQ ID NO: 3 - DNA, 1017 bp, Thermobifida fusca
  sig_peptide (1)..(126)
  CDS (1)..(1014)
  mat_peptide (127)..(1014)
atgaaccatgcccccgccagtctgaagagccggagacgcttccggcccagactgctcatcggcaaggcgttcgccgcggcactcgtcgcggtcgtcacgatgatccccagtactgccgcccacgcggccgtgacctccaacgagaccgggtaccacgacgggtacttctactcgttctggaccgacgcgcctggaacggtctccatggagctgggccctggcggaaactacagcacctcctggcggaacaccgggaacttcgtcgccggtaagggatgggccaccggtggccgccggaccgtgacctactccgccagcttcaacccgtcgggtaacgcctacctgaccctctacgggtggacgcggaacccgctcgtggagtactacatcgtcgaaagctggggcacctaccggcccaccggtacctacatgggcacggtgaccaccgacggtggtacctacgacatctacaagaccacgcggtacaacgcgccctccatcgaaggcacccggaccttcgaccagtactggagcgtccgccagtccaagcggaccagcggtaccatcaccgcggggaaccacttcgacgcgtgggcccgccacggtatgcacctcggaacccacgactacatgatcatggcgaccgagggctaccagagcagcggatcctccaacgtgacgttgggcaccagcggcggtggaaaccccggtgggggcaacccccccggtggcggcaacccccccggtggcggtggctgcacggcgacgctgtccgcgggccagcagtggaacgaccgctacaacctcaacgtcaacgtcagcggctccaacaactggaccgtgaccgtgaacgttccgtggccggcgaggatcatcgccacctggaacatccacgccagctacccggactcccagaccttggttgcccggcctaacggcaacggcaacaactggggcatgacgatcatgcacaacggcaactggacgtggcccacggtgtcctgcagcgccaactag

SEQ ID NO: 4 - protein, 338 aa, Thermobifida fusca
MNHAPASLKSRRRFRPRLLIGKAFAAALVAVVTMIPSTAAHAAVTSNETGYHDGYFYSFWTDAPGTVSMELGPGGNYSTSWRNTGNFVAGKGWATGGRRTVTYSASFNPSGNAYLTLYGWTRNPLVEYYIVESWGTYRPTGTYMGTVTTDGGTYDIYKTTRYNAPSIEGTRTFDQYWSVRQSKRTSGTITAGNHFDAWARHGMHLGTHDYMIMATEGYQSSGSSNVTLGTSGGGNPGGGNPPGGGNPPGGGGCTATLSAGQQWNDRYNLNVNVSGSNNWTVTVNVPWPARIIATWNIHASYPDSQTLVARPNGNGNNWGMTIMHNGNWTWPTVSCSAN

SEQ ID NO: 5 - DNA, 48 bp, Thermobifida fusca
atcagtttgaaaattatgtattatggagctctataaaaatgaggaggg

SEQ ID NO: 6 - DNA, 40 bp, Thermobifida fusca
ctttaaccgcacagcgtttttttattgattaacgcgttta

SEQ ID NO: 7 - DNA, 33 bp, Thermobifida fusca
catcaaacgttacagtaggcacatcaggaggtg

SEQ ID NO: 8 - DNA, 33 bp, Thermobifida fusca
cacctcctgatgtgcctactgtaacgtttgatg

SEQ ID NO: 9 - DNA, 32 bp, Thermobifida fusca
ggtaacgcttatcttgcactttacggatggac

SEQ ID NO: 10 - DNA, 32 bp, Thermobifida fusca
gtccatccgtaaagtgcaagataagcgttacc

SEQ ID NO: 11 - DNA, 33 bp, Thermobifida fusca; identical to SEQ ID NO: 7

SEQ ID NO: 12 - DNA, 32 bp, Thermobifida fusca
ggtaacgcttatctttcactttacggatggac

SEQ ID NO: 13 - DNA, 32 bp, Thermobifida fusca; identical to SEQ ID NO: 12

SEQ ID NO: 14 - DNA, 32 bp, Thermobifida fusca
gtccatccgtaaagtgaaagataagcgttacc

SEQ ID NO: 15 - DNA, 32 bp, Thermobifida fusca; identical to SEQ ID NO: 9

SEQ ID NO: 16 - DNA, 32 bp, Thermobifida fusca; identical to SEQ ID NO: 10

SEQ ID NO: 17 - DNA, 33 bp, Thermobifida fusca; identical to SEQ ID NO: 7

SEQ ID NO: 18 - DNA, 32 bp, Thermobifida fusca; identical to SEQ ID NO: 9

SEQ ID NO: 19 - DNA, 33 bp, Thermobifida fusca; identical to SEQ ID NO: 7

SEQ ID NO: 20 - DNA, 32 bp, Thermobifida fusca; identical to SEQ ID NO: 12

SEQ ID NO: 21 - DNA, 30 bp, Thermobifida fusca
attttggacagactctcctggaactgtatc

SEQ ID NO: 22 - DNA, 33 bp, Thermobifida fusca; identical to SEQ ID NO: 7

SEQ ID NO: 23 - DNA, 32 bp, Thermobifida fusca; identical to SEQ ID NO: 12

SEQ ID NO: 24 - DNA, 31 bp, Thermobifida fusca
gcaactactcaacgtactggcgcaacacagg

SEQ ID NO: 25 - DNA, 33 bp, Thermobifida fusca; identical to SEQ ID NO: 7

SEQ ID NO: 26 - DNA, 32 bp, Thermobifida fusca; identical to SEQ ID NO: 12

SEQ ID NO: 27 - DNA, 32 bp, Thermobifida fusca
ggctgggcgacaggagaccgtcgcacagttac

SEQ ID NO: 28 - DNA, 33 bp, Thermobifida fusca; identical to SEQ ID NO: 7

SEQ ID NO: 29 - DNA, 32 bp, Thermobifida fusca; identical to SEQ ID NO: 12

SEQ ID NO: 30 - DNA, 33 bp, Thermobifida fusca
tacggatggactcgcgaccctcttgttgagtac

SEQ ID NO: 31 - DNA, 33 bp, Thermobifida fusca; identical to SEQ ID NO: 7

SEQ ID NO: 32 - DNA, 32 bp, Thermobifida fusca; identical to SEQ ID NO: 12

SEQ ID NO: 33 - DNA, 35 bp, Thermobifida fusca
cgcacagttacttacactgcttctttcaacccttc

SEQ ID NO: 34 - DNA, 20 bp, Thermobifida fusca
gccttactatacctaacatg

SEQ ID NO: 35 - DNA, 25 bp, Thermobifida fusca
gaatttaggaggcttacttgtctgc

SEQ ID NO: 36 - DNA, 32 bp, Thermobifida fusca; identical to SEQ ID NO: 12

SEQ ID NO: 37 - DNA, 32 bp, Thermobifida fusca; identical to SEQ ID NO: 14

SEQ ID NO: 38 - DNA, 32 bp, Thermobifida fusca; identical to SEQ ID NO: 9

SEQ ID NO: 39 - DNA, 32 bp, Thermobifida fusca; identical to SEQ ID NO: 10

SEQ ID NO: 40 - DNA, 33 bp, Thermobifida fusca; identical to SEQ ID NO: 7

SEQ ID NO: 41 - DNA, 32 bp, Thermobifida fusca; identical to SEQ ID NO: 12

SEQ ID NO: 42 - DNA, 32 bp, Thermobifida fusca; identical to SEQ ID NO: 27

SEQ ID NO: 43 - DNA, 32 bp, Thermobifida fusca
cgcttctgctgcaatcacttctaacgagacag

SEQ ID NO: 44 - DNA, 32 bp, Thermobifida fusca
gcgacaggaggtcgtcatacagttacttactc

SEQ ID NO: 45 - DNA, 32 bp, Thermobifida fusca; identical to SEQ ID NO: 44

SEQ ID NO: 46 - DNA, 32 bp, Thermobifida fusca
gagtaagtaactgtatgacgacctcctgtcgc

SEQ ID NO: 47 - DNA, 32 bp, Thermobifida fusca
ctcaacgtcttggcgcgacacaggaaacttcg

SEQ ID NO: 48 - DNA, 32 bp, Thermobifida fusca
cgaagtttcctgtgtcgcgccaagacgttgag

SEQ ID NO: 49 - DNA, 33 bp, Thermobifida fusca; identical to SEQ ID NO: 30

SEQ ID NO: 50 - DNA, 31 bp, Thermobifida fusca; identical to SEQ ID NO: 24

SEQ ID NO: 51 - DNA, 35 bp, Thermobifida fusca; identical to SEQ ID NO: 33

SEQ ID NO: 52 - DNA, 32 bp, Thermobifida fusca; identical to SEQ ID NO: 27

SEQ ID NO: 53 - DNA, 32 bp, Thermobifida fusca; identical to SEQ ID NO: 27

SEQ ID NO: 54 - DNA, 32 bp, Thermobifida fusca
gtaactgtgcgacggtctcctgtcgcccagcc

SEQ ID NO: 55 - DNA, 32 bp, Thermobifida fusca; identical to SEQ ID NO: 27

SEQ ID NO: 56 - DNA, 32 bp, Thermobifida fusca; identical to SEQ ID NO: 54

SEQ ID NO: 57 - DNA, 35 bp, Thermobifida fusca; identical to SEQ ID NO: 33

SEQ ID NO: 58 - DNA, 35 bp, Thermobifida fusca
gaagggttgaaagaagcagtgtaagtaactgtgcg

SEQ ID NO: 59 - DNA, 32 bp, Thermobifida fusca; identical to SEQ ID NO: 27

SEQ ID NO: 60 - DNA, 32 bp, Thermobifida fusca; identical to SEQ ID NO: 54

SEQ ID NO: 61 - DNA, 32 bp, Thermobifida fusca; identical to SEQ ID NO: 27

SEQ ID NO: 62 - DNA, 32 bp, Thermobifida fusca; identical to SEQ ID NO: 54

SEQ ID NO: 63 - DNA, 35 bp, Thermobifida fusca; identical to SEQ ID NO: 33

SEQ ID NO: 64 - DNA, 35 bp, Thermobifida fusca; identical to SEQ ID NO: 58